(19) United States
(12) Patent Application Publication
(10) Pub. No.: US 2026/0234548 A1
(43) Pub. Date: Aug. 13, 2026

(54) Tailoring the Secretion of Biological Factors

(71) Applicant: Ronawk, Inc., Overland Park, KS (US)

(72) Inventors: Adam J. Mellott, Parkville, MO (US); Jacob G. Hodge, Merriam, KS (US); Heather Decker, Kansas City, KS (US); Jennifer L. Robinson, Lake Forest Park, WA (US); David S. Zamierowski, Overland Park, KS (US)

(21) Appl. No.: 19/152,589

(22) PCT Filed: Mar. 4, 2024
(86) PCT No.: PCT/US2024/018298

§ 371 Date: Jul. 31, 2025

Related U.S. Application Data
(60) Provisional application No. 63/449,971, filed on Mar. 4, 2023.

(51) Int. Cl. C12N 5/00; C12N 5/0775

(52) U.S. Cl. CPC C12N 5/0062; C12N 5/0667; C12N 2503/02; C12N 2513/00; C12N 2533/30; C12N 2539/00

(57) ABSTRACT

Methods of externally reprogramming how cells behave and which biological factors are secreted from cells, such as by introducing an external (controllable) stimuli for obtaining a desired outcome for producing a target biological factor, are provided.

### CROSS-REFERENCE TO RELATED APPLICATIONS

This application claims priority to U.S. Provisional Application No. 63/449,971, filed Mar. 4, 2023, which is expressly incorporated by reference herein in its entirety.

### TECHNICAL FIELD

Embodiments of the presently-disclosed invention relate generally to methods of externally reprogramming how cells behave and which biological factors are secreted from cells, such as by introducing an external (controllable) stimuli for obtaining a desired outcome for producing a target biological factor.

### BACKGROUND

When it comes to maintaining cell phenotypes and functions, cells must constantly communicate with each other to maintain homeostasis in dynamic environments. One of the ways in which cells communicate with each other is through the secretion of vesicles (e.g., extracellular vesicles). Lipid bilayer vesicles that range in diameter from 30-100 nm are known as exosomes and traditionally transport proteins such as enzymes, oligonucleotides such as DNA, mRNA, ncRNA, microRNA lipids, and other contents or biomarkers. Vesicles that range in diameter from 50-1,000 nm are known as microvesicles and can carry greater loads of cargo than exosomes. Vesicles that range in diameter from 500-2,000 nm are known as apoptotic bodies and can carry larger cargoes such a organelles in addition to proteins, enzymes, growth factors, cytokines, oligonucleotides, lipids and other cargoes. These vesicles are often secreted by one cell and taken up by another cell. Exosomes are one of the most common vesicles secreted by cells, and are present in biofluids such as serum, plasma, urine, seminal fluid, cerebrospinal fluid (CSF), saliva, tears, and breast milk. Exosomes are stable in these fluids and can pass through the blood brain barrier. Hence, exosomes are being researched as therapeutic agents to transport different drugs and are being researched as biomarkers for different diseases, cancers, aging, injuries, and as prognostic indicators in response to treatments.

Exosomes can be taken up by adjacent cells or travel through the body to be taken up by cells that are far away from the cell or origin where the exosome was secreted. Exosomes can be secreted by epithelial cells, endothelial cells, mesenchymal stem cells (MSCs), macrophages, dendritic cells, tumor cells, neurons, oligodendrocytes, reticulocytes, mast cells, platelets, cancer cells, B cells, T cells, and astrocytes. Several researchers are exploring and developing methods to stimulate the secretion of exosomes from target cells and isolate exosomes for therapy and diagnostic development. Most production methods rely on exposing target cells to specific media formulations that are depleted of serum and other protein content to “starve” the cells temporarily. Furthermore, cells are grown in two-dimensional (2D) on culture plastics, and in bioreactors on microcarriers. However, the ability to scale the production of exosomes, let alone the quality, and specific type of exosome carrying target cargoes is extremely challenging.

Therefore, there remains a need in the art for methods and/or platforms that may increase production of exosomes in MSCs while enabling the tailoring of exosomes secreted by MSCs in culture, such as by introducing controllable stimuli to the MSCs to produce a desired outcome.

### SUMMARY OF INVENTION

One or more embodiments of the invention may address one or more of the aforementioned problems. Certain embodiments according to the invention provide methods of characterizing a therapeutically desirable constituent or constituent profile of a secretome composition secreted from a cell type of interest. The methods may comprise the following: (i) seeding a first tissue mimetic three-dimensional (3D) hydrogel block (TMHB) comprising a macrostructure defined by a continuous polymeric matrix material and a network of microporous channels and/or chambers extending throughout the continuous polymeric matrix material with a first cell type of interest; (ii) simulating in vitro cell growth and propagation of the first cell type of interest by feeding the first cell type of interest with a first culture media and allowing the first cell type of interest to propagate throughout the first TMHB and to secrete the secretome composition; (iv) collecting a secretome composition; (v) subjecting the secretome composition to a stratification operation based on molecular weight cut-off values providing a plurality of different strata each characterized by a different range of constituent-molecular weights; (vi) characterizing and/or identifying one or more individual constituents present with each strata of interest; and (v) testing each strata of interest for relative therapeutic benefit either by in vivo analysis or ex vivo analysis and identifying the strata of interest that provides a highest therapeutic benefit as the therapeutically constituent profile.

In another aspect, the present invention provides a method of tailoring a constituent profile of a natural secretome composition secreted from a cell type of interest, comprising: (i) seeding a first tissue mimetic three-dimensional (3D) hydrogel block (TMHB) comprising a macrostructure defined by a continuous polymeric matrix material and a network of microporous channels and/or chambers extending throughout the continuous polymeric matrix material with a first cell type of interest; (ii) feeding first cell type of interest with a first culture media;
(iii) introducing at least one external stimuli configured to modify the natural secretome composition secreted from a cell type of interest to more closely track a target secretome composition (e.g., a previously identified therapeutically beneficial constituent or profile thereof) having a target constituent or target constituent profile; wherein the at least one external stimuli comprises one or more controllable parameters, such as addition of a coating on an interface between the continuous polymeric matrix material and the network of microporous channels and/or chambers, adjusting an oxygen concentration, a pH, a temperature, electromagnetic properties, and fluid dynamics through the first TMHB; (iv) allowing the first cell type of interest to propagate throughout the first TMHB and to secrete the secretome composition; and (iv) collecting a modified secretome composition that is different than the natural secretome composition due to the external stimuli.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates two (2) separate TMHBs of the IPHB variety in accordance with certain embodiments of the invention;

FIG. 2 illustrates three (3) interlocked IPHBs in accordance with certain embodiments of the invention;

FIG. 3 depicts top and side views of an IPHB and illustrates the continuous polymeric matrix material and the network of microporous channels and/or chambers extending throughout the continuous polymeric matrix material, which are exposed at the surfaces of the IPHB, in accordance with certain embodiments of the invention;

FIG. 4 illustrates a IPHB including an interlocking-male component located on a top surface and an interlocking-female component protruding into the bottom surface in accordance with certain embodiments of the invention;

FIG. 5 illustrates another IPHB including an interlocking-male component located on a top surface and an interlocking-female component protruding into the bottom surface in accordance with certain embodiments of the invention;

FIG. 6 illustrates cell migration from a first IPHB to a second IPHB when interlocked together in accordance with certain embodiments of the invention;

FIGS. 7A-H relate generally to Characterization of ASC populations. Characterization of initial ASC P1 population with A Spindle/Mesenchymal-like adherent cells with trilineage/multipotent potential, B CD surface marker expression of CD73/90/105/271 [Scale bar=100 lm; negative markers of CD34 and CD45 exhibited less than 5% positive—data not shown], and C Gene expression of key markers via MSC Phenotyping array. D Photograph of the 3D-printed, −1 cm³ tissue-mimetic X-Block within 6-well culture vessel. The X-Blocks are printed with a predefined microarchitecture that results in a substantial increase in surface area-to-volume via the formation of macrostructure. Additionally, the X-Blocks were fabricated to mimic the mechanical and viscoelastic properties native adipose tissue. E In situ cell number quantification within 3D system (X-Block). ASCs were then extracted from 2D or 3D and re-plated in 2D for analysis after 1-week (P2 for 2D) and 2-week (P3 for 2D) to assess for F Population Doubling Time, G Metabolic activity, and H Mitochon-drial activity (membrane potential). Black dashed line indicates initial P1 population. Error bars are s.e.m. Significance denoted as *p<0.05, **p<0.01, ***p<0.001, and ****p<0.0001, n=4;

FIG. 8 relates to ASC Growth Curve for 2D and 3D to Establish ASC-CM Collection Days. ASCs cultured in the 3D system had a slight delay in reaching the growth phase likely due to an initial migratory phase to equally distribute throughout hydrogel microarchitecture, but quickly overcame the relative cell number in 2D and eventually exhibited a higher rate of proliferation in 3D. Therefore, cell numbers were assessed in advance for 2D and 3D to determine the optimal days for collection of ASC-CM to standardize relative media-per-cell ratios. Based on prior literature, collection of ASC-CM in 2D at 6080% confluency was desired and days 6-8 were selected to collect ASC-CM. Media volumes were adjusted accordingly to account for differences in cell numbers between 2D and 3D;

FIG. 9A-I illustrates larger MW secretome fraction is key driver of KC wound healing activity. A Schematic diagram of ASC-CM centrifugation filtration steps for each molecular weight (MW) kDa cutoff. Upper chamber solution considered concentrate for that range; residual lower chamber filtrate used for next MW filtration step. B ASC-CM protein content for each MW fraction. C Representative morphology images of KCs after treatment with KC-GM supplemented with different ASC-CM concentrates [Scale bar=100 lm]. D Representative image panel of KCs treated with ASC-CM fractions from 2D or 3D and stained with Hoechst (Blue) and Vimentin (Cyan) [Scale bar=50 lm]. E Image quantification of Vimentin immunolabeling of KCs (relative to baseline control KCs). F qRT-PCR analysis of p16ink4a (senescence marker) of KCs after treatment with fractionated ASC-CM for 24 h. Dashed line is baseline control KCs. G Relative change in metabolic activity (via PrestoBlue). H Relative change in proliferation (via PicoGreen). I Relative change in migration (via scratch assay). Significance denoted as *p<0.05, **p<0.01, ***p<0.001, and ****p<0.0001, n=4;

FIG. 10 shows Image Analysis of KC Senescence after MW Stratified ASC-CM Treatment. Representative images of KCs after treatment with KC-GM supplemented with different ASC-CM MW concentrates for 24 hours. B-Galactosidase activity was used as a surrogate measure for senescence. Lower MW fractions of ASC-CM appeared to induce senescence in KC populations. This corroborated the qRT-PCR data for p16 previously discussed;

FIGS. 11A-D illustrates ASC populations within tissue-mimetic system favor secretion of EVs. A ASC-CM was processed for EV collection/isolation/purification from 2D and 3D culture and evaluated via protein content (QuickDrop/BCA/Bradford) of the EV fraction (left) and particle counts with NTA (right) to quantify EV concentrations. B Relative composition of EVs within ASC-CM was then quantified relative to protein content of “100 kDa” fraction (left) and total (i.e., “Full”) secreted protein content (right). C Analysis of EV size distribution for 2D and 3D was assessed with NTA data, and D Cumulative frequency distribution was generated to determine what percentage of measured particles fell within the standard exosome range (25-250 nm). Teal patterned bars indicate “within exosome size” range, whereas black bars are too large to likely be exosomes. Significance denoted as *p<0.05, **p<0.01, ***p<0.001, and ****p<0.0001, n=4;

FIGS. 12 A-E illustrates ASC-EVs within tissue-mimetic system contain more potent re-epithelialization stimulus. A Schematic diagram of ASC-CM filtration via 100-kDa centrifuge filter followed by subsequent EV/exosome isolation from the “100 kDa” concentrate in upper chamber. Isolated 2D-EVs (yellow circles) and 3D-EVs (blue circles) were then reapplied back to the “<100 kDa Filtrate” samples. The effect of ASC-CM “<100 kDa Filtrate” from 2D (silhouette) and 3D (patterned), with/without EVs, was evaluated for ability to modulate KC B metabolic, C proliferative, and D migratory activity. E Evaluation of KC morphological changes after treatment of “<100 kDa Filtrate” without EVs (top row) and with EVs (bottom row) [Scale bar=100 lm]. Significance denoted as *p<0.05, **p<0.01, ***p<0.001, and ****p<0.0001, n=4;

FIGS. 13 A-B illustrate 3D-EVs enhance expression of basal and suprabasal cytokeratins in a dose-dependent manner. A Representative images of KCs stained for cytokeratins (Red), including Keratin 5 (top row), Keratin 10 (middle row), and Keratin 16 (bottom row). KCs treated with KC-GM served as a control for baseline expression (left column), and 2D-EVs (middle column) and 3D-EVs (right column) at the highest dose of 250 lg/mL were the treatment groups [Scale bar=100 lm, Inset Scale bar=20 lm]. B ASC-EVs treatment of KCs at different doses from 2D (silhouette) and 3D (patterned) were evaluated via qRT-PCR analysis of K5, K10, and K16. GAPDH was used as an internal control. Values are represented as relative fold change to baseline control KCs indicated by dashed line. Significance denoted as *p<0.05, **p<0.01, ***p<0.001, and ****p<0.0001, n=3;

FIGS. 14 A-E illustrates EMT and epidermal regeneration of KCs exhibit dose-dependent response to ASC-EVs. A The effect of ASC-EVs at different doses from 2D (silhouette) and 3D (patterned) was evaluated for ability to modulate KC metabolic (top row), proliferative (middle row), and migratory activity (bottom row). B Representative images of KC morphology and cytoskeletal changes after ASC-EV treatment. Inset is a brightfield image. Stains include Hoechst (Blue), Phalloidin (Green), DiI-labeled exosomes (Red). White arrow denotes formation of actin cap (green phalloidin). [Scale bar=100 lm]. C qRT-PCR analysis of CCND1, VIM, FLG, TWIST1, CDH1, and CDH2. GAPDH was used as an internal control. Values are represented as relative fold change to baseline control KCs indicated by dashed line. D The relative ratio for RNA expression of N-Cadherin (CDH2) to E-Cadherin (CDH1). E Tabulated values for all genes evaluated by qRT-PCR and their respective R² values and relative directionality of correlation. Significance denoted as *p<0.05, **p<0.01, ***p<0.001, and ****p<0.0001, n=3;

FIG. 15 shows comparative KC morphological changes with/without dilabeled EVs. ASC-EVs were isolated from both 2D and 3D systems, labeled with a lipophilic dye, DiI (red), and dosed into KC-GM at 250 j·tg/mL for treatment of KCs and assessment of morphological changes. KC morphology under transmitted light (top row), low magnification (20×) of fluorescent images of KCs with phalloidin (green) and Hoechst (blue) to depict formation of cell sheets in 3D-EV treated group, and high magnification (60×) of DiI-labeled EVs (red) to assess perinuclear localization of EVs. Control KC group (left column) compared to 2D-EV treated (middle column) and 3D-EV treated (right column). Scale bar for 20× images=100 μm, for 60× images=25 μm;

FIG. 16 shows KC morphological changes after MW stratified ASC-CM treatment. Representative morphology images of KCs after treatment with KC-GM supplemented with different ASC-CM MW concentrates for 24 hours. Only the “Full” and “>100 kDa” fractions induce similar KC morphological changes, including spindle-cell formation, stratification, and collective cell sheet formation. KC morphology changes were seen to varying extents for both 2D and 3D ASC-CM. Moreover, CellProfiler was used to assess nuclear area and circularity. KCs treated with “Full” or “<100 kDa” fraction exhibited nuclei with a larger surface area and less circular shape (0-1 scale with 0.5=to a circle). Likely a result, in part, to cell flattening and consequently, nuclear flattening;

FIGS. 17 A-D illustrates the characterization of ASC phenotype and substrate coating. Characterization of the initial ASC P1 population with (A) Spindle/Mesenchymal-like adherent cells with trilineage/multipotent potential and (B) positive CD surface marker expression of CD73/90/105/271 and negative expression of CD34/45 [Scale bar=100 μm]. (C) Photographs of ~1 cm³ tissue-mimetic X-Block inserted within a 6-well culture vessel. The textured appearance of the hydrogel is a result of the microporous architecture that traverses throughout the entire hydrogel. (D) ASCs were cultured on collagen type I, fibronectin, and fibrin coating 2D and 3D surface to assess for phenotypic and morphological changes;

FIG. 18 shows fluorescent imaging of ASCs cultured within the 3D hydrogel system. ASCs were seeded and cultured within the 3D Hydrogel system, fixed with 4% PFA, and labeled with Hoechst (Blue), Phalloidin (Green), or MitoTracker (Magenta). Low magnification (4×) of the labeled ASCs within the hydrogel system (Far Left Panel) is paired with higher magnification (20×) images that focus on a single pore structure within the hydrogel. Images were acquired by Nikon on their AXR Confocal Imaging System and a z-stack reconstructed was performed;

FIGS. 19 A-E illustrate induced-diabetic keratinocytes (idKCs) exhibit decreased epidermal regeneration activity. (A) Schematic diagram of the process of inducing a diabetic-like phenotype in KCs based on prior literature, performed in parallel to healthy KCs from the same donor to allow for direct comparison if KCs and idKCs from the same donor. KCs at P1 were seeded in separate culture flasks, with 25 mM treatment resulting in induction of diabetes after 10 days. (B) Morphological images of healthy vs. idKCs. The idKCs population have an apparent shift towards more elongated-shaped cells [Scale bar=100 μm]. The functional effect of diabetes induction on epidermal activity of idKCs (teal bar) relative to healthy control KCs (black bar) was evaluated via (C) metabolic, (D) proliferative, and (E) migratory changes in the idKC populations. A representative image of the scratch assay at 24 h is provided for KC and idKCs. White lines depict original wound edge. Yellow region highlights remaining region not recovered. [Scale bar=50 μm]. Significance denoted as *p<0.05 or ***p<0.001;

FIGS. 20 A-D illustrate matrix substrates alter ASC secretion of factors that modulate epidermal regeneration functional activity in idKCs. The effect of ASC-CM from 2D (silhouette) and 3D (patterned) systems that were coated (or non-coated control) was evaluated for ability to modulate idKC (A) metabolic, (B) proliferative, and (C) migratory activity. Functional activity data are denoted as relative change to baseline control idKCs, which were cultured with keratinocyte growth media (KC-GM). Migratory data depicted as percent (%) area recovered. Dashed line depicts idKCs control. (D) qRT-PCR analysis of CCND1, CDKN2A, IL1B, EGFR, FLG, and TWIST1. GAPDH was used as an internal control. Values are represented as relative fold change to baseline control idKC expression, indicated by dashed line, using the ΔΔCt method. NC, non-coated; Col 1, collagen type I; Fn, fibronectin; Fib, fibrin. Significance denoted as *p<0.05, **p<0.01, ***p<0.001, and ****p<0.0001;

FIG. 21 illustrates augmenting the idKC epidermal phenotype via matrix-dependent modulation of ASC secretome. qRT-PCR analysis of CDH1 (E-cadherin), CDH2 (N-cadherin), K5, K10, and K16. GAPDH was used as an internal control. Values are represented as relative fold change to baseline control idKCs indicated by dashed line, using the ΔΔCt method. NC, non-coated; Col 1, collagen type I; Fn, fibronectin; Fib, fibrin. Significance denoted as *p<0.05, **p<0.01, ***p<0.001, and ****p<0.0001;

FIGS. 22 A-D illustrate matrix-coating within tissue-mimetic system enhances the relative secretion of regenerative compounds from ASCs. (A) ASC-Cm collected from each group was evaluated for total protein concentration via QuickDrop, BCA, and bradford (coomassie). The figure depicts QuickDrop data. (B) A total of twelve (12) ELISAs were performed on ASC-CM samples. Only seven (7) contained a high enough protein concentration above the limit of detection for the ELISA. Five (5) key factors of those seven (7) are depicted in the figure. (C) A table to depict the relative concentration of each protein tested (in pg″) in relation to the total amount of secreted protein (in μg″). An X″ depicts that the sample was below the limit of detection for the ELISA. (D) ASCs were evaluated for changes in gene expression of key markers (28 selected) via a Wound Healing qRT-PCR array (84 total target) and depicted with a heatmap. A fold change of >10 is denoted as the highest increase in fold change (dark blue). The markers selected are associated with secretory activity from ASCs and several align with the proteins of interest for the ELISAs. GAPDH, ACTB, and B2M were the endogenous control genes utilized by the array. NC, non-coated; Col 1, collagen type I; Fn, fibronectin; Fib, fibrin. Significance denoted as *p<0.05, **p<0.01, ***p<0.001, and ****p<0.0001;

FIGS. 23 A-F illustrate ASC exposed to collagen type I in 3D enhance epidermal regeneration of idKCs via secreted EVs. (A) ASC-EVs were isolated from 2D (silhouette) and 3D (patterned) ASC-CM and the relative concentration of EVs per sample were analyzed via protein content. (B) The relative quantity of EVs to total secreted protein was then calculated to determine relative compositional changes for each group. (C) Metabolic, (D) proliferative, (E) and migratory activity were evaluated for idKCs treated with KC-GM dosed with 150 μg/ml of EVs. (F) qRT-PCR analysis of CCND1, CDKN2A, FLG, K5, K10, and K16 was then performed to assess for expressional changes in idKCs. GAPDH was used as an internal control. Values are represented as relative fold change to baseline control idKC expression, indicated by dashed line, using the ΔΔCt method. NC, non-coated; Col 1, collagen type I; Fn, fibronectin; Fib, fibrin. Significance denoted as *p<0.05, **p<0.01, ***p<0.001, and ****p<0.0001;

FIG. 24 illustrates ASC-CM Soluble Protein Quantification. Twelve ELISAs were used to quantify specific protein compounds known to be important for epidermal regeneration natively, including EGF, HB-EGF, IGF-1, FGF-2, FGF-7 (KGF), TGF-β1, MMP-1, MMP-2, TIMP-1, TIMP-2. IL-1β, and IL-1Ra. Of the twelve, only eight had a sample above the limit of detection, seven of which were above for both 2D and 3D. MMP-9 was not detected in 2D (leftmost columns) samples but was in 3D (rightmost columns). NC=non-coated, Col 1=collagen type I, Fn=fibronectin, Fib=fibrin. Significance denoted as *p<0.05, **p<0.01, ***p<0.001, and ****p<0.0001;

FIGS. 25 A-F illustrate ASC exposed to collagen type I in 3D enhance epidermal regeneration of idKCs via secreted EVs. (A) ASC-EVs were isolated from 2D (silhouette) and 3D (patterned) ASC-CM and the relative concentration of EVs per sample were analyzed via protein content. (B) The relative quantity of EVs to total secreted protein was then calculated to determine relative compositional changes for each group. (C) Metabolic, (D) proliferative, (E) and migratory activity were evaluated for idKCs treated with KC-GM dosed with 150 μg/ml of EVs. (F) qRT-PCR analysis of CCND1, CDKN2A, FLG, K5, K10, and K16 was then performed to assess for expressional changes in idKCs. GAPDH was used as an internal control. Values are represented as relative fold change to baseline control idKC expression, indicated by dashed line, using the ΔΔCt method. NC, non-coated; Col 1, collagen type I; Fn, fibronectin; Fib, fibrin. Significance denoted as *p<0.05, **p<0.01, ***p<0.001, and ****p<0.0001;

FIG. 26 illustrates characterization of EV population within ASC-CM. The isolated EV fraction was quantified via NTA to establish particle counts for concentration. Significance denoted as *p<0.05, **p<0.01, ***p<0.001, and ****p<0.0001;

FIG. 27 illustrates quantification of relative antioxidant activity within ASC-CM. ASC-CM was collected from 2D (silhouette) and 3D (patterned) cultured systems, and antioxidant activity was assessed with a Total Antioxidant Capacity (TAC) Assay kit. In short, the reduction of copper (II) to copper (I) by antioxidant activity is assessed, with the naturally occurring antioxidant uric acid, used as a control standard for the kit. Antioxidant activity of ASC-CM was therefore measured in “mM equivalents” of uric acid.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to methods and/or platforms that may manipulate a constituent profile of a secretome composition of a cell type of interest, such as for example adipose-derived Mesenchymal Stem Cells (ASCs). In this regard, individual constituents of the secretome composition may include, for example, a variety of different growth factors, proteins, cytokines, extracellular vesicles (EVs), chemokines, hormones, digestive enzymes, antibodies, extracellular proteinases, morphogens, toxins, and antimicrobial peptides. The desire to manipulate a constituent profile of a secretome composition of a cell type of interest may be pursuant to additional aspects of the invention that may identify or characterize one or more therapeutically effective constituents amongst the constituent profile of the secretome composition. In this regard, certain embodiments of the invention may mimic in vivo cell secretion in an ex vivo setting followed by characterization of the resulting constituent profile of a secretome composition of a cell type of interest and identification of one more constituents that are believed, shown, or otherwise known to provide a therapeutic effect, which may form, at least in part, a target constituent profile of the secretome composition having increased and/or decreased quantities of one or more constituents and/or relative ratios between one or more constituents.

In this regard, certain embodiments of the invention may provide detailed protocols that can be developed using one or more tissue mimetic three-dimensional (3D) hydrogel blocks (TMHB) to externally reprogram how cells behave and which biological factors are secreted from cells. The TMHBs may comprise a macrostructure defined by a continuous polymeric matrix material and a network of microporous channels and/or chambers extending throughout the continuous polymeric matrix material with a first cell type of interest. In accordance with certain embodiments of the invention, the THMBs may be interlockable with each other to provide a 3D continuous scaffold for cell propagation and growth that more resembles a native environment for the particular cell type or types of interest. As used herein interlockable THMBs may be referred to as interlocking porous hydrogel blocks (IPHB). Certain embodiments, in accordance with of the invention, enables the development of highly specific protocols that lead to a desired outcome for producing a target biological factor or factors (e.g., a constituent(s) of a secretome composition). These protocols can then be used to scale production of the desired biological factor(s), such as for a therapeutic. Additionally, certain embodiments of the invention enables the development of protocols to specifically control the biological factor(s) secreted by different cell types cultured in TMHBs for manufacturing, for example, therapeutic agents, diagnostic markers, and prognostic markers.

TMHBs comprise hydrogels that contain a network of microchannels and/or micropods that permeate through the entire volume of the TMHB. The TMHBs may be shaped in such a way that the macrostructure is that of a jigsaw puzzle piece or similar shape that allows for interlocking of TMHBs utilizing a male-to-female connector (e.g., an IPHB). IPHBs may be interlocked together similarly to how toy Lego Blocks snap together to build a greater structure. When IPHBs are joined together, the microchannels at the surfaces thereof at least partially align, and permit fluids, gases, and cells to move between the two or more IPHBs freely.

TMHBs, whether of the interlocking variety or not, are modular. The microchannels can be made to be static or dynamic so that that the microchannels contract or dilate based on media formulation applied to the TMHB. The hydrogel, for example, can be formulated to tailor different mechanical, physical, and chemical properties for different cell types to mimic specific tissues. For example, the hydrogel formulations can be tuned to adjust stiffness and elasticity. Additionally, the shape and pattern of the microchannels can be adjusted to change liquid flow and cell migration. Additionally or alternatively, coatings, peptides, and other growth factors can be applied to the interior surface of the hydrogel (e.g., interface between the continuous polymeric matrix material and a network of microporous channels and/or chambers) to adjust the “stickiness” (e.g., affinity of a cell type of interest to adhere thereto) of the hydrogel for cell attachment. Furthermore, TMHBs of the IPHB variety can be interconnected in different configurations to control and direct cell movement and fluid flow. For example, IPHBs may be joined in a linear configuration to promote cells growing in a straight line or cells can configured in grid to promote the formation of a network of cells. IPHBs can be chained or networked together in any horizontal or vertical configuration for making custom microfluidic devices. Cells grow on the interior surfaces of microchannels, and any biological factors secreted by cells are into the luminal space. Media can easily be aspirated out of the microchannels and collected and filtered to isolate exosomes. This process allows end-users to easily collect exosomes or other biological factors for different applications. As different configurations and different types of IPHBs can be joined together, the microenvironment and stimuli applied to single cell types or multiple cell types can be readily changed to alter the profile of biological factors secreted by target cells, the quality of the biological factors secreted by target cells, and quantity of biological factors secreted by target cells. Cells respond to external stimuli and injury to secrete biological factors as a means of communication, which can easily be exploited in accordance with certain embodiments to manipulate or tailor the constituent profile of a secretome composition of a cell type of interest.

As noted throughout, the TMHBs of the IPHB variety can be joined together in multiple configurations to change the profile, quality, and quantity of biological factors, such as exosomes, that are secreted from cells cultured inside IPHBs. No other known platform can control the quality and quantity of the exosomes secreted, let alone modulate the profile of exosomes secreted. Furthermore, many of the competing platforms lack the ability to be easily tailored to different cells or stimuli to induce a change in the secretion profile of exosomes. TMHBs of the IPHB present many opportunities to manipulate how cells grow and behave within the IPHB platform. To the contrary, all other platforms focus on stimulating the mass production of exosomes, but do not consider the mechanical, physical, and chemical substrate on which the cells that produce exosomes are cultured. Most other platforms, for instance, focus on creating media formulations to stimulate exosome production while ignoring the actual format of the substrate on which cells are cultured to produce MSCs.

In this regard, parameters of TMHBs of the IPHB or not that can be easily manipulated individually or in combination to produce desired biological factor outputs include the following: hydrogel composition (e.g., biological materials and/or synthetic materials); hydrogel mechanical properties (e.g., stiffness, elasticity, ductility, viscoelasticity, compression, tension, and torsion); hydrogel structure, such as the microchannel shape or cross-section (e.g., circular, triangular, square, polygonal), microchannel starting diameter size (before swelling or exposure to cells) (e.g., 200 microns, 300 microns, 400 microns, 500 microns, 600 microns, 700 microns, 800 microns, as well as any micron value between 200 and 800 microns), uniform microchannel starting diameter size (e.g., all microchannels are same diameter), variable microchannel starting diameter size (e.g., microchannels may have differing diameters), pattern variable microchannel diameter (e.g., defined pattern of which microchannels have differing size diameters—for example—the central microchannel and most distal microchannel from the center microchannel may be of one diameter where all other microchannel diameters of a different diameter, random variable microchannel diameter (e.g., differences in microchannel diameters are randomized in which no discernable pattern in which microchannel diameters are one size and which microchannels are of a different size.), and microchannel spacing (e.g., diffusion gradients and osmotic gradients can be manipulated by increasing or decreasing the spacing between microchannels in the hydrogel; hydrogel swelling based on media composition (e.g., basal media salinity, basal media pH, basal media sugar content, basal media protein content); and hydrogel swelling physical parameters (e.g., liquid temperature, environmental pressure, environmental humidity). Additional parameters of TMHBs of the IPHB variety or not that can be easily manipulated individually or in combination to produce desired biological factor outputs include cell responses to media formulation (e.g., basal media, growth factor composition, presence of serum, presence of animal components, presence of human components, presence of antibiotics, presence of antifungals); and cell response to environmental stimuli, such as an extracellular matrix coating (animal, human, recombinant) on the hydrogel including, but not limited to, collagen, laminin, elastin, hyaluronic acid, gelatin, fibronectin, fibrin, heparan sulfate, tricalcium phosphate, and decellularized tissue). Additional parameters related to cell responses to media formulation include oxygen composition (hypoxic vs normal), pH, temperature, salinity, electromagnetic field, presence of other cell types, static fluid, moving fluid (e.g., velocity of fluid moving through microchannels, wave shape of fluid moving through microchannels, hydrostatic pressure, and duration that fluid is moving), presence of toxins in media, presence of virus in media, presence of bacteria in media, presence of yeast in media, and presence of fungi in media. Additional parameters related to cell responses to media formulation include changes to cells via physical actions (e.g., blunt instrument or exposure to sharp instrument such as a scalpel cutting through hydrogel, chemical actions like changes in pH and/or salinity and/or toxicity, mechanical actions like compression, tension, and shear stress). Additional parameters related to cell responses to media formulation include changes to cells via biological actions (including, but not limited to): introduction of one or more cell types via joined IPHB, introduction of growth factors to media, introduction of DNA, mRNA, siRNA, shRNA, microRNA, introduction of exosomes and other biological factors, introduction of proteins, introduction of growth factors, introduction of enzymes, introduction of cytokines, and introduction of peptides. Additional parameters related to cell responses to media formulation include changes to cells via electrical actions, such as electroporation, acoustic actions, and sonoporation.

In this regard, certain embodiments of the present invention provide methods and/or platforms based on the ability to control all the desired parameters simultaneously in a cell culture environment via TMHBs, whether of the IPHB variety or not.

Certain embodiments according to the invention provide methods of characterizing a therapeutically desirable constituent or constituent profile of a secretome composition secreted from a cell type of interest. The methods may comprise the following: (i) seeding a first tissue mimetic three-dimensional (3D) hydrogel block (TMHB) comprising a macrostructure defined by a continuous polymeric matrix material and a network of microporous channels and/or chambers extending throughout the continuous polymeric matrix material with a first cell type of interest; (ii) simulating in vitro cell growth and propagation of the first cell type of interest by feeding the first cell type of interest with a first culture media and allowing the first cell type of interest to propagate throughout the first TMHB and to secrete the secretome composition; (iv) collecting a secretome composition; (v) subjecting the secretome composition to a stratification operation based on molecular weight cut-off values providing a plurality of different strata each characterized by a different range of constituent-molecular weights; (vi) characterizing and/or identifying one or more individual constituents present with each strata of interest; and (v) testing each strata of interest for relative therapeutic benefit either by in vivo analysis or ex vivo analysis and identifying the strata of interest that provides a highest therapeutic benefit as the therapeutically constituent profile.

In accordance with certain embodiments of the invention, the method may comprise characterizing at least a majority (e.g., each) of the individual constituents present in the strata of interest that provides a highest therapeutic benefit. The method may also comprise a step of identifying at least one high occurrence constituent from the one or more individual constituents present in the strata of interest that provides a highest therapeutic benefit that is either not found or found in a lesser amount in each of the strata of interest to identify the at least one high occurrence constituent as the therapeutically desirable constituent.

In another aspect, the present invention provides a method of tailoring a constituent profile of a natural secretome composition secreted from a cell type of interest, comprising: (i) seeding a first tissue mimetic three-dimensional (3D) hydrogel block (TMHB) comprising a macrostructure defined by a continuous polymeric matrix material and a network of microporous channels and/or chambers extending throughout the continuous polymeric matrix material with a first cell type of interest; (ii) feeding first cell type of interest with a first culture media;
(iii) introducing at least one external stimuli configured to modify the natural secretome composition secreted from a cell type of interest to more closely track a target secretome composition (e.g., a previously identified therapeutically beneficial constituent or profile thereof) having a target constituent or target constituent profile; wherein the at least one external stimuli comprises one or more controllable parameters, such as addition of a coating on an interface between the continuous polymeric matrix material and the network of microporous channels and/or chambers, adjusting an oxygen concentration, a pH, a temperature, and fluid dynamics through the first TMHB; (iv) allowing the first cell type of interest to propagate throughout the first TMHB and to secrete the secretome composition; and (iv) collecting a modified secretome composition that is different than the natural secretome composition due to the external stimuli.

As noted above the TMHBs may be of the IPHB variety, which can be interlocked with each other to provide continuous 3D growth of a variety of cells and/or tissues. Hydrogels are insoluble polymer matrices that can be engineered to hold up to 96% water content by mass, such as up to 40, 50, 60, 70, 80, 90, and 95% water content by mass). A variety of different polymers can be used individually or in combination to create unique hydrogels. Through cross-linking of polymers via light, temperature shift, or chemical reaction, hydrogels can be tailored to exhibit different mechanical properties, diffusion gradients, osmotic pressures, chemical formulations, and structures such as pores and fibers of varying shapes and sizes. Hydrogels may also be degradable or non-degradable. Hydrogels are versatile in their ability to be used in different applications, such as soft contact lenses to provide optics to correct a patient's vision. Hydrogels have been used in wound healing applications as a dressing and have also been used as bioinks in Life Science applications to create unique structural scaffolds for micro-fluidic experiments or provide a substrate for cells to be cultured on or in.

The IPHBs, in accordance with certain embodiment of the invention, may be joined together or interlocked together via at least one interlocking-male component and at least one interlocking-female component. For example, the at least one interlocking-male component of a first IPHB is configured to be received within a corresponding at least one interlocking-female component of a second IPHB. In addition to the joining of the IPHBs via their 3D macrostructure, the microstructure of network of microporous channels and/or chambers (e.g., void spaces). Most hydrogels are solid materials. However, by introducing void spaces and microchannels into the hydrogel, liquid, gas, and cell migration can be directed for the purpose of expanding the hydrogels together to form a continuous substrate. This feature, in accordance with certain embodiments of the invention, may be particularly beneficial since microstructure of network of microporous channels and/or chambers (e.g., void spaces) allow for a second medium to be used to interlock the IPHBs (e.g., hydrogels) together, and create a larger or expanded material for cell and/or tissue growth.

Hydrogels may be formed by crosslinking any synthetic polymer, biological polymer, tissue component (derived from human, animal, plant, or combination thereof), or combination thereof in the presence of water using a free-radical mediated reaction (e.g., photo reaction, chemical reaction) or reaction as a result of change in temperature.

In accordance with certain embodiments of the invention, the IPHBs allow for cells to grow in a more native physiological-like environment compared to culture in a 2D plastic cell culture vessel. For instance, the IPHBs may be tuned or configured to mimic an original tissue environment from which specific cells arise and grow, unlike plastic cell culture vessels and other technologies that are not customizable and modular. Additionally, certain embodiments of the invention provide for the combination of multiple IPHB (e.g., hydrogel) substrates to be joined that are like or unlike to form an expanded continuous hydrogel substrate for cell production and/or biologics production. For example, like or unlike hydrogels (e.g., IPHB) may be joined together to create unique and custom microenvironments for cells to grow in, unlike other cell culture vessels or technologies.

Moreover, the IPHBs can allow for the formation of spheroids and organoids without developing a necrotic core inside the IPHB's network of microchannels. For example, the IPHBs, in accordance with certain embodiments of the invention, allows spheroids and organoids to unwind and form sheets, tubes, cylinders, and other sophisticated structures where nutrients and gases may evenly diffuse to cells within the IPHB (e.g., hydrogel). Beneficially, for instance, the IPHBs may permit even nutrient and gas exchange for healthy cell growth unlike other technologies that claim to mass produce cells. In accordance with certain embodiments of the invention, the IPHBs may be modified to suite a wide variety of different cell types. In accordance with certain embodiments of the invention, the IPHBs enables cells to secrete extracellular matrix and create natural microenvironments that promote cell growth, migration, viability, and function. Still further, the IPHBs beneficially eliminate the need to subculture cells. Moreover, the use of the IPHBs are easy to use as they can be provided in a pre-formed format, and does not require sophisticated changes in temperature, pH, or chemical exposure to use. The IPHBs, for example, may enable users to achieve one or more of the following: grow custom cell cultures, grow multiple cell types in parallel or sequence, combine cell cultures to create complex tissues, mass produce cells without ever stopping production of cells, use the same substrates to produce cells from the benchtop all the way through clinical trials and for industrial production, and use less media and fewer consumables than present technologies, reduce human error and risks of contamination by reducing or eliminating human touch points in the production of cells. In accordance with certain embodiments of the invention, the IPHBs provide a modular platform for any of the above-referenced applications.

IPHBs may comprise a three-dimensional (3D) macrostructure defined by a continuous polymeric matrix material and a network of microporous channels and/or chambers extending throughout the continuous polymeric matrix material. The 3D macrostructure may comprise a top surface, a bottom surface, and a thickness defined by at least one side edge extending from the top surface to the bottom surface, in which the 3D macrostructure includes at least one interlocking-male component and at least one interlocking-female component. In accordance with certain embodiments of the invention, the at least one interlocking-male component of a first IPHB is configured to be received within a corresponding at least one interlocking-female component of a second IPHB.

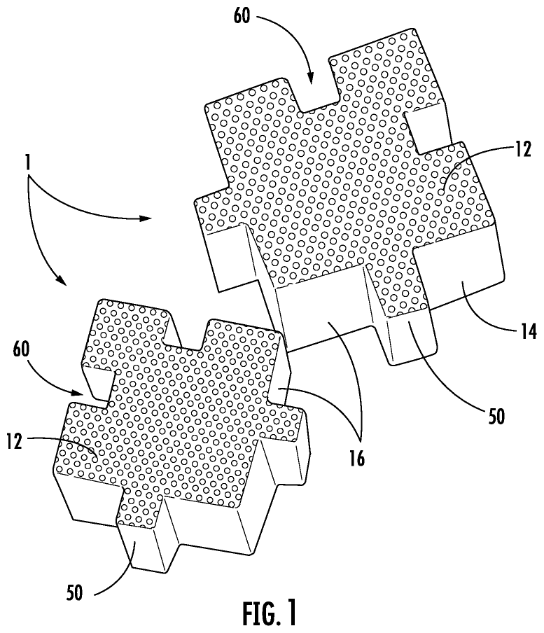

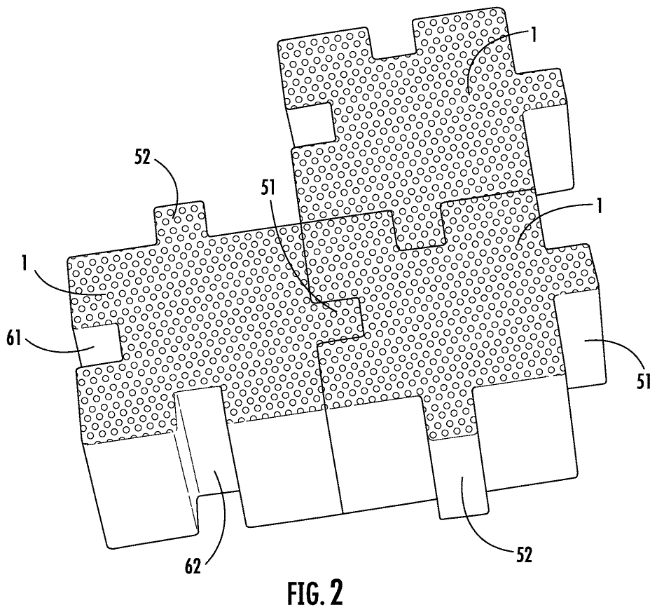

FIG. 1, for instance, illustrates two (2) separate IPHBs 1 in accordance with certain embodiments of the invention. Each of these IPHBs include a top surface 12, a bottom surface 14 and at least one side edge 16. The particular IPHBs 1 shown in FIG. 1 include at least one interlocking-male component 50 and at least one interlocking-female component 60. FIG. 2 illustrates three (3) interlocked IPHBs 1 in accordance with certain embodiments of the invention. The IPHBs 1 shown in FIG. 2 each include a first interlocking-male component 51, a second interlocking-male component 52, a first interlocking-female component 61, a second interlocking-female component 62.

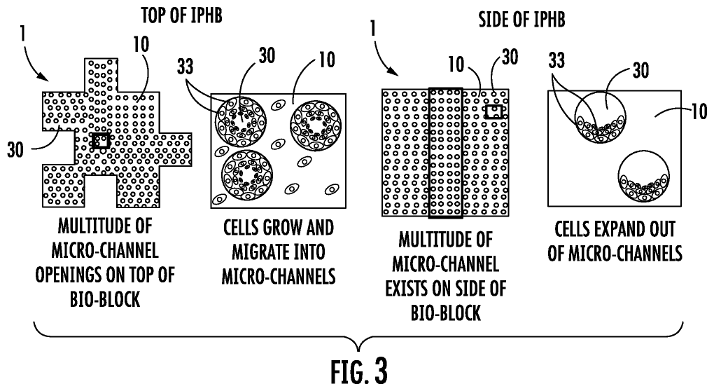

FIG. 3 depicts top and side views of an IPHB 1 and illustrates the continuous polymeric matrix material 10 and the network of microporous channels and/or chambers 30 extending throughout the continuous polymeric matrix material, which are exposed at the surfaces of the IPHB, in accordance with certain embodiments of the invention. As illustrated by FIG. 3, a seeded IPHB may enable cells 33 to grow and migrate throughout the network of microporous channels and/or chambers 30 in a three-dimensional manner.

In accordance with certain embodiments of the invention, the at least one interlocking-male component includes a first interlocking-male component extending outwardly from the at least one side edge. For example, the at least one side edge may include a first side edge and a second side edge, in which the at least one interlocking-male component includes a first interlocking-male component extending outwardly from the first side edge and a second interlocking-male component extending outwardly from the second side edge. The interlocking-male components expending outwardly from the side edges, for instance, are configured to interlock or join to corresponding interlocking-female components of other IPHBs to form an expanding continuous 3D scaffolding system with the interlocked or joined IPHBs expanding outwardly in an x-y plane. In accordance with certain embodiments of the invention, the at least one interlocking-male component may also include a third interlocking-male component extending outwardly from the top surface. In this regard, the interlocking-male components expending outwardly from the top surface, for instance, are configured to interlock or join to corresponding interlocking-female components located on a bottom surface of other IPHBs to form an expanding continuous 3D scaffolding system with the interlocked or joined IPHBs expanding in a z-direction that is perpendicular to the x-y plane. In accordance with certain embodiments of the invention, for example, a plurality of IPHBs may be interlocked or joined together in both the x-y plane and stacked upon themselves in the z-direction. As noted above, the at least one interlocking-female component may include a first interlocking-female component extending inwardly from the at least one side edge towards an interior portion of the 3D macrostructure. For example, the at least one side edge may include a third side edge and a fourth side edge, in which the at least one interlocking-female component includes a first interlocking-female component extending inwardly from the third side edge towards an interior portion of the 3D macrostructure and a second interlocking-female component extending inwardly from the fourth side edge towards an interior portion of the 3D macrostructure. In such example embodiments, the first side edge and the third side edge may define a first pair of opposing side edges, while the second side edge and the fourth side edge may define a second pair of opposing side edges. In accordance with certain embodiments of the invention and as noted above, at least one interlocking-female component may include a third interlocking-female component extending inwardly from the bottom surface towards an interior portion of the 3D macrostructure. In this regard, a plurality of IPHBs may be interlocked or joined together in both the x-y plane and stacked upon themselves in the z-direction.

In accordance with certain embodiments of the invention, the interlocking feature of the IPHBs enable the custom formation of continuous 3D scaffolds for the growth or a variety of cells and/or tissues, in which the number of particular cells being seeded and/or grown in the continuous 3D scaffold is not limited. Such flexibility in the relative positioning and interlocking of the different IPHBs enable the custom growth of multiple types of cells that may form complex interfaces between different types of cells. For example, a plurality of interlocked IPHBs defining a continuous 3D scaffold (e.g., continuous network microporous channels and/or chambers extending throughout the plurality of interlocked IPHBs) may include from 1 to 20 different cell and/or tissue types being grown simultaneously, such as at least about any of the following: 1, 2, 3, 4, 5, 6, 7, 8, 9, and 10 different cell and/or tissue types being grown simultaneously, and/or at most about any of the following: 20, 19, 18, 17, 16, 15, 14, 13, 12, 11, and 10 different cell and/or tissue types being grown simultaneously.

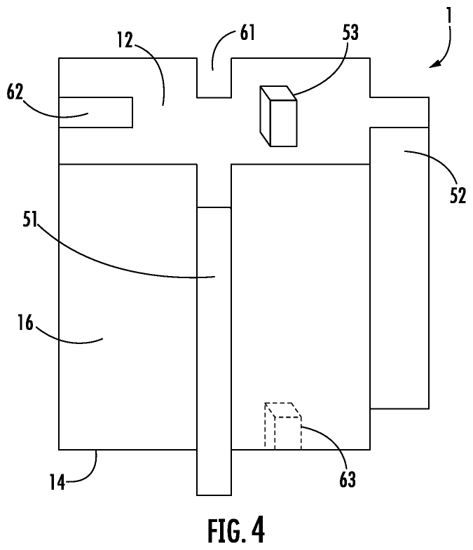

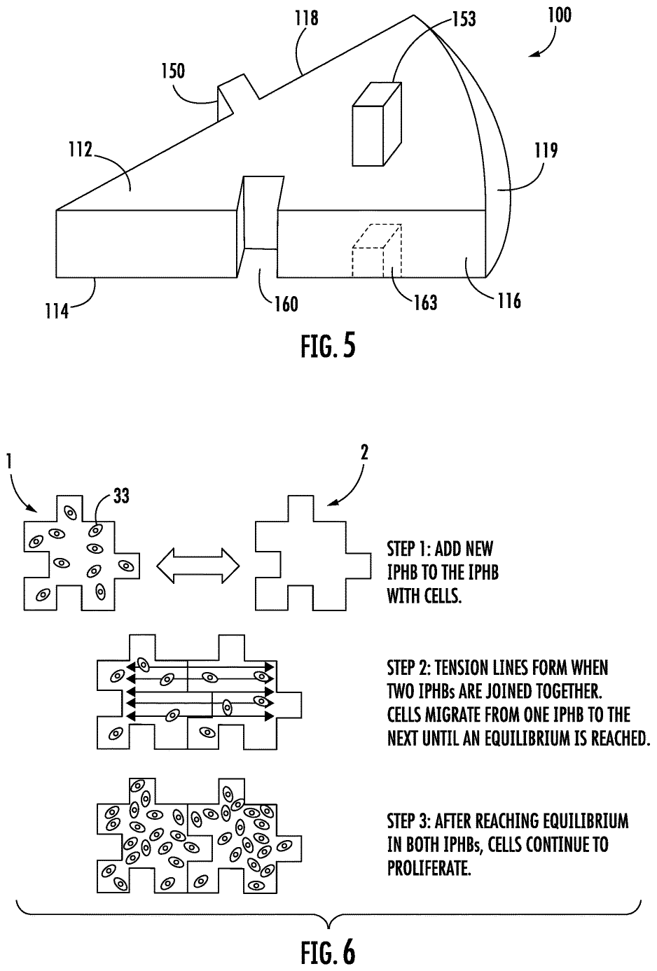

FIG. 4, for example, illustrates a IPHB 1 including an interlocking-male component 53 located on a top surface 12 and an interlocking-female component 63 protruding into the bottom surface 14 of the IPHB in accordance with certain embodiments of the invention. In this regard, the interlocking-male component 53 located on a top surface 12 and an interlocking-female component 63 protruding into the bottom surface 14 of the IPHB may be configured to engage and interlock with a separate IPHB as described herein.

In accordance with certain embodiments of the invention, the 3D macrostructure with the exception of the at least one interlocking-male component and at least one interlocking-female component may define a cube, a square prism, or a triangular prism. The 3D macrostructure with the exception of the at least one interlocking-male component and at least one interlocking-female component, in accordance with certain embodiments of the invention, define a polygonal prism having from 3 to 12 side edges, such as at least about 3, 4, 5, 6, 7, and 8 side edges, and/or at most about any of the following: 12, 11, 10, 9, and 8 side edges. In accordance with certain embodiments of the invention each side may include either an interlocking-male component and/or an interlocking-female component. Alternatively, some of the side edges may be devoid of an interlocking-male component and an interlocking-female component.

In accordance with certain embodiments of the invention, the at least one side edge includes a first side edge, a second side edge, and an arcuate side edge located between and adjacent the first side edge and the second side edge. For example, the first side edge may include the at least one interlocking-male component extending outwardly from the first side edge and the second side edge may include the at least one interlocking-female component extending inwardly from the second side edge towards an interior portion of the 3D macrostructure. In this regard, each IPHB may define a pie-like shape that when assembled or interlocked together forms a circle (e.g., cylinder since each IPHB has a thickness). Such configurations of the IPHBs may be desirable for use with circular culture wells. Additionally or alternatively, the at least one interlocking-male component includes a second interlocking-male component extending outwardly from the top surface, and/or the at least one interlocking-female component includes a second interlocking-female component extending inwardly from the bottom surface towards an interior portion of the 3D macrostructure. In this regard, a plurality of IPHBs may be stacked upon each other in a z-direction to form a thicker cylinder or semi-cylinder. In accordance with certain embodiments of the invention, the 3D macrostructure with the exception of the at least one interlocking-male component and at least one interlocking-female component may define a semi-cylinder, such as ⅛th of a cylinder to ½ of a cylinder, such as ⅛th, ¼th, ⅓rd, or ½ of a cylinder.

FIG. 5, for example, illustrates a semi-cylinder shaped IPHB 100 including an interlocking-male component 153 located on a top surface 112 and an interlocking-female component 163 protruding into the bottom surface 114 of the IPHB in accordance with certain embodiments of the invention. The semi-cylinder shaped IPHB 100 illustrated by FIG. 5 includes a first interlocking-male component 150 extending from a second side edge 118 and a first interlocking-female component 160 protruding into a second side edge 116. The first side edge 116 and the second side edge 118 of the IPHB 100 shown in FIG. 5 are connected via an arcuate side edge 119 at one end of the IPHB. In this regard, the interlocking-male component 153 located on a top surface 112 and an interlocking-female component 163 protruding into the bottom surface 114 of the IPHB may be configured to engage and interlock with a separate IPHB as described herein.

In accordance with certain embodiments of the invention, the at least one interlocking-male component may occupy or overlap from about 5% to about 50% of the macroscopic surface area of the surface (e.g., side edge, top surface, or bottom surface) upon which it extends from, such as at about any of the following: 10, 15, 20, and 25%, and/or at most about any of the following: 50, 45, 40, 35, 30, and 25%. Additionally or alternatively, the at least one interlocking-female component may occupy or overlap from about 5% to about 50% of the macroscopic surface area of the surface (e.g., side edge, top surface, or bottom surface) upon which it penetrated into, such as at about any of the following: 10, 15, 20, and 25%, and/or at most about any of the following: 50, 45, 40, 35, 30, and 25%.

In accordance with certain embodiments of the invention, the bottom surface may have a rougher texture relative to the top surface. For example, the top surface may be relatively smooth relative to the bottom surface which may have a textured structure. The textured structure at the bottom surface, for example, may facilitate the flow of a culture medium or washing medium through the entirety of the IPHB by providing structural spacers to facilitate the drainage of the culture medium or washing medium from the IPHB. The textured surface of the bottom surface, for example, may include a plurality of minor protrusions, such as individual nubs or ridges that function as short spacers. The plurality of minor protrusions, however, may be significantly smaller in size compared to the at least one interlocking-male component, such as being at most about 1/10th the size of the at least one interlocking-male component. In this regard, the minor protrusions may generally not provide any interlocking functionality in accordance with certain embodiments of the invention.

In accordance with certain embodiments of the invention, the top surface of the IPHB may comprise a macroscopic surface area from about 0.25 cm² to about 25 cm², such as at least about any of the following: 0.25, 0.5, 0.75, 1, 1.5, 2, 5, 8, 10, and 12 cm², and/or about any of the following: 25, 22, 20, 18, 15, and 12 cm². Additionally or alternatively, the bottom surface may comprise a macroscopic surface area from about 0.25 cm² to about 25 cm², such as at least about any of the following: 0.25, 0.5, 0.75, 1, 1.5, 2, 5, 8, 10, and 12 cm², and/or about any of the following: 25, 22, 20, 18, 15, and 12 cm². Additionally or alternatively, the thickness of the 3D macrostructure may be from about 0.5 cm to about 3 cm, such as at least about any of the following: 0.5, 0.75, 1, 1.25, and 1.5 cm, and/or at most about any of the following: 3, 2.5, 2, and 1.5 cm.

As noted above, the each of the at least one interlocking-female components may be configured to receive a corresponding at least one interlocking-male component of a second IPHB. In this regard, a plurality of IPHBs may be joined or interlocked together in an individually sequential addition to expand the 3D scaffold as desired along the x-y plane and alone the z-direction. For example, cell growth in a plurality of interconnected IPHBs may be continued in the z-direction by stacking layers of additional IPHBs on top of a first layer of IPHBs.

In accordance with certain embodiments of the invention, the continuous polymeric matrix material may be non-degradable. In this regard, the cells and/or tissue produced in the IPHB may need to be flushed out of the interior network of the network of microporous channels and/or chambers for further analysis, purification, or development. Additionally or alternatively, the continuous polymeric matrix material may be selectably degradable. For example, hydrogel formulations may be rendered biodegradable, such as by insertion of enzyme-sensitive sequences or utilization of native matrix-derived compounds. For example, the continuous polymeric matrix material may comprises a selectably degradable hydrogel material comprising one or more degradable polymers, such as one or more biopolymers derived from a living organism. The one or more biopolymers derived from a living organism, for example, may comprise a polynucleotide, polysaccharide, polypeptide, or any combination thereof. In accordance with certain embodiments of the invention, the one or more biopolymers may comprise collagen, gelatin, laminin, alginate, glycosaminoglycans, oligonucleotides (e.g., DNA, RNA), carbohydrates, lipids, cellulose, alginate, and proteins that can be gently and degraded, such as with the use of protein specific enzymes, ionic solvents, neutral detergents, weak acids, and peroxides to disrupt the biopolymer chains. In accordance with certain embodiments of the invention, the one or more biopolymers may comprise degradable monomers comprising esters, such as hydroxybutyrate, lactic acid, glycolic acid, and caprolactone; anhydrides, such as adipic acid, and sebacic acid; saccharides, such as cellulose, alginate, pectin, dextrin, chitosan, hyaluronan, Chondroitin sulfate, and heparin; proteins; nucleotides (DNA, RNA); peptides, such as collagen, gelatin, silk, and fibrin; urethanes; phosphates; carbonates; and vinyl chlorides. In accordance with certain embodiments of the invention, the selectably degradable hydrogel material may further comprise a synthetic polymer, such as a polyester, a polyanhydride, a polycarbonate, a polyurethane, a polyphosphate or combinations thereof. The continuous polymeric matrix material, in accordance with certain embodiments of the invention, may comprise a 3D cross-linked polymer network, a non-crosslinked polymer network, or a combination thereof.

The continuous polymeric matrix material, as noted above, may comprise a swellable hydrogel material. The swellable hydrogel material may comprise a radically mediated reaction product of at least a first monomer including an acrylate or methacrylate functional groups and a second monomer or oligomer including at least two (2) free-radically polymerizable functional groups. For example, the at least two (2) free-radically polymerizable functional groups may independently from each other comprise an acrylate or methacrylate group, an allylic group, an alkynyl, a vinyl nitrile, a vinyl ether, a vinyl ester, a vinyl amide, a styrenic group, a maleate group, a fumarate group, or a norbornene group. In accordance with certain embodiments of the invention, at least one of the first monomer or the second monomer may comprise polyethylene glycol functionality (e.g., —O(C₂H₄O)ₙH; where n has a value from 1 to 100, polypropylene glycol functionality (e.g., —O(C₃H₆O)ₙH; where n has a value from 1 to 100, and/or glycerol functionality incorporated into a backbone of the monomer and/or grafted onto the monomer as a side-chain or a component of a side chain. By way of example only, the at least one of the first monomer or second monomer comprises 2-Hydroxyethyl acrylate (HEA), Poly(ethylene glycol) methyl ether acrylate (MPEGA), N-Methylacetamide (NMA), or Poly(ethylene glycol) diacrylate (PEGDA). In accordance with certain embodiments of the invention, non-limiting examples of non-degradable monomers that may be utilized in the hydrogel materials may include polyolefins (e.g., ethylene, propylene), styrene, nylon (e.g., amides), and/or acrylics. In accordance with certain embodiments of the invention, non-limiting examples of degradable monomers that may be utilized in the hydrogel materials may include esters (e.g., hydroxybutyrate, lactic acid, glycolic acid, caprolactone), anhydrides (e.g., adipic acid, sebacic acid) saccharides (e.g., cellulose, alginate, pectin, dextrin, chitosan, hyaluronan, Chondroitin sulfate, heparin), proteins, nucleotides (e.g., DNA, RNA), peptides (e.g., collagen, gelatin, silk, fibrin), urethanes, phosphates, carbonates, and vinyl chlorides. Additionally or alternatively, a third monomer comprising a cross-linking agent may incorporated continuous polymeric matrix material. Additionally or alternatively, the swellable hydrogel material may comprise one or more natural polymers, such as plant-derived polymers (e.g., cellulosic-polymers) and animal-derived polymers.

In accordance with certain embodiments of the invention, the continuous polymeric matrix material may mimic a natural tissue of interest (e.g., a tissue of interest from a mammal) by including one or more physical properties within about 20%, such as within about 15%, 10%, 8%, 5%, 3%, or 1%, of the natural tissue of interest, wherein the one or more physical property of interest includes softness, internal texture, porosity configuration (e.g., microchannel sizes and geometric distribution), swellability, and tension. For example, the one or more physical properties may comprise an elastic and/or compressive modulus, a storage modulus at 1 Hz, loss of modulus at 1 Hz, and/or protein/chemical coating (e.g., Collagen Type I, II, III, IV, Laminin I, II, Hyaluronan, Gelatin, Fibrin, Fibronectin, etc.). By way of example only, native adipose tissue has a storage modulus at 1 Hz from 50-100 kPa, a loss of modulus at 1 Hz of 10-20 kPa, and an elastic and/or compressive modulus of 3 kPa. In this regard, for example, a IPHB may have a storage modulus at 1 Hz of about 110 kPa, a loss of modulus at 1 Hz of about 22 kPa, and an elastic and/or compressive modulus of about 3 kPa. For example, the particular chemical constituents and/or degree of crosslinking may be altered to tailor one or more physical and/or mechanical properties of the resulting continuous polymeric matrix material to mimic or mirror those associated with a natural tissue of interest. Additionally or alternatively, the surface topography/texture of the network of microporous channels and/or chambers and/or the outside of the IPHBs can manipulated. Most of these surfaces may be smooth, grooves, bumps, mounds, divots, and other surface irregularities may be introduced to alter the flow of liquid or gas through the network of microporous channels and/or chambers. Such surface irregularities, for example, may introduce turbulence to help slow the flow of liquids or gases throughout the network of microporous channels and/or chambers. By way of example only, the surface irregularities may be significantly smaller in size compared to the average diameter of the network of microporous channels and/or chambers, such as being at most about ¼th to about 1/10th the size of the average diameter of the network of microporous channels and/or chambers.

In accordance with certain embodiments of the invention, the continuous polymeric matrix material is formed via an additive manufacturing technique, such as 3D printing or digital light synthesis printing. In this regard, the network of microporous channels and/or chambers is structured to mimic the morphology of a natural tissue of interest, such as by varying the geometry and dimensions of the network of microporous channels and/or chambers to mirror the morphology of the natural tissue of interest. For instance, the morphology of a natural tissue of interest may be readily ascertained by one of skill in the art, and this morphology may be duplicated via a 3D printing or digital light synthesis printing operation to form an IPHB having a network of microporous channels and/or chambers that mimics the morphology of the natural tissue of interest.

In accordance with certain embodiments of the invention, the average diameter of the network of microporous channels and/or chambers may comprise from about 100 to about 800 microns, such as at least about any of the following: 100, 120, 150, 180, 200, 220, and 250 microns, and/or at most about any of the following: 800, 780, 750, 720, 700, 680, 650, 620, 600, 580, 550, 520, 500, 480, 450, 420, 400, 380, 350, 320, 300, 280, and 250 microns. Additionally or alternatively, the network of microporous channels and/or chambers may comprise at least about 40% by volume of the 3D macrostructure, such as from at least about any of the following: 40, 50, 60, and 70% by volume of the 3D macrostructure, and/or at most about any of the following: 90, 85, 80, 75, and 70% by volume of the 3D macrostructure.

In accordance with certain embodiments of the invention, an interface between the network of microporous channels and/or chambers and continuous polymeric matrix material (whether the IPHB variety of TMHB or not) may comprises a coating of a compatibilizer selected to promote adhesion of a primary cell of interest. This coating may be applied subsequent to formation of the IPHB. By way of example, the coating comprising the compatibilizer may comprise a biological coating including, for example, Collagen I (e.g., Human Mesenchymal Stem Cells [from Adipose, Bone Marrow, Umbilical Cord], Human Neonatal Dermal Fibroblasts, Human Adult Dermal Fibroblasts, Human Keratinocytes, Human Myocytes, Human Osteoblasts, Human Osteocytes, Human Chondrocytes, Bovine Myocytes, Porcine Hepatocytes, Porcine Chondrocytes, Porcine Osteocytes, Equine Muscle Derived Stem Cells); Laminin I (e.g., Human Induced Pluripotent Stem Cells, Mouse Dorsal Root Ganglia); Hyaluronan (e.g., Porcine Hepatocytes, Human Dermal Adult Fibroblasts); Gelatin (e.g., Human Mesenchymal Stem Cells [from Adipose, Bone Marrow, Umbilical Cord], Human Neonatal Dermal Fibroblasts, Human Adult Dermal Fibroblasts, Human Keratinocytes, Human Myocytes, Human Osteoblasts, Human Osteocytes, Human Chondrocytes, Human T cells (CD8+), Human T cells (CD4+), Human Macrophages, Bovine Myocytes, Porcine Hepatocytes, Porcine Chondrocytes, Porcine Osteocytes, Equine Muscle Derived Stem Cells); Fibrin (e.g., Human Keratinocytes); Fibronectin (e.g., human Mesenchymal Stem Cells [from Adipose, Bone Marrow, Umbilical Cord], Human Neonatal Dermal Fibroblasts, Human Adult Dermal Fibroblasts, Human Keratinocytes, Human Osteoblasts, Human Osteocytes, Human Chondrocytes); or any combinations thereof.

In another aspect, the present invention provides a scaffolding system comprising a plurality of IPHBs, such as those described and disclosed herein. In accordance with certain embodiments of the invention, for instance, the plurality of IPHBs includes a first IPHB including a first interlocking-male component and a second IPHB including a second interlocking-female component, in which the second interlocking-female component is configured to receive the first interlocking-male component. In accordance with certain embodiments of the invention, for example, the first interlocking-male component may be inserted into the second interlocking-female component, in which the first IPHB and the second IPHB are each provided in a swollen state thereby improving interlocking of the first IPHB and the second IPHB. In this regard, the swollen state may be provided due to the absorbance of a liquid, such as water or a culture medium. As the first and second IPHBs swell during an interlocked state, the frictional forces between the mated interlocking-male and interlocking-female component(s) increases to increase the force required to separate the IPHBs.

In accordance with certain embodiments of the invention, the first IPHB has a first network of microporous channels and/or chambers and the second IPHB has a second network of microporous channels and/or chambers, in which a first portion of the first network of microporous channels and/or chambers at least partially overlaps with a first portion of the second network of microporous channels and/or chambers when the first IPHB and the second IPHB are interlocked and define an aggregate continuous network of microporous channels and/or chambers. In this regard, the density of microporous channels at the surfaces of the IPHBs is sufficiently large, as noted above, such that partial overlap of a least portions of the microporous channels at the surfaces of the respective IPHBs enables formation of a continuous aggregate continuous network of microporous channels and/or chambers extending throughout the each IPHB that may be interlocked. As noted above, a plurality of IPHBs may be interlocked together along that x-y plane and/or along the z-direction.

In accordance with certain embodiments of the invention, the first IPHB may be seeded with a first primary cell and the second IPHB is seeded with a second primary cell, wherein the first primary cell is different than the second primary cell. As noted above, each of the IPHBs may be seeded by a different and/or unique primary cell or a combination of a plurality of primary cells. Alternatively, for mass production of a given cell of interest, each IPHB may be seeded with the same primary cell.

By way of example only, one or more of the TMHBs (whether the IPHB variety or not) may be seeded and enable cell growth of any of the following: (1) Human Stem Cells, such as Human Wharton's Jelly Cells (MSC), Human Bone Marrow Derived Mesenchymal Stem Cells (MSC), Human Adipose Derived Mesenchymal Stem Cells (MSC), Human Skin Derived Induced Pluripotent Stem Cells (iPSC), Human Blood Cell Derived Induced Pluripotent Stem Cells (iPSCs), Human CD4+ T Cells, Human CD8+ T Cells; (2) Primary Mammalian Cells, such as HepG2 Cells (Liver Carcinoma Cells), Human Adult Dermal Fibroblasts (Primary Cells), Human Neonatal Dermal Fibroblasts (Primary Cells), Human Adult Keratinocytes (Primary Cells), Mouse Dorsal Root Ganglia (Primary Neural Cells), Bovine Myocytes (Primary Cell Line), Primary Porcine Hepatocytes, Porcine Chondrocytes, Porcine Osteocytes, Equine Muscle Derived Stem Cells (Primary MSCs), Primary Snail Cells, Human Macrophages; (3) Immortalized Mammalian Cell Lines, such as UB-OC2 Cells (Mouse Cochlear Epithelium), Human Myoblastoma (Muscle Tumor), PC3 (Prostate Cancer), CHO (Chinese Hamster Ovary Cell), HEK293 (Human Embryonic Kidney Cell), SHSY5Y (Neuronal Tumor), PANC-1 (Human Pancreatic Cancer), HeLa (Cervical Cancer), A549 (Lung Cancer), A673 (Muscle Cancer); and (4) Primary Plant Cells, such as Rosemary, Tobacco, and Tomato.

FIG. 6 illustrates cell 33 migration from a first IPHB 1 to a second IPHB 2 when interlocked together in accordance with certain embodiments of the invention. As illustrated in FIG. 6, the first IPHB 1 may be seeded with cells 33 and then joined to an empty (e.g., devoid of cells) second IPHB 2. Once joined together, the cells 33 migrate from the first IPHB 1 into the second IPHB 2 until an equilibrium is reached. After reaching equilibrium in both IPHBs, the cells 33 will continue to proliferate.

### WORKING EXAMPLES

The present disclosure is further illustrated by the following examples, which in no way should be construed as being limiting. That is, the specific features described in the following examples are merely illustrative and not limiting.

### Example Set #1: Secretome Stratification and Tissue-Mimetic System Identify Key Role of Exosomes in Augmentin Epidermal Regeneration

### Background and Executive Summary

Recent investigations demonstrate that the secretome of Adipose-derived Mesenchymal Stem Cells (ASCs) offers a unique approach to understanding and treating wounds, including the critical process of re-epithelialization (epidermal regeneration) orchestrated by keratinocytes. However, 2D culture techniques drastically alter the secretory dynamics of ASCs, which has led to ambiguity in understanding which secreted compounds (e.g., growth factors, cytokines, exosomes, ROS) may be driving re-epithelialization processes. In this working example a tissue-mimetic 3D hydrogel system (e.g., TMHB system) is utilized to enhance the retainment of a regenerative ASC phenotype and provide an opportunity to highlight the secretome differences between 2D and 3D culture. Culture of ASCs within the tissue-mimetic system enhanced the secretion of protein~50%. Subsequently, the ASC-secretome was stratified by molecular weight and the presence/absence of extracellular vesicles (EVs). This approach revealed that the capacity for the ASC-secretome to modulate re-epithelialization functions, including migration, proliferation, differentiation, and morphology, resided within the “>100 kDa” fraction, with the 3D ASC-secretome providing the greatest improvement. ASC-EV secretion was enhanced 2-fold in 3D and demonstrated dose-dependent effects on several key re-epithelialization functions. Notably, ASC-EVs induced substantial morphological changes in keratinocytes reminiscent of native wound healing, including formation of stratified cell sheets. However, only 3D-EVs promoted collective cell sheet migration, whereas 2D-EVs contained an anti-migratory stimulus. Similarly, 3D-EVs provided a more effective stimulus for an epithelial-to-mesenchymal transition in keratinocytes. This working example demonstrates how critical culture environment is on influencing ASC-secretome regenerative capacity. Additionally, the key role of EVs in modulating epidermal regeneration is revealed and their translatability for future clinical therapies is discussed.

### 1. Introduction

Skin is the largest organ of the human body and provides a crucial external barrier for our body that helps prevent desiccation and infection, while also offering protection from mechanical, ultraviolet, and physical insults. Repair and regeneration of the skin is a dynamic series of events that occurs in the setting of tissue damage and involves a diverse array of interrelated cell populations that communicate via mechanical, physical, and biochemical cues. Secreted paracrine compounds from resident cell populations maintain a key role in orchestrating proper cellular and molecular signaling pathways during the wound healing process. Ultimately, the goal of physiological wound healing is to promote the migration and proliferation of appropriate cells into the wound environment, stimulate neotissue formation via deposition/remodeling of extracellular matrix (ECM) components, and modulate cellular biophysical and biomechanical constructs (e.g., cytoskeletal rearrangements to promote migration and/or wound contraction).

During physiological wound healing, cells undergo phenotypic changes to increase their regenerative capabilities in an attempt to restore anatomical homeostasis. Notably, a fundamental step of proper wound healing is sufficient “closure” of the wound, also known as re-epithelialization, which helps protect the wound and deeper tissue structures. The process of re-epithelialization is performed by epidermal keratinocytes, and inadequate keratinocyte activity will result in protraction of wound closure and an increased risk for adverse outcomes such as chronic wounds, surgical interventions, need for amputations, and an overall increase in patient morbidity. Proper keratinocyte re-epithelialization relies on keratinocytes modulating their cytoskeletal and junctional proteins to become more migratory and/or proliferative. Paracrine activity from surrounding cell populations in the epidermis, dermis, hair follicles and subcutaneous tissue play a key role in orchestrating this phenotypic switch of keratinocytes. However, there remains a critical need to develop therapies aimed at augmenting appropriate keratinocyte functionality to prevent progression of chronic wounds.

Recent investigations into stem cell derived therapies have emerged as possible avenues for producing future regenerative treatments for wounds. More specifically, mesenchymal stem/stromal cells (MSCs) are multipotent progenitor cells that are found in a variety of tissue sources within the human body (including dermal adipose and hair follicles) and contain intrinsic regenerative capabilities due to the compositional plasticity of their secretory profile. Recent research has demonstrated that MSCs can exhibit diverse therapeutic capabilities for enhancing wound healing via modulation of a number of tissue regenerative processes. Notably, recent studies suggest that the adaptive secretory nature of MSCs, rather than their multipotent nature, may be driving many of the regenerative effects seen in prior in vitro and in vivo studies. Thus, MSC-derived acellular byproducts offer a cell-free alternative to current cell-based therapies and have shown the capacity to modulate wound healing activity, including the augmentation of keratinocyte re-epithelialization activity.

Interestingly, prior clinical studies investigating the utility of biological compounds, such as growth factors or cytokines, have demonstrated mixed results. However, this is likely due to the more complex nature of wounds (especially chronic wounds) that require a heterogenous milieu of factors that balance one another rather than the presence/absence of a single compound (e.g., single growth factor). Additionally, a growing body of evidence suggests extracellular vesicles (EVs), such as exosomes, are more stable and may play a key role in native tissue signaling and regeneration. EVs consist of vesicle-like particles released from cells and include apoptotic bodies, microvesicles (MVs), and exosomes. EV activity and quality is dependent on the dynamic array of biomodulatory compounds found within them, which can change depending on external stimuli and cellular phenotype. Notably, the secretome of adipose-derived MSCs (ASCs) has demonstrated the ability to enhance the rate of wound healing and tissue regeneration in vivo. However, to date, it is still not clear what fractions of MSC-derived secretory factors are driving which regenerative wound healing activities, such as augmented keratinocyte migratory and proliferative activity, seen in prior studies. This is in part, likely due to the inconsistent and inefficient utilization of standard 2D culture modalities that result in MSC populations with variable phenotypes and regenerative capabilities, as well as the complex and diverse nature of the MSC secretome. Thus, investigations into more robust tissue-mimetic 3D systems (e.g., TMHBs) could provide a platform to standardize MSC culture and generate more rigorous data pertaining to the regenerative mechanisms of MSC-derived biologics and provide an opportunity to enhance and/or tailor their regenerative capabilities.

It is important to understand that our body's native tissue microenvironments provide a range of biomechanical and biophysical cues that regulate cellular fate and behavior within that environmental niche. Native 3D tissue environments typically exhibit a range of viscoelastic properties that affect mechanotransductive and biochemical pathways. MSCs have been shown to be highly dependent upon these 3D interactions, including the role of focal adhesive sites, cell-to-cell interfaces, applied forces, and matrix stiffness, to maintain their “stem-like” phenotype and regenerative capabilities. Thus, the native MSC niche is more accurately represented by in vitro 3D systems rather than 2D, which can improve the efficacy and reproducibility of future regenerative therapies and provide a more appropriate understanding of physiological signaling. Notably, most current 3D hydrogel systems (e.g., TMHB systems) rely on manipulating densely-packed crosslinked networks in order to modulate the mechanical properties of the hydrogels, which drastically limits the ability for molecules to readily diffuse throughout the system. However, the novel microarchitectural design of the 3D hydrogel system (e.g., TMHB systems) in this study acts as a “pseudo-vasculature” conduit and does not hinder mass transport like traditional poured/molded hydrogel systems, which improved the collection of secreted byproducts, in addition to adequate nutrient exchange and prevention of centralized necrosis within the hydrogel.

Barriers related to the translatability of cell-based regenerative therapies, such as depleted regenerative capabilities of unhealthy autologous populations and inadequate ex vivo expansion systems, have led to a rising interest in regenerative acellular therapies. While previous data with MSC-derived acellular products are promising, experiments using MSCs in wound healing to date have mostly utilized standard 2D culture techniques, which are known to induce differentiation and senescence. Thus, traditional 2D culture modalities likely result in a heterogeneous and less regenerative MSC populations, leading to impurities and/or an inconsistent secretive product that subsequently limits the potential clinical benefits of MSC therapies. As a result, there is a growing interest in developing more efficient 3D expansion systems.

In this study, we utilize a tissue-mimetic 3D hydrogel system (e.g., TMHB systems) that is mechanically analogous to native adipose tissue for the culture of ASCs (Table 1 below).

TABLE 1

| | Storage | | | “Secant” |
|---|---|---|---|---|
| | modulus | Loss | | modulus |
| Frequency | (E [symbol] ) kPa | modulus | TanDelta | (E) (at plastic |
| obtained | 1 Hz | (E [symbol] ) kPa | (E [symbol] /E [symbol] ) | deformation) |
| Bio-block | 48.5 | 4.5 | 0.1 | 8.4 |
| hydrogel system | | | | |
| Native adipose | 10-100 | 1-20 | 0.1-0.2 | 1-10 |
| (based on | | | | |
| literature) | | | | |

[symbol]

indicates data missing or illegible when filed

We have previously published the benefits of this novel system in protecting ASC populations from induction of senescence and loss of a “stem-like” phenotype, subsequently resulting in a more regenerative population capable of modulating wound healing activity. An additional benefit of this system is the unique microarchitectural design that does not hinder mass transport and permits the efficient collection of secreted byproducts. Thus, this system acts like a “bioreactor” for generating biological byproducts from cell populations. Within this study, we stratify ASC conditioned media (ASC-CM) based on molecular weight cutoffs and EV/exosome particle content in order to compare and identify which fractions of ASC-CM are driving specific wound healing activities previously seen in keratinocytes. Uniquely, we will compare and contrast the effect of ASC-CM from 2D versus 3D. We hypothesized that the culture conditions of our tissue-mimetic system would be a more effective measure of how ASCs respond natively and identify functional differences between the ASC-CM from 2D and 3D. Moreover, we suspect that the exosome fraction of “stem-like” populations, such as ASCs, are critical in driving many of the functionals benefits seen in keratinocyte wound healing activity from prior studies.

### 2. Materials and Methods

### 2.1. Cell Culture

Human adipose-derived mesenchymal stem cells (ASCs; Lonza, Lot #18TL212639, 23-year-old Female, Black), human keratinocytes (KCs; Lonza, Lot #18TL318559, 62-year-old Male, Caucasian), were utilized in this study. ASCs were cultured in RoosterNourish MSC-XF (RoosterBio; Cat. #KT-016) for growth media (MSC-GM) and switched to RoosterCollect EV-Pro (RoosterBio; Cat. #K41001) for serum-free, low particulate media. DermaLife K Keratinocyte Medium Complete Kit was obtained from Lifeline Cell Technologies (Maryland, USA; #LL-0007) and used for KC culture.

### 2.2. Three-Dimensional (3D) Printed Hydrogel Cell Culture System

The 3D hydrogel system (e.g., TMHB system) is ~1-cm³ and is a bioprinted cell culture and expansion system called an X-Block (Ronawk; Kansas, USA) that contains a unique macro- and micro-architectural design that permits mass transport and nutrient exchange. The hydrogel is printed utilizing a pre-defined and specific microstructure that results in the creation of voided/porous regions that create continuous microchannels with 300-lm diameters. The microporous component makes up 44% of the total volume of the hydrogel. Additionally,

the integrated microarchitectural design significantly increases the surface area-to-volume ratio to enhance cellular proliferation and migration. The X-Block hydrogels, (Ronawk Inc.) are fabricated with a proprietary mixture of biodegradable substrates that contain biologically native binding epitopes for cellular attachment. The 3D hydrogels were placed into a glass 6-well culture plate for culturing. Cells were then added dropwise to the surface of the hydrogels and allowed to migrate into the microarchitecture over the span of 15-min, followed by submersion of the hydrogels with culture media. A thin coating of the bioink was utilized for the 2D culture control to account for any potential role of the substrate effects in 3D.

### 2.3. Mechanical Properties of Tissue-Mimetic Hydrogel

A subset of 3D hydrogels were bioprinted at a z-height of 1.2-mm (thickness) while still maintaining all other dimensional and structural characteristics of the unique architectural design of the full-sized hydrogels. Hydrogels were analyzed with a Dynamic Mechanical Analyzer (RSA3; TA Instruments) setup to assess mechanical and viscoelastic properties (Table 1). A 5-mm biopsy punch was used to isolate a circular hydrogel sample to prevent force-concentrating points. Two (2) 5-mm punches were taken from each hydrogel and a total of four (4) hydrogel were evaluated for a total of eight (8) runs (n=4). DMA was performed via a dynamic cylindrical compression analysis with a rate of compression of 0.005-mm/sec, in addition to a frequency sweep analysis over a range of 0.1-10 Hz. The frequency sweep analysis helps evaluate the viscoelastic properties of a given material by determining the relationship between a given compression frequency range and the storage (E0) and loss (E00) moduli of a material. The compression frequency will gradually increase and the viscoelastic response at each given frequency is recorded. Expression of the viscoelastic moduli at 1 Hz is generally depicted due to the similarities of this frequency range and most soft tissues within physiological conditions, including human adipose.

### 2.4. Expansion of ASCs and KCs

“Passage 1 (P1)” ASCs and KCs were seeded on 2D plastic and cultured until ~80% confluency before subculturing (i.e. passaging). Subculturing of cells was performed by removing culture media, washing 3×, and incubating with 0.05% Trypsin/EDTA (Lonza; Cat. #CC-3232) at 37° C. for 5-minutes. Trypsin was neutralized with serum-based media and cells were centrifuged at 500 g for 5-minutes, pelleted, and resuspended for reseeding on new 2D tissue culture plastic vessels or for use in experimental assays. The increased surface area of a single 3D hydrogel eliminated the need for subculturing for the time course of this study (please see prior study details), therefore 2D and 3D cells were seeded at the same seeding density of ~1,500 cells/cm² to allow for analogous comparison between 2D and 3D culture. KCs were seeded at density of ~7500 cells/cm² for experimental assays. After an initial characterization of P1 ASCs, subcultured ASCs at P2 were seeded into 3D or re-plated in 2D and cultured for 1-week and then analyzed or subcultured. 2D ASCs that were subcultured at 1-week were then re-plated for an additional week to obtain data for the P3 (2-week) data, whereas 3D ASCs were allowed to continuously culture within the 3D system without subculture (P3-equivalent/2-week) (n=4).

### 2.5. Assessment of ASC Phenotype

Initial assessment of adipogenic, chondrogenic, and osteogenic trilineage differentiation potential of ASCs (at P1) was performed via culture with differentiating media, according to the manufacturer's instructions and as previously described. Adipogenic differentiation was performed using hMSC Adipogenic Differentiation BulletKit™ (Lonza; Cat. #PT-3004). Chondrogenic differentiation was performed using hMSC Chondrogenic Differentiation Medium BulletKit™ (Lonza; Cat. #PT-3003), and was supplemented with TGF-03 (Lonza; PT-4124) at a concentration of 10 ng/mL. Osteogenic differentiation was performed using hMSC Osteogenic Differentiation Medium BulletKit™ (Lonza Cat. #PT-3002). All primary antibodies were obtained from Abcam (Cambridge, UK) unless otherwise stated. Evaluation of ASC “stem-like” phenotype was performed with the initial population at P1 via positive immunolabeling for CD73/90/105, as previously described. In brief, ASCs at P1 were seeded in 2D, fixed with 4% paraformaldehyde, washed 3×, blocked with 1% donkey-serum, and immunolabeled for CD34 (ab81289), CD45 (ab40763), CD90 (ab181469), CD105 (ab231774). CD73 (Cat. #41-0200) was obtained from Invitrogen (Waltham, MA). Cells were counterstained with an immunofluorescent nuclear marker, Hoechst 33342 (Invitrogen; Cat. #H₃₅₇₀) and for some samples Alexa Fluor 488 Phalloidin (ThermoFisher; Cat. #A12379). Additionally, ASCs from P1 (baseline control), P2 (1-week), and P3 (2-week) for 2D culture and P2 ASCs from 3D that were cultured for 1-week (P2-equivalent) or 2-weeks (P3-equivalent) were assessed via an RT² Profiler™ PCR Array for Human Mesenchymal Stem Cells (Qiagen; Cat. #330231; PAHS-082ZC-24) to evaluate expression of 84 MSC and MSC-associated genes. RNA was isolated and purified via an RNeasy Mini Kit (Qiagen). Only RNA with a 260/280 ratio of >1.8 were used for this study. Cycle threshold (Ct) values were recorded and analyzed via the Delta-Delta-Ct method. Glyceraldehyde 3-phosphate dehydrogenase (GAPDH), Beta-actin (ACTB), and Beta-2-Microglobulin (B2M) were the endogenous control genes utilized by the array (n=3).

### 2.6. Functional Characterization of Cells Via Plate Reader Assays

Both ASCs and KCs were evaluated with plate readers assays. Assays were carried out per manufacturer's instructions and as previously described. In short, plated cells were analyzed via PicoGreen fluorescence obtained at 435/535 nm (n=4) to quantify DNA as a surrogate measurement of proliferation. PrestoBlue fluorescence was obtained at fluorescence was obtained at 560/590 nm (n=4) and displayed as an average relative fluorescent unit (R.F.U.) of PrestoBlue per Hoechst signal (350/460 nm) to obtain approximate metabolic activity per cell. Similarly, MitoTracker™ Red CMXRos (Invitrogen; Cat #M7512; 500 nM) fluorescence at 570/605 nm (n=4) was used to assess mitochondrial activity/membrane potential. Lastly, population doubling rate (PDR) of ASCS extracted from 2D or 3D and then re-plated into 2D (n=5) were assessed via obtaining PicoGreen cell numbers at Day 1 and Day 4 and the formula [(t)/((log(n/nₒ))×3.32], where t=time, n=cell number at final timepoint, nₒ=initial cell number.

### 2.7. Assessment of ASC Proliferation and Viability in 2D and 3D

To evaluate the proliferation of ASCs within the hydrogel system, 2D and 3D systems were seeded with the same seeding density and cell numbers were evaluated at 1-week or 2-weeks. For 2D samples at 2-weeks, ASCs underwent one additional passaging event. At each respective time point ASCs were detached (2D) or isolated via hydrogel dissolution (3D) and replated into 2D culture overnight (~16 hours) and assessed for reattachment, cell number (PicoGreen), metabolic activity (PrestoBlue), and mitochondrial activity (MitoTracker).

### 2.8. Isolation of ASC Conditioned Media

When conditioned medium (CM) from ASC culture was desired, MSC-GM media was removed, cells were washed 3× and serum-free MSC media was added for an additional 24-hr wash. The 24-hr wash was removed and new serum-free media was added followed by collections at 24-hr intervals for three consecutive days during days 6-8. Due to variability in 2D versus 3D proliferation rates, collection days were based on previously determined cell proliferation data to establish ratio of media/cell in order to standardize media consumption. 2D confluency was in the range of 60-80% and ASC-CM was only collected from the 1-week timepoint prior to any additional subculturing events in 2D. Collected ASC-CM was centrifuged at 1500 g for 10-minutes to eliminate cell debris, Steriflip filtered with a 0.22-μm filter, and stored at −80° C. for long-term storage until use. ASC-CM from each day (6, 7 or 8) for each replicate (n=4) was combined to create a “batch” mixture to achieve adequate volumes for all experiments and eliminate any potential variability between ASC-CM from different days.

2.9. ASC-CM Stratification into Molecular Weight Fractions

Collected ASC-CM underwent a series of centrifugation filtrations via 100 kDa, 30 kDa, 10 kDa, and 3 kDa molecular weight cutoff filters, starting with 100 kDa, per manufacturer's instructions. Each centrifugation step was carried out at 4000 g for 30-45 minutes, the concentrate in the upper chamber was saved, and the filtrate in the lower chamber was collected and used for the next molecular weight filtration/centrifugation step until all that remained was a <3 kDa filtrate. This resulted in an ASC-CM concentrate (~50-100×) for each molecular weight range.

### 2.10. ASC-CM Protein Quantification

ASC-CM samples were quantified via total protein analysis with QuickDrop absorbance at 280 nm, BCA, and Bradford (Coomassie). Samples were then used for downstream analysis via protein quantification with a Pierce™ BCA Protein Assay Kit (Invitrogen; Cat. #23225), Pierce™ Coomassie “Bradford” Protein Assay Kit (Invitrogen; Cat. #23200), and a QuickDrop (Molecular Devices; SpectraMax QuickDrop Micro-Volume Spectrophotometer) quantification via absorbance at 280-nm. Relative protein content was determined and back-calculated to determine what the relative concentration was within media before molecular weight concentration steps (e.g., 10-mL of media concentrated to 500-μL was a 20× concentration). Relative protein content for each molecular weight fraction was determined relative to “Full” ASC-CM before stratification. Assays were performed with technical and biological replicates (n=4).

2.11. ASC-CME xtracellular Vesicle (EV) Production

EVs were isolated via ASC-CM centrifugation at 4000 g for 30-minutes through a 100 kDa centrifuge filter (same as above) followed by washing with PBS and re-centrifugation at 4000 g for 5-minutes through the 100 kDa filter, for a total of 3 washes. EVs were precipitated from the remaining “>100 kDa” concentrate overnight using a ExoQuick-TC kit (SBI; Cat. #EXOTC10A-1), per the manufacturers protocol. EV were resuspended in PBS and aliquots were removed and used to quantify protein content as an indirect measure of EV content via QuickDrop, BCA, and Bradford (Coomassie). Relative EV protein fraction was compared to total protein within “>100 kDa” and “Full” ASC-CM fractions. Additionally, purified EV samples were then evaluated via Nanoparticle Tracking Analysis (NTA; Malvern Panalytical; Nanosight LM10) to further determine both concentration and size distribution of particles extracted from the ASC-CM, with exosomes typically ranging from 25-250 nm. Unused (not exposed to cells) serum-free ASC media underwent the same processing and was used to establish/determine baseline EV/particulate levels, which were negligible. EV particle counts of serum-free control ASC media was negligible and did not warrant a full workup, thus only a comparative analysis between 2D and 3D was fully conducted.

### 2.12. KC Functional Activity After ASC-CM Treatment

ASC-CM was used as a “supplement” for the Keratinocyte Growth Media (KC-GM) and dosed at a 2:1 ratio based on initial volume of ASC-CM (i.e., 20 mL of ASC-CM was concentrated to 200 μl and added to 10 mL of KC-GM). KCs were plated in 2D and allowed to acclimate and achieved desired confluences (>24 hours). KC-GM was then removed, cells were washed, ASC-CM was applied for 24-hours, and experimental assays for metabolic, mitochondrial, proliferative, or migratory activity were then performed per the manufacturer's instructions. Metabolic activity was assessed via PrestoBlue (as previously described), proliferation was assessed via PicoGreen (as previously described), mitochondrial activity was assessed via MitoTracker (as previously described), and Migratory activity was assessed via a scratch “wound” assay. KC scratch assays were performed to evaluate changes in wound size/area as a surrogate measurement of KC migration after inflicted a scratch within a confluent monolayer of KCs (n=4). Migration images were taken using an ImageXpress Micro XLS Imaging System (Molecular Devices) and the percent of wound area closed at 24-hours was determined via ImageJ analysis).

### 2.13. KC Senescence

KC senescence was performed via immunofluorescent labeling of β-galactosidase activity with the CellEvent™ Senescence Green Detection Kit (Invitrogen; Cat. #C10850), per manufacturer's instructions. KCs from P2 or P3 were utilized for this study. KC-GM was removed from KC culture and ASC-CM stratified concentrates were dosed as a supplement to KC-GM for 24-hours. KCs within KC-GM were used as a baseline measurement of senescence. After 24-hours, KCs were fixed and stained. Hoechst 33342 was used as a counterstain to identify nuclei. Senescence characterization was carried out in quadruplicate (n=4), with five (5) field of view images taken per biological replicate for a total of twenty (20) measurements per sample.

### 2.14. KC Protein Expression

Immunolabeling of cells was utilized for both ASCs and KCs. In brief, cells were washed 3× with HBSS, fixed with 4% PFA, and washed again 3× with HBSS. Cells were incubated in blocking buffer, which consisted of 2% donkey-serum with/without 0.1% Triton-X in HBSS, for at least 1-hour. Cells were then incubated with primary antibodies at 4° C. overnight. All primary antibodies were obtained from Abcam unless otherwise stated and included vimentin (ab8978), keratin 16 (ab76416), keratin 10 (ab76318), keratin 5 (ab52635). The next day cells were washed 3× with blocking buffer followed by application of secondary antibodies for 1-hour, then 3× washes with HBSS again. Cells were counterstained with an immunofluorescent nuclear marker, Hoechst 33342 and for some samples Alexa Fluor 488 Phalloidin. Secondary antibodies were derived in donkey and obtained from Invitrogen (n=3).

Western blotting was performed as described previously. Briefly, cell lysates were prepared using RIPA buffer with a protease inhibitor cocktail (Cat. #P8340). Protein concentrations were obtained via QuickDrop absorbance at 280 nm and were analyzed by SDS-PAGE via running protein on a NuPage 4-12% Bis-Tris gel (Invitrogen; Cat. #NP0321BOX), transferred onto PVDF membranes, and immunoblotted. Primary antibodies were all obtained from Abcam and included vimentin, CD9 (ab263019), CD63 (ab134045), CD81 (ab109201), Tsg101 (ab125011), GAPDH (ab8245), β-tubulin (ab6046), keratin 16, keratin 10, keratin 5. Secondary antibodies were derived in donkey and obtained from Invitrogen. Blocking buffer consisted of 2% donkey-serum in HBSS.

### 2.15. KC Gene Expression

RNA was isolated and purified as described in above, in brief an RNeasy Mini Kit (Qiagen) was used according to manufacturer's instructions. Untreated KCs cultured in KC-GM served as a control for KC analyses. Purity of cDNA samples was assessed with a QuickDrop spectrophotometer (Molecular Devices), with a 260/280 absorbance ratio>1.8 was designated as pure. Individual qPCR primers (Qiagen; Cat. #330001) were purchased to perform RT-qPCR on CDKN2A, CTNNB1, CDH1, CDH2, FLG, K10, K16, K5, TWIST1, VIM, and CCNDL. GAPDH was used as an endogenous control for these samples as well. The entire list of GeneGlobe IDs is listed in Table 2.

TABLE 2

| Gene | 2D Fold Change | 3D Fold Change | Gene | 2D Fold Change | 3D Fold Change | Gene | 2D Fold Change | 3D Fold Change |
|---|---|---|---|---|---|---|---|---|
| A [symbol] | [symbol] | [symbol] | [symbol] | [symbol] | [symbol] | [symbol] | [symbol] | [symbol] |
| [symbol] A [symbol] | [symbol] | [symbol] | [symbol] | [symbol] | [symbol] | [symbol] | [symbol] | [symbol] |
| [symbol] ALCAM | [symbol] | [symbol] | [symbol] | [symbol] | [symbol] | [symbol] | [symbol] | [symbol] |
| [symbol] A [symbol] | [symbol] | [symbol] | [symbol] | [symbol] | [symbol] | [symbol] | [symbol] | [symbol] |
| [symbol] A [symbol] | [symbol] | [symbol] | [symbol] | [symbol] | [symbol] | [symbol] | [symbol] | [symbol] |
| [symbol] B [symbol] F | [symbol] | [symbol] | [symbol] | [symbol] | [symbol] | [symbol] | [symbol] | [symbol] |
| [symbol] B [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] C [symbol] | [symbol] | [symbol] | [symbol] | [symbol] | [symbol] | [symbol] | [symbol] | [symbol] |
| [symbol] C [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] ENG (CD [symbol] | [symbol] | [symbol] | [symbol] | [symbol] | [symbol] | [symbol] | [symbol] | [symbol] |
| E [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] G [symbol] | [symbol] | [symbol] | [symbol] | [symbol] | [symbol] | [symbol] | [symbol] | [symbol] |
| G [symbol] | [symbol] | [symbol] | [symbol] | [symbol] | [symbol] | [symbol] | [symbol] | [symbol] |
| G [symbol] | [symbol] | [symbol] | [symbol] | [symbol] | [symbol] | [symbol] | [symbol] | [symbol] |

*Denotes Significant Different Between 2D and 3D with a p > 0.05 [symbol]
indicates data missing or illegible when filed

### 2.16. EV In Vitro Tracking

EVs previously isolated from the ASC-CM were fluorescently labeled with the lipophilic membrane stain DiI (Invitrogen; Cat. #D282) at a concentration of 1-μM. The labeled EVs were washed with PBS and re-centrifuged at 3500 g for 15-minutes through the 100 kDa filter, for a total of 3 washes and centrifugations to remove excess dye. DiI-labeled EVs were added to KC-GM, which was then applied to KCs for 24-hours. Media was removed and KCs were washed 2×, fixed, counterstained with Hoechst 33342, and Alexa Fluor 488 Phalloidin and observed under a fluorescence microscope.

### 2.17. Dosing of EVs for 2D Versus 3D Quality Comparison

EVs isolated from 2D and 3D were then resuspended at known concentrations and added to KC-GM for a final concentration of 5, 25, or 250 μg/mL of EV protein within the KC-GM. EV concentration calculated based on average of all three protein analyses (QuickDrop, BCA, Bradford)(n=4).

### 2.18. Imaging Analysis

Processing and analysis of images was performed with ImageJ and CellProfiler. All sets of images and image analyses were automated with a standardized pipeline and treated the same way across all similar image sets.

### 2.19. Statistical Analysis

All data were reported as means with standard error of mean (s.e.m.). Characterization analyses of ASC populations for metabolic, functional, and phenotypic data were evaluated with a One-way ANOVA. All Secretome stratification data were analyzed with a Two-way ANOVA. Characterization of extracellular vesicles (exosomes) was assessed via an unpaired student's t-test. Exosome dosing studies were also evaluated with a Two-way ANOVA. A minimum of four replicates (n=4) was used unless otherwise stated. Data was tested for normality via Shapiro-Wilk and Kolmogorov-Smirnov tests and plotted with a QQ plot. GraphPad Prism 9.4.2 software (La Jolla, CA) was used for the analyses and a p<0.05 was considered significant. CellProfiler™ and ImageJ were utilized for image processing.

### 3. Results

### 3.1. Evaluating the Role of Mesenchymal Stem Cells in Wound Healing

### 3.1.1. Characterization of ASC Populations

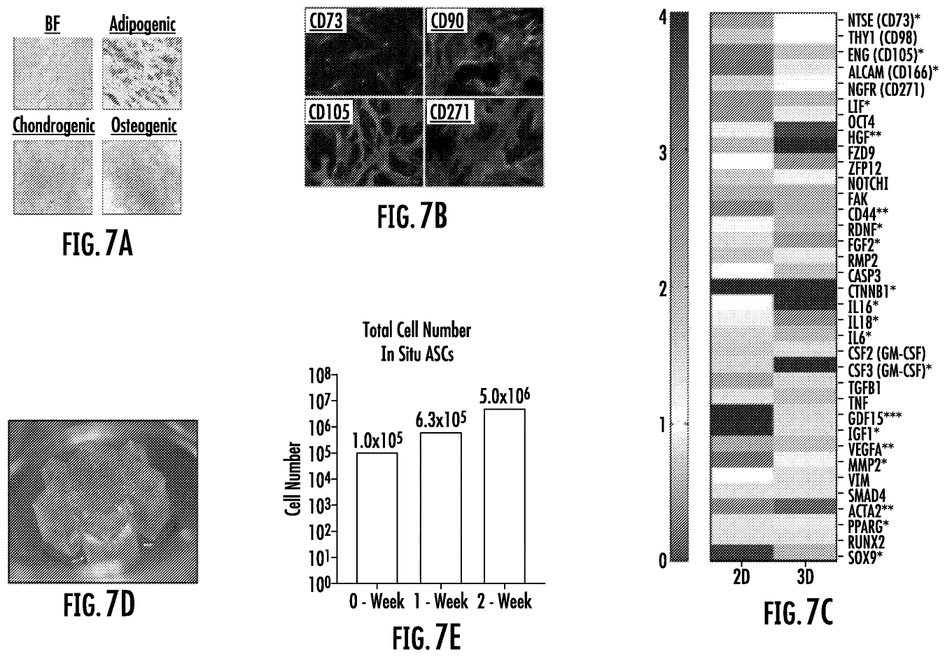

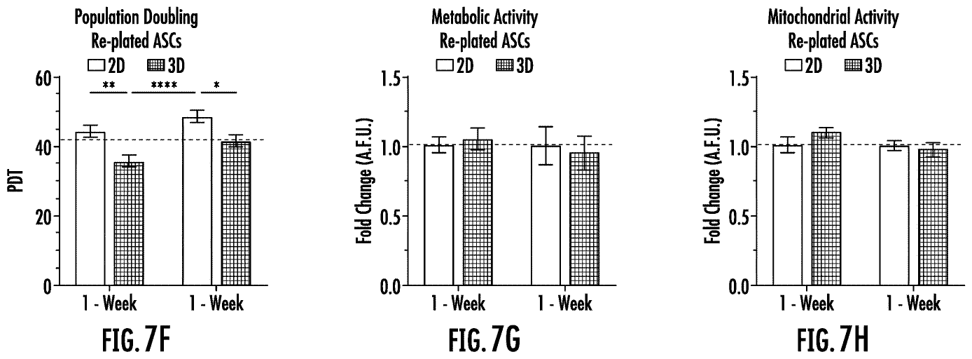

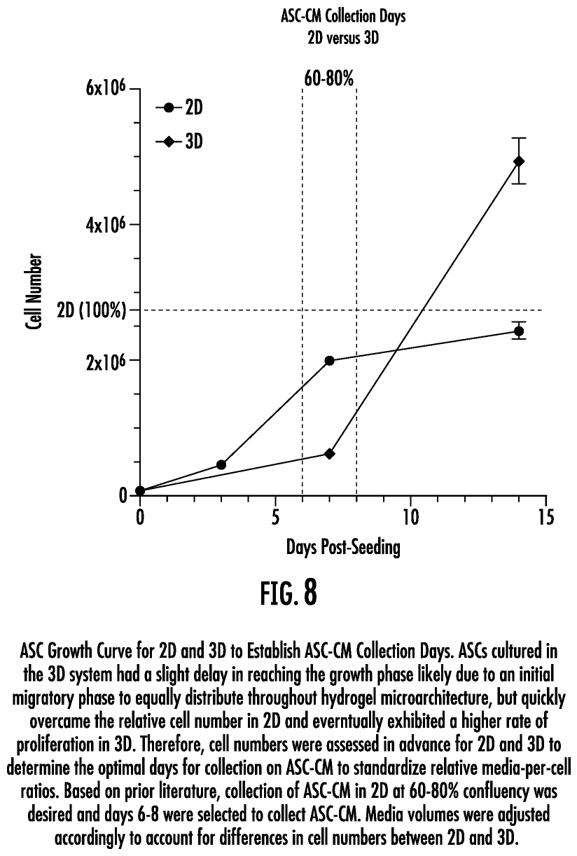

According to the International Society for Cellular Therapy, the minimum criteria for a cell population to be considered a MSC is they are adherent (FIG. 7A), exhibit trilineage multipotent potential (FIG. 7A), and express MSC “stem-like” surface markers (FIG. 7B), including positive staining for CD73/90/D105. Similarly, ASCs were then cultured in 2D or 3D for one (1) week and assessed for MSC phenotypic gene expression relative to the initial ASC population (FIG. 7C). ASCs within the 3D culture (FIG. 7D) exhibited a higher retainment of key MSC and MSC-like markers, whereas cells in 2D demonstrated a significant decline (FIG. 7C). Additionally, an in situ population doubling experiment was performed to assess relative cells numbers in 2D and 3D to determine optimal ASC-CM collection for future experiments (FIG. 7E). ASC-CM was collected when 2D ASCs exhibited 60-80% confluency, based on prior literature, and determined to be on days 6-8 (FIG. 8).

ASCs in our tissue-mimetic 3D hydrogel system (e.g., TMHB system) were isolated and re-plated into 2D and further assessed for functional alteration in proliferation, metabolic, or mitochondrial activity, relative to traditional 2D culture. ASCs in 3D retained a lower population doubling time (PDT) upon re-plating in 2D, indicating cells were able to proliferate more rapidly (FIG. 7F). Moreover, the metabolic (FIG. 7G) and mitochondrial (FIG. 7H) health and activity of re-plated ASCs demonstrated no significant differences in 2D or 3D, over the time course of two weeks in either system.

### 3.2. Molecular Weight Stratification of Secretome Differentiates Wound Healing Involvement

### 3.2.1. Larger MW Secretome Fraction is Key Driver of KC Wound Healing Activity

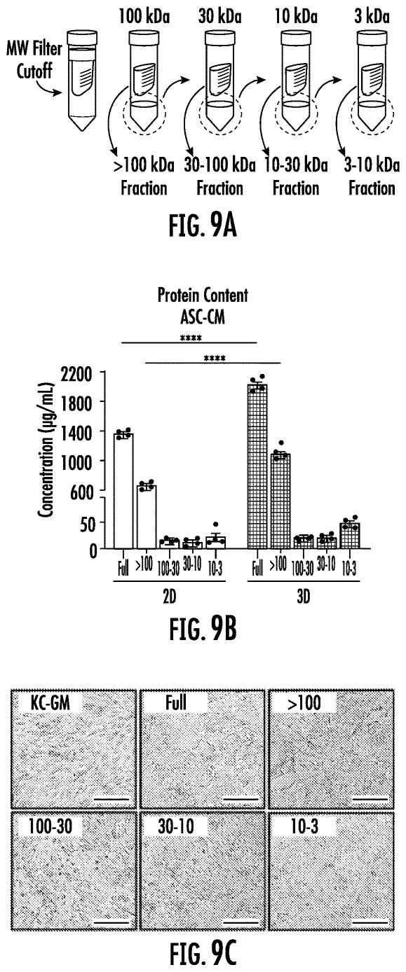

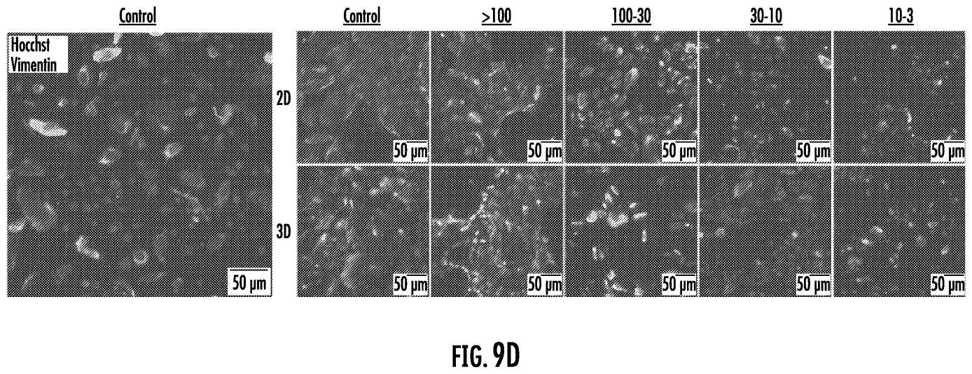

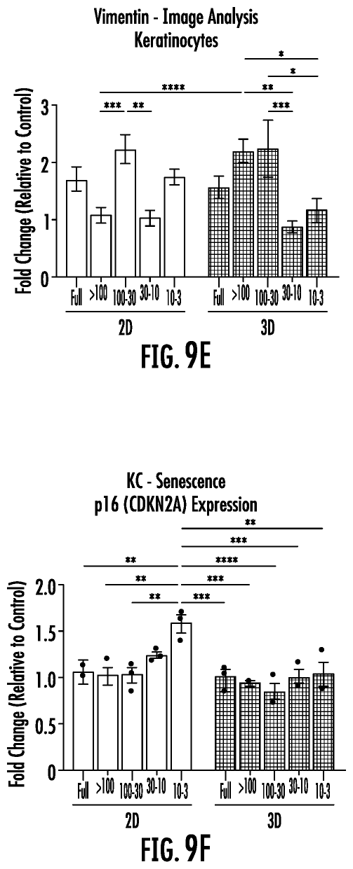

ASC-CM collected from 2D and 3D was stratified by molecular weight (FIG. 9A) and each fraction was analyzed for total protein content (FIG. 9B) and ability to modulate a KC functional activity. ASCs within the tissue-mimetic system secreted ~40-50% more total protein than their 2D counterparts, where the “Full” and “100 kDa” fractions were significantly greater in 3D relative to 2D (FIG. 9B). When accounting for total protein, the “100 kDa” fractions made up about −49% and ~53%, for 2D and 3D, respectively. All other tested fractions maintained a similar relative protein content between 2D and 3D.

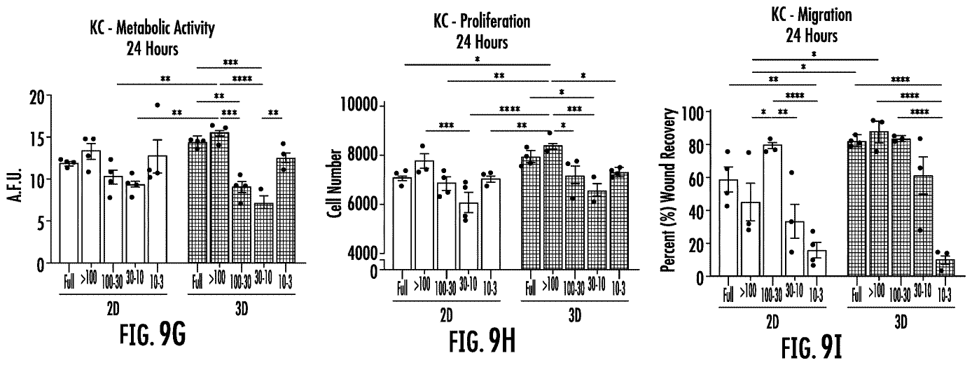

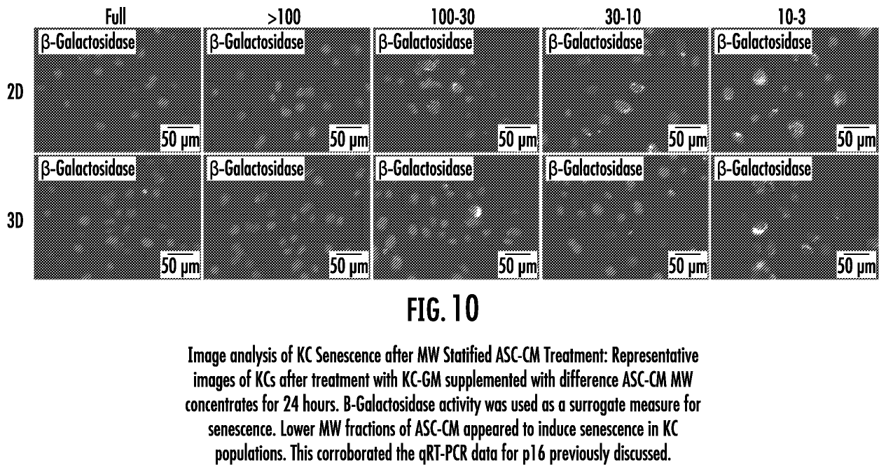

Previous studies have demonstrated that the ASC secretome can modulate the morphology of KCs to become more spindle-shaped and stratified, potentially indicating a potential change in KC phenotype that may be important and related to the wound healing capacity of ASC-CM. Control KCs (cultured with KC-GM) maintained a more homogenous rounded morphology, whereas the “100 kDa” fraction of ASC-CM promoted a morphological switch in KCs from both 2D and 3D culture towards a stratified cell sheet with spindle-like cells (FIG. 9C). KCs treated with lower MW fractions of ASC-CM appeared to exhibit smaller cell sizes and a rounded morphology, without cell sheets. Interestingly, KCs treated with higher MW fractions of ASC-CM, “100 kDa” and “100-30 kDa” fractions, demonstrated a significant increase in expression of the mesenchymal and migratory marker Vimentin (FIG. 9D-E). Conversely, KCs treated with lower MW fractions, from either 2D or 3D, exhibited increased expression of the senescence-related markers p16 and β-Galactosidase (FIGS. 9F and 10).

The metabolic, proliferative, and migratory activity of KCs were utilized are surrogate measures of in vitro wound healing activity. The “Full” ASC-CM from this 3D system has demonstrated benefits, relative to 2D, in prior studies. The “100 kDa” ASC-CM fraction for both 2D and 3D demonstrated a slight increasing trend, relative to “Full”, in its ability to enhance KC metabolic (FIG. 9G) and proliferative (FIG. 9H). Whereas only the 3D group of “100 kDa” appeared to enhance migratory activity of KCs (FIG. 9I), with the 2D “100 kDa” group exhibiting a slight decrease, relative to “Full”. Additionally, the “100-30 kDa” fraction appeared to drive KC migratory activity for both 2D and 3D (FIG. 9I). The relative activity of the lower MW fractions of ASC-CM have similar capabilities toward augmenting metabolic and proliferative activity in KCs, when comparing the 2D or 3D groups to their counterparts. However, lower MW fractions demonstrate less capacity to augment KC functional activity than the larger MW fractions overall (i.e., “100 kDa and “Full”).

### 3.3. ASC-EVs Drive Key Regenerative Wound Healing Pathways in Keratinocytes

3.3.1. ASC Populations within Tissue-Mimetic System Favor Secretion of EVs

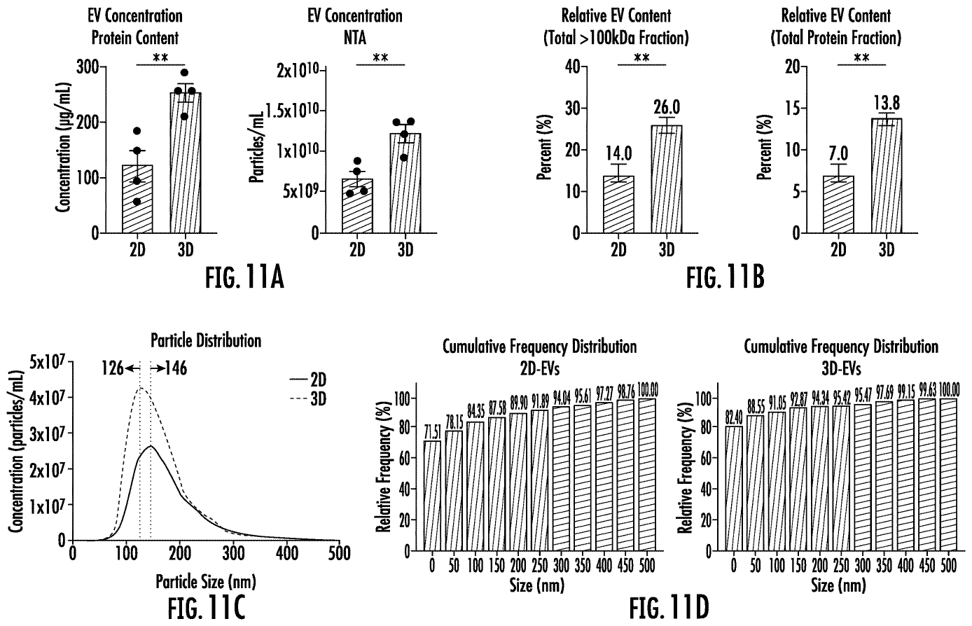

Production and characterization of EV quantity and size distribution within ASC-CM was evaluated between 2D and 3D culture systems. Total EV production was increased over 2-fold in 3D relative to 2D (FIG. 11A). Similarly, the relative secretion/composition of EV-to-Protein within the secretome was approximately 2:1, with ~14% (3D) or 7% (2D) of secreted protein being due to EVs (FIG. 11B). EVs were then characterized via exosome markers and nanoparticle tracking analysis (NTA) to determine whether exosomes were the main EV type being produced (e.g., determine size distribution of the EVs—FIG. 11C. Additionally, NTA further validated an ~2-fold increase in particles with 3D ASC-CM (FIG. 11A), in addition greater than 90% of the particles exhibiting a size distribution within the range of exosomes (25-250 nm; FIG. 11C and D). Notably, particles from 3D ASC-CM exhibited a trend towards a slightly smaller average size than 2D, but not significant.

3.3.2. ASC-EVs within Tissue-Mimetic System Contain More Potent Re-epithelialization Stimulus

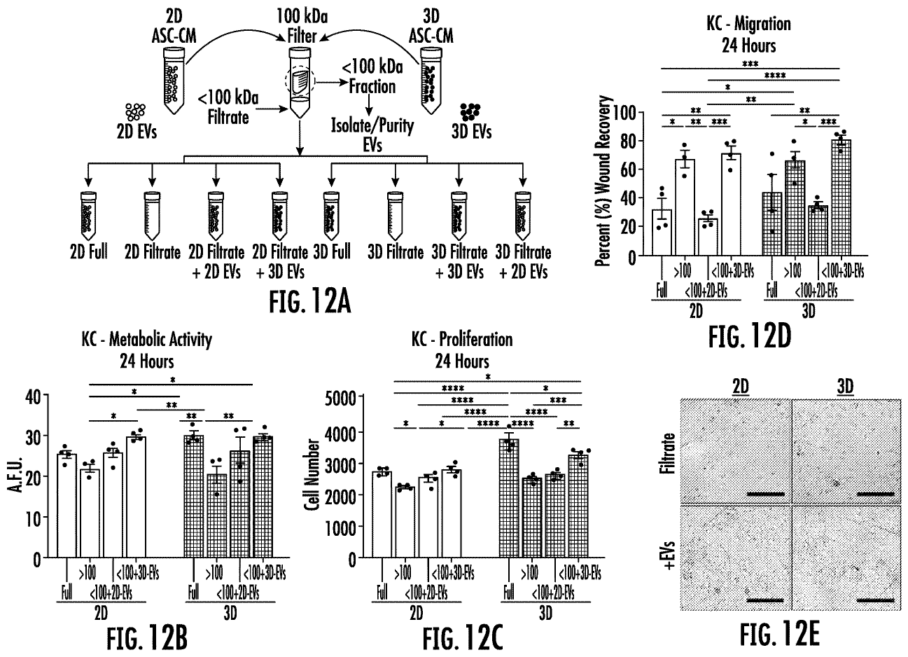

To isolate and observe the role of ASC-derived EV/exosomes in modulating KC re-epithelialization activity, ASC-CM was separated into >100 kDa (EV containing) and <100 kDa (Filtrate) fractions. Subsequently, EV/exosomes were extracted from the >100 kDa fraction to separate them from soluble protein within the >100 kDa fraction and used to dose 2D and 3D ASC-CM “Filtrate” fractions (FIG. 12A). Both 2D and 3D ASC-CM “Filtrate” had similar effects on KC metabolic (FIG. 12B), proliferative (FIG. 12C), and migratory (FIG. 12D) activity. Addition of 2D-EVs resulted in a slight increase in metabolic and proliferative activity of KCs, but 3D-EVs was the only group to result in a significant increase (FIG. 12B-C). Notably, the migratory activity of KCs was significantly enhanced when ASC-CM filtrate was treated with 3D-EVs, whereas 2D-EVs significantly decreased the migratory capacity of ASC-CM filtrate (FIG. 12D).

When comparing the morphology of KCs treated with either “Filtrate” or “Filtrate+EVs”, KCs demonstrated morphological changes seen previously. KCs treated with ASC-CM “Filtrate” had minimal morphological altercations, but addition of ASC-EVs from 2D or 3D resulted in a similar morphology to KCs seen previously after treatment with “Full” or “100 kDa” fractions (FIG. 12E).

### 3.4. EV Dosing Assessment Reveals Several KC Wound Healing Responses are Dose-Dependent

### 3.4.1. 3D-EVs Enhance Expression of Basal and Suprabasal Cytokeratins in a Dose-Dependent Manner

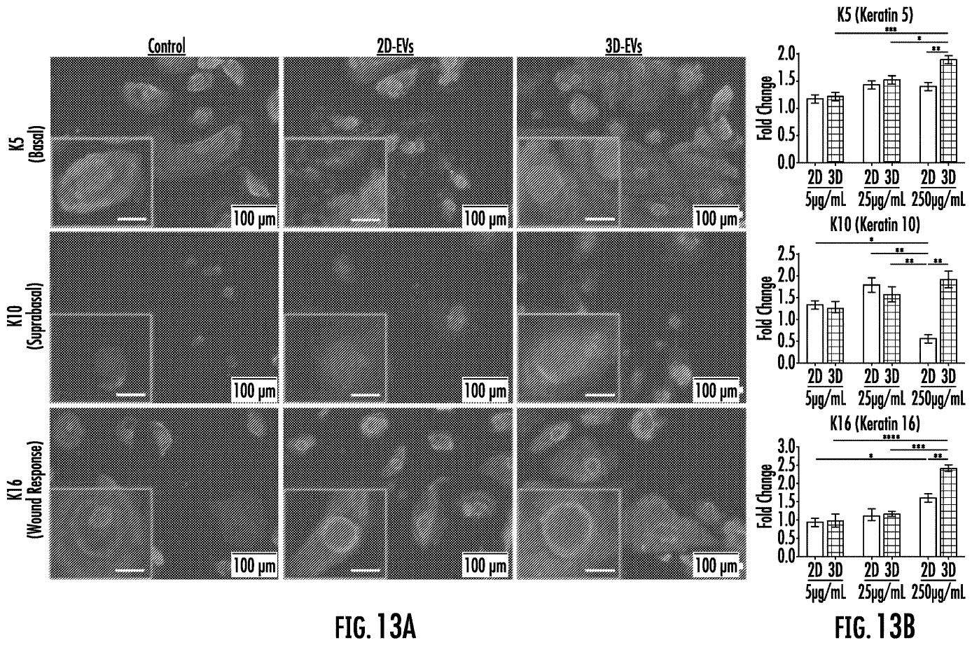

An EV dosing at 5-, 25-, and 250-μg/mL was performed to further understand ASC-EV functionality/quality and evaluate whether KC phenotype responded in any dose-dependent manner via immunolabeling (FIG. 13A), and qRT-PCR (FIG. 13B). Immunolabeling and western blotting indicated that KCs treated with ASC-EVs exhibited varying degrees of protein for K5 (basal layer), K10 (suprabasal layer), and K16 (wound healing responsive) cytokeratins. Only K16 exhibited increased expression levels when treated with the highest dose (250-μg/mL) of 2D-EVs, with K10 significantly decreasing after 2D-EV treatment. Treatment with the highest dose of 3D-EVs demonstrated the capacity to significantly increase expression of all three cytokeratins at a protein and RNA level (FIG. 13A-B). Additionally, only K16 exhibited a dose-dependent response to 2D-EVs, whereas K5, K10, and K16 all exhibited a dose-response to 3D-EVs (FIG. 13B).

### 3.4.2. EMT and Epidermal Regeneration of KCs Exhibit Dose-Dependent Response to ASC-EVs

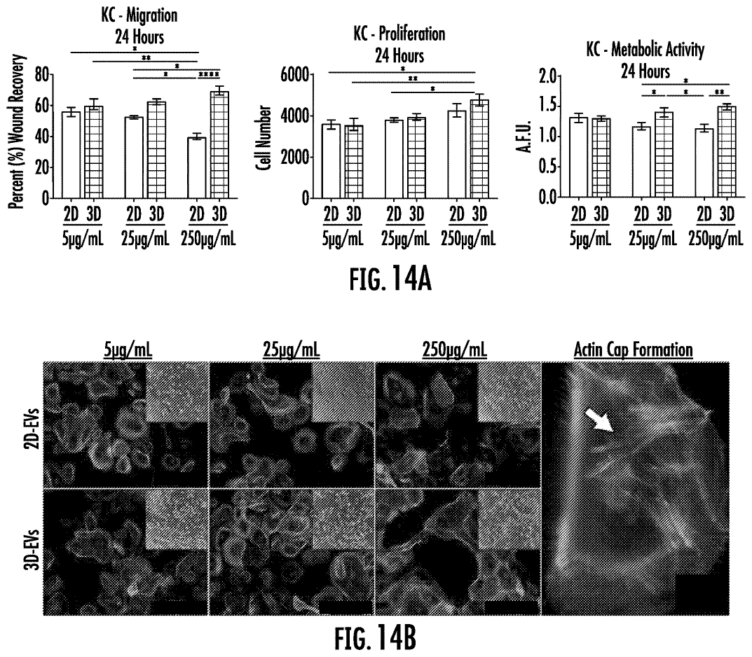

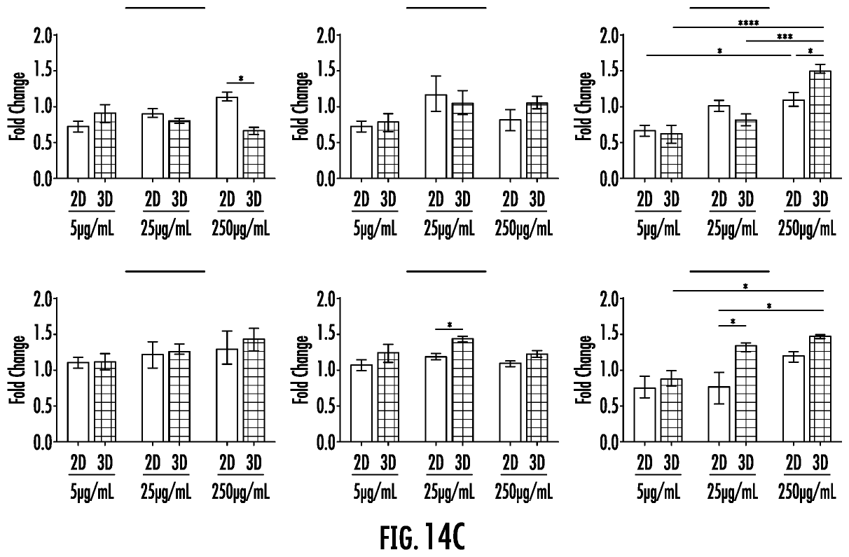

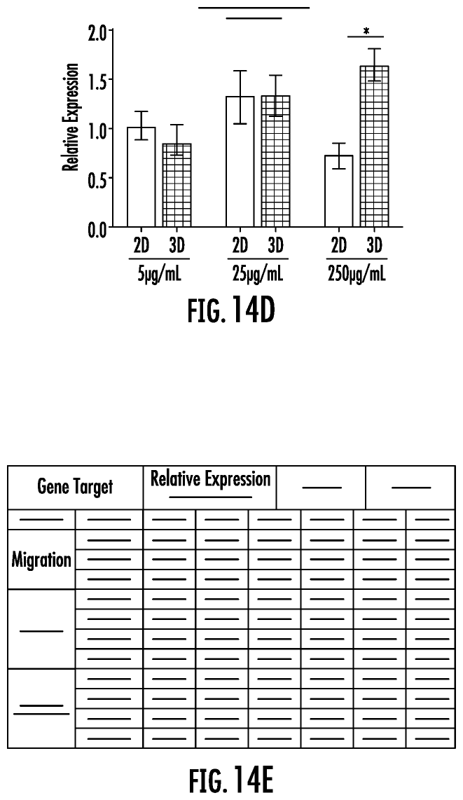

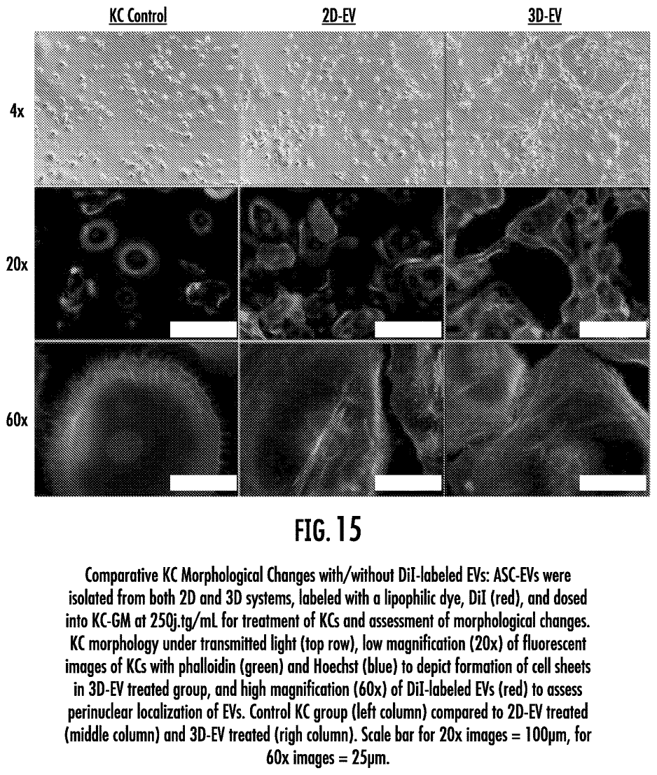

KC metabolic and migratory activity exhibited a decreasing trend when treated with 2D-EVs (R²=0.42 and 0.98, respectively) but an increasing trend when treated with 3D-EVs (R²=0.76 and 0.96, respectively); whereas KC proliferative activity exhibited a positive dose response for both 2D-EVs (R²=0.97) and 3D-EVs (R²=0.96) (FIG. 14A). Notably, KC morphological changes exhibited a dose-dependent response. With increasing EV dosing, KCs exhibited increased spindle-like transformations, cell clustering, cell sheet formation, and cytoskeletal modulation with actin-cap formation (FIGS. 14B and 15). Notably, morphological changes were observed in both 2D-EVs and 3D-EVs, but more pronounced with 3D-EVs treatment.

Additionally, a number of key gene markers for KC wound healing activity and phenotype were assessed to determine whether KC gene expression exhibited similar changes as the functional and proteomic evaluations (FIG. 14C). The expression of key junctional/migratory markers CDH1 (E-Cadherin) and CDH2 (N-Cadherin) did not demonstrate any significant dose-dependent response to ASC-EVs, although the highest dose of 3D-EVs did result in a significant decrease in expression of CDH1 (relative to 2D-EVs). Whereas the expression of the proliferative marker CCND1 (Cyclin D1) did demonstrate a dose response with increasing ASC-EVs concentration from both 2D and 3D (FIG. 14C). Similarly, expression of FLG (Filaggrin), a marker for suprabasal differentiation (granular layer), and VIM (Vimentin), a marker for KC migration, both exhibited a dose response to ASC-EVs, with 3D-EVs at 250-μg/mL providing a significant increase relative to 2D-EVs. Whereas VIM (Vimentin), a marker for KC migration, did not exhibit a dose response (FIG. 14C). TWIST1, a marker for epithelialization and an EMT-like response, exhibited a dose response to ASC-EVs with a more potent effect from 3D-EVs (FIG. 14C). When considering the relative expression of both CDH1 and CDH2, KCs exhibited a significantly increasing trend towards favoring expression of CDH2 (N-Cadherin) (FIG. 14D). The relative correlation of each gene expression marker is denoted in FIG. 14E.

### 4. Discussion

The diverse array of tissue types and cell populations involved in wound healing, in addition to the wide-ranging patient populations and various types of wounds, highlights the need for therapies that are tailored for specific applications. Recently, investigations into the utilization of the dynamic secretome of MSCs has demonstrated the potential for a unique opportunity for advancing the field of regenerative medicine and personalized wound healing modalities. To date, the secretome of MSC-like populations have been shown to stimulate a variety of wound healing functions, including modulation of fibrotic activity in fibroblasts, promotion of angiogenic activity in endothelial cells, and increased keratinocyte migratory activity.

Although preliminary data with MSC biologics is promising, in order to tailor regenerative biologic therapies more effectively there is need to differentiate the role/activity of MSC secretome components and generate modalities to reproducibly create efficacious products. Previous studies have begun investigating the utility of a number of MSC secretome elements, including looking at the effect of a single component, such as production of a specific growth factor (e.g., VEGF) or exosomes, whereas other studies have focused on the effect of bulk (i.e., Full) conditioned media from MSCs. Ultimately, the ideal composition of factors will be dependent on the application, though the diverse milieu of factors secreted by MSCs offers a dynamic balance of factors that can be applied to a variety of clinical scenarios.

However, to date, most research into MSC biologics utilize 2D culture systems, and the ones that do use 3D systems focus on using systems, such as hydrogels, for cell delivery and/or production of a specific secretory product via a “priming stimulus” (e.g., fibrin hydrogels enhance VEGF secretion). Thus, current data is incomplete and obstructed due to the detrimental effects of traditional 2D systems on MSC phenotype, viability, and regenerative capabilities. The unphysiological environment of 2D systems drastically hinders the standardization and reproducibility of MSC-based therapies. Similarly, many current 3D systems (e.g., spheroids and microcarriers), although more efficacious than 2D for a variety of applications, still lack tissue-mimetic properties and/or have diffusional constraints that result in cellular heterogeneity and hinder large-scale expansion efficiency that would be necessary for future clinical therapies.

Therefore, for this working example we utilized a tissue-mimetic 3D hydrogel system (e.g., TMHB system) that we have previously shown to enhance the retainment of non-senescent, MSC-like populations, relative to standard 2D culture. When designing a tissue-mimetic system, there are a number of important viscoelastic properties of tissue to consider, including the compressive, storage, and loss moduli, which are denoted in Table 1 for our hydrogel system as it compares to previously published data on adipose tissue.

Two key benefits of this system are that it allows the easy collection of secreted byproducts due to unique architectural design. Unlike previously investigated poured/molded 3D hydrogels in literature that lack defined microchannels and structure, the micro- and macro-architecture provides a unique opportunity for continuous expansion of cells within a hydrogel system while permitting the easy and continuous collection of secreted byproducts, such as EVs. The microchannels help circumvent diffusional limitations that are associated with traditional hydrogel systems, subsequently increasing the efficiency of secreted biologics collection. Additionally, the tissue-mimetic environment results in MSC-like populations that retain their adaptive and regenerative capabilities, allowing them to respond to stimuli more appropriately, like they would in vivo. To support and highlight the relative benefit of the 3D system on MSC phenotype, an RNA array was performed and demonstrated that ASCs exhibited a significant retainment of key MSC and MSC-like markers in 3D, relative to 2D. The improved ASC phenotype within this adipose-like microenvironment results in a secretome composition that enhances regenerative responses, relative to 2D. In part, this is likely due to a combination of increased secretion of anti-regenerative factors from 2D cultured cells, such as ROS and senescence-promoting factors (FIG. 9F), in addition to the increased secretion of pro-regenerative factors from 3D cells.

The ability to achieve targeted and rapid degradation of the hydrogel substrate allowed easy extraction of ASCs for RNA analysis. To support and highlight the relative benefit of our 3D system on MSC phenotype, an RNA array was performed and demonstrated that ASCs exhibited a significant retainment of key MSC and MSC-like markers in 3D, relative to 2D (FIG. 1C and Table S1). As we can see, surface markers associated with an MSC-like phenotype, including NT5E (CD73), THY1 (CD90), ENG (CD105), ALCAM (CD166), and NGFR (CD271), retain a gene expression profile similar to the primary (P1) ASC populations within the 3D hydrogel, whereas the 2D system resulted in a precipitous decline in their expression. Moreover, ASCs in 3D exhibited retained or increased expression of several “stemness” associated markers, including LIF, OCT4, HGF, ZFP42, and NOTCH1, in addition to increased retainment of multipotency markers PPARG (adipogenic), RUNX2 (osteogenic), and SOX9 (chondrogenic). Notably, there is a significant decrease in CASP3 expression in 3D-ASCs, an important marker for cell health and induction of cell death via apoptosis.

Even though the same seeding density was utilized initially, variations in cellular proliferation were expected, which can be seen when looking at the relative proliferative activity via the population doubling time (PDT) of ASCs extracted from 2D and 3D culture systems (FIG. 7F). Therefore, cell numbers were assessed in advance for 2D and 3D to determine the optimal days for collection of ASC-CM to standardize relative media-per-cell ratios. Additionally, both 2D and 3D ASCS were at P2 upon seeding and remained in culture for 8 days for this study. Thus, any differences noted are a reflection of the culture environment and not subculturing effects. Based on prior literature, collection of ASC-CM in 2D at 60-80% confluency was desired.

Previous studies have demonstrated a positive impact of the MSC secretome on KC wound healing activity. However, to date, the data is limited and there are mixed results as too what components may be driving specific activities, such as migration and proliferation, due to the adaptive and dynamic nature of the secretome. Therefore, KC functional and phenotypic assessments were performed as an indirect surrogate measure and qualification of MSC secretome components from either a 2D or 3D culture system. Molecular weight stratification was utilized due to the minimally invasive nature of this methodology that does not require solvents or other factors to precipitate and/or isolate proteins from conditioned media, in addition to the ability to compartmentalize compounds into predefined fractions and standardize a consistent processing methodology. Notably, ASCs cultured within our 3D culture system exhibited enhanced secretion of proteinaceous compounds, relative to 2D (FIG. 9B). Upon stratification, only the “100 kDa” fraction was significantly increased in 3D, relative to the 2D counterparts. Suggesting that the enhanced protein production in 3D is a result of the “100 kDa” fraction, which consists primarily of larger growth factors/cytokines, matrix-related proteins, and EVs.

KC functional activity was then evaluated to assess ASC-CM secretome activity for each MW fraction. The functional benefits of ASC-CM from the “100 kDa” fraction demonstrated the greatest positive impact on KC activity and paralleled the effect and benefits seen in the “Full” media group, including KC morphological, metabolic, proliferative, and migratory activity. Whereas the lower MW fractions had less effect on KC functional activity. A closer look demonstrated that the effect of the lower MW fraction from both 2D and 3D are similar but there is a significant drop-off in effect from the 3D “100 kDa” fraction (FIG. 9G-I). This is potentially due to the increased benefits from the 3D “100 kDa” fraction, rather than a negative effect of lower MW 3D compounds.

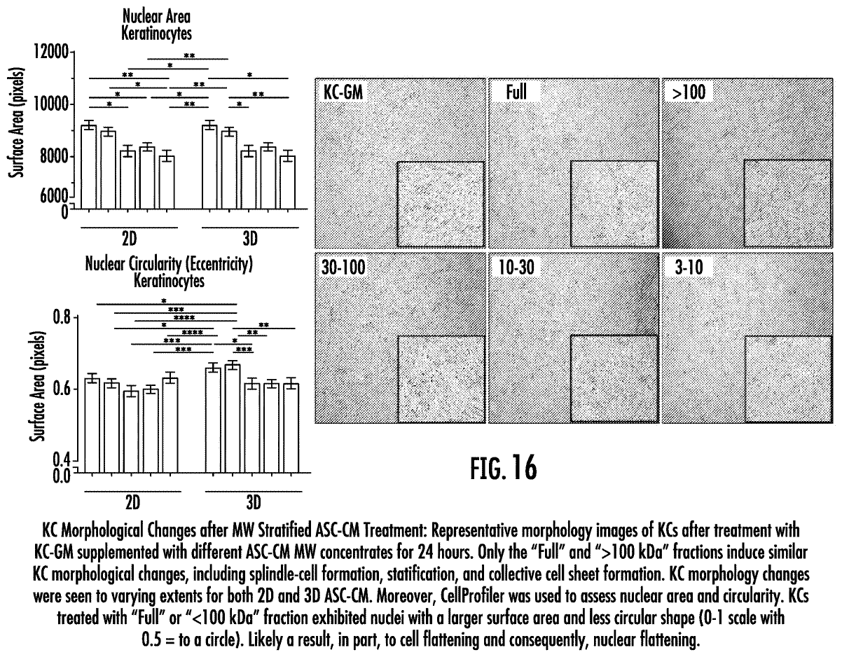

Interestingly, both 2D and 3D “Full” and “100 kDa” groups induced significant morphological changes in KC populations that resembled more flattened, stratified, and spindle-shaped cells forming cellular sheets. Whereas lower MW fractions had minimal effects on the KC morphology. Moreover, further evaluation of KC nuclear size indicated an increase in nuclear surface area in the “Full” and “100 kDa” groups only (FIG. 16), potentially due to flattening of the cells seen in the morphological changes, resulting in a decrease in the cellular z-axis. Future studies will investigate the relationship between KC morphological changes and wound healing activity; however, some KCs are thought to undergo a form of an epithelial-to-mesenchymal transition (EMT) and suprabasalar differentiation during re-epithelialization, which could account for the morphological changes seen.

Previous studies have shown that senescent cell populations, including MSCs, can impair the regenerative activity of surrounding cell populations in a number of ways, including the secretion of factors that induce senescence. Notably, we have previously shown that ASCs cultured within this tissue-mimetic system delays the induction of ASC senescence, whereas 2D culture led to a rapid induction of senescence. Therefore, 2D cultured ASCs may be secreting protein or non-protein factors associated with senescence and decreased cell health that are then inducing senescence in KCs (FIGS. 7F and 10). Analysis of KCs after ASC-CM treatment indicated that the “10-3 kDa” MW fraction from 2D did have a greater propensity for inducing senescence in KC populations, with the 2D “30-10 kDa” fraction also demonstrating an increasing trend. Interestingly, the analogous fraction in 3D did not induce senescence. Since the relative amount of protein secreted in the lower MW fractions was similar between 2D and 3D, the senescence induction is not a result of protein quantity but rather due to either the protein quality/composition or non-proteinaceous factors, such as reactive oxygen species (ROS). Notably, impaired balance of a number of secretory factors is considered to be able to promote the progression of cellular senescence in a paracrine manner. Moreover, the senescence-associated secretory phenotype (SASP) of MSC-like populations has previously been shown to induce senescence in other cells, potentially via ROS or inflammatory factors within the 10-30 kDa range. However, the dynamic nature of the SASP warrants further investigations into the exact mechanisms of senescence induction.

To further investigate the mesenchymal-like morphology change and enhanced migratory activity of KCs treated with high MW ASC-CM fractions, Vimentin was assessed and found to be correlated to KC migratory activity. Vimentin was significantly increased in KCs treated with the “100 kDa” and “100-30 kDa” fractions in 3D, relative to the lower MW fractions, especially at the outer edges of cell sheets. However, only the “100-30 kDa” fraction was enhanced in 2D, with the “100 kDa” fraction in 2D demonstrating a reduction in capacity to promote KC migration and express Vimentin. This indicated that components within the “100 kDa” fraction are likely promoting KC migratory activity within the 3D ASC-CM and secretion of these factors is decreased/countered within 2D culture. Moreover, there is likely a secondary set of factors within the “100-30 kDa” fraction that is also promoting migratory activity of KCs that is similar between 2D and 3D. This data suggests that the “100 kDa” and “100-30 kDa” fractions of the ASC secretome contain compounds that modulate KC migratory activity. Thus, culture of ASCs within a tissue-mimetic system either enhances the secretion of positive “100 kDa” factors or 2D culture promotes the secretion of negative “100 kDa” factors, but does not alter the migratory factors within the “100-30 kDa” range. This is the first demonstration of the diverging effects of secretome fractions separated via MW and provides insight into the functional implications of specific biomodulatory fractions. For example, a potential “inversion” of a pro-regenerative migratory stimulus within the “100 kDa” range that is dependent on the culture system utilized. Additionally, previous studies have demonstrated that increased expression of vimentin at the leading migratory edge of the epidermis is a critical step to epithelial regeneration natively; while our data depicts that treatment with the “100 kDa” fraction of the ASC secretome can promote this physiological KC wound healing response.

Based on the observed benefits of the “100 kDa” fraction, further stratification of the “100 kDa” components was carried out to investigate whether EVs within the “100 kDa” fraction were driving the observed functional changes in KCs. Culture of ASCs within the tissue-mimetic system resulted in increased production of EV particles, including modulation of the relative proportion of EV-to-Protein (secreted). Indicating that the secretome dynamics of ASCs tend to favor secretion of EV particles rather than soluble protein when in a more tissue-mimetic (i.e., native) environment, with a >100% increase in EV-to-Protein secretion in 3D relative to 2D. Moreover, the relative size distribution of the EVs from ASCs in 3D trended towards a slightly smaller size, potentially suggesting that EVs are more likely exosomal in nature (25-250 nm) rather than MVs (100-1000 nm) or apoptotic bodies (200-2000 nm). This was further supported by the presence of the exosomal proteins CD9, CD63, CD81 and Tsg101. To the authors' knowledge, this is the first demonstration of a shift in MSC secretory dynamics towards favoring the secretion of EV/exosomes from an MSC-like population within a tissue-mimetic system rather than soluble protein (FIG. 12B), providing potential insight into native tissue signaling preferences. Whereas previous studies have only investigated total EV/exosome amount or effect in 3D systems without comparing to 2D or controlling for total relative secreted protein.

The role of the EV/exosomes from both 2D and 3D was then evaluated for ability to recover/improve KC functional wound healing activity by utilizing <100 kDa filtrate dosed with or without ASC-EVs to determine whether the benefits seen from the “Full” and “100 kDa” fractions are an effect of EVs. Since EVs are primarily located within the “100 kDa” fraction, the “<100 kDa Filtrate” groups plus ASC-EVs resemble “Full” media, from the stratification studies, but depleted of soluble “100 kDa” protein that is not derived from EVs. The previous MW stratification data suggested that the “100 kDa” fraction provided a slight bump in KC metabolic and proliferative activity, relative to the “Full” group, for both 2D and 3D ASC-CM. When separating out the EVs and dosing the “<100 kDa Filtrate”, we see that both 2D-EVs and 3D-EVs are able to reestablish the effects seen in the “Full” groups for KC metabolic activity. On the other hand, only dosing with 3D-EVs was able to significantly increase the effects of the “<100 kDa Filtrate” when it came to KC proliferative activity. Notably, KCs did exhibit a significant increase in proliferative activity in the “2D<100 kDa Filtrate+3D-EVs” group, suggesting that the contents within 2D-EVs may lack the same proliferative capacity as 3D-EV contents. Thus, the previous proliferative benefits seen in the “100 kDa” fraction for 2D may be non-EV derived or the EV stimulus is less robust.

Furthermore, comparing the effect of 2D-EVs to 3D-EVs on KC migratory activity reveals a significant differential. As we saw in the “100 kDa” fractions previously, there is an “inversion” of migratory signaling between 2D-EVs and 3D-EVs, where 3D-EVs enhance KC migration but 2D-EVs hinder KC migratory activity. These data support the stratification data that suggested that “100 kDa” fraction of 2D ASCs does not contain the same pro-migratory stimulus as their 3D counterpart, rather 2D-EVs may contain a possible anti-migratory stimulus for KCs. One possible explanation for this may be that the adaptive response of ASCs in 2D monolayers to the overcrowding and contact inhibition resulting in ASCs releasing anti-migratory (and possibly anti-proliferative) signals within EVs. Conversely, 3D culture results in more regenerative ASC populations and provides significantly more surface area to migrate which likely promotes pro-migratory and pro-proliferative signaling between cells. Notably, the effect of the “<100 kDa Filtrate” from both 2D and 3D have comparable effects on the KC functional activities. This suggests that the differential in activity after “Full” 3D ASC-CM treatment is likely a result of the “100 kDa” fraction and that there is likely minimal differences between the “<100 kDa Filtrate” from 2D and 3D, which is further supported by the change in secretory activity seen in FIG. 9B.

Previous studies have demonstrated how the contents of EVs can change depending on the cell type, tissue source, or donor, however; the culture conditions are often not taken into consideration. In this study, EVs derived from the exact same tissue and donor have a shift in regenerative potency when collected from ASCs from a tissue-mimetic culture system. This suggests that the ability to retain the more robust and regenerative phenotype of primary ASCs likely results in retainment of ASCs with pro-regenerative properties, where subsequent production of EVs and their contents can drive wound healing processes, such as epithelialization. Whereas in traditional 2D culture, a precipitous decline in ASC phenotypic properties ensues and the potency of EVs declines, which is highlighted by the EV dosing experiments within this study. So not only has the quantity of EVs produced in 3D increased, as previously described, but the quality has improved.

Interestingly, a closer look at the stratification and EV analyses reveal that the morphological changes exhibited by KCs after treatment with “Full” or “100 kDa” ASC-CM are potentially independent of KC migratory capacity but are also reliant on the ASC-EV fraction. This can be seen when comparing the similar relative migration of KCs after treatment with the “100 kDa” and “100-30 kDa” fractions from 3D, but only the “100 kDa” induced morphological changes. Moreover, the “<100 Filtrate” fraction is still unable to dramatically alter KC morphology even though it contains the “100-30 kDa” migratory stimulus, but addition of 2D-EVs or 3D-EVs results in the same morphological changes seen within the “100 kDa” fraction (FIG. 12E). It is also important to note that both 2D-EVs and 3D-EVs induce KC morphological changes to varying degrees, even though 2D-EVs appear to contain an anti-migratory stimulus. Similarly, the reliance of KC morphology changes on ASC-EVs is further supported by the DiI-labeling and in vitro tracking data of ASC-EVs, where the KCs undergoing the most drastic morphology changes are the ones taking up the greatest proportion of EVs.

Lastly, an EV dosing study was performed to further understand ASC-EV functionality at comparable doses and if KCs exhibited any dose-dependent wound healing responses to the EVs and/or changes in KC phenotype. Native expression of K5 (and K14) is typically considered to decline during wound healing and K16 (and K6) are considered to increase, with K10 more of an indicator of suprabasal differentiated KCs that can denote granular layer KCs when paired with increased expression of FLG. Suprabasal K10 expressing KCs are considered to switch to K16/K6 expression during the wound healing process. K16/K6 expression is thought to play a critical role in collective cellular migration of KCs. In this working example, 3D-EVs appear to significantly enhance expression of all cytokeratin markers in a dose-dependent manner. Whereas 2D-EVs led to a decline in K10 expression and formation of sporadic aggregates of K5 versus the more homogenous cellular distribution seen in 3D-EV treated KC, possibly hinting at enhanced K5 protein turnover and remodeling occurring in 2D-EV treated KCs.

Further assessment with functional and RNA analysis demonstrated a positive dose-dependent correlation of KC metabolic, proliferative, and migratory activity when treated with 3D-EVs. Whereas treatment with 2D-EVs contained a negative correlation with KC migratory activity and a positive correlation in proliferative activity. This data further demonstrates the anti-migratory stimulus for KCs found within ASC-EVs from 2D culture. Both 2D-EVs and 3D-EVs were able to promote a dose-dependent morphology change in KCs; however, 3D-EVs contained a more potent stimulus with the effects of the low dose 3D-EVs (5-μg/mL) mirroring that of the high dose 2D-EVs (250-μg/mL).

It is important to note the key morphological features changing within KCs after ASC-EV treatment that appear to happen in a stepwise manner. First, KCs begin to cluster together and form small satellite-like colonies that eventually merge into bigger colonies. Second, KCs begin to alter their cellular morphology from a rounded shape to a more irregular spindle-like shape, with rearranged cytoskeletal structures and increased cellular extensions. Lastly, KCs begin to firmly attach and integrate with one another to form a continuous cellular sheet with more flattened cellular populations. The correlation between EV/exosome uptake and formation of a nuclear actin-cap within the KC cell sheets was an interesting and unexpected finding which likely played a direct role in the morphological flattening and increased nuclear surface area discussed previously. Moreover, actin-cap formation promotes nuclear flattening and is a known modulator of gene expression and often associated with mechanotransducive and epigenetics responses. Broadly speaking, both 2D-EVs and 3D-EVs were able to induce this KC response to varying potencies. However, where 3D-EVs diverge is their enhanced capacity to stimulate migratory activity in KCs. This is the first demonstration of diverging migratory activity of ASC-EVs and its possible association with KC morphological changes. To the authors' knowledge this is the first in vitro demonstration of substantial EV-induced KC morphological changes (e.g., actin-cap and cell sheet formation) and its potential association with KC wound healing activity; thus, EV-induced KC morphological changes requires further investigation.

Interestingly, the RNA profile of the KCs treated with ASC-EVs demonstrated a diverse phenotypic profile. Both 2D-EVs and 3D-EVs demonstrated a dose-dependent response in several key KC genes, including K10, K16, FLG, TWIST1, and CCND1. However, the augmentation of KC wound healing activity was more pronounced at a functional and RNA level after 3D-EV treatment compared to 2D-EVs. Notably, 3D-EVs were able to promote increased expression of a proliferative and basal-like phenotype (CCND1 and K5), a differentiated suprabasal-like phenotype (K10 and FLG), a wound healing phenotype (K16), and a more migratory phenotype (K16 and TWIST1). Moreover, KCs treated with 2D-EVs demonstrated apparent breakdown and turnover of K5 protein (FIG. 14A), suggesting a possible transition period where the KCs were losing their basal-like phenotype. A closer look also demonstrates that 3D-EV treated KCs exhibit an EMT-like transition with increased expression of TWIST1 and an increased N-Cadherin-to-E-Cadherin ratio, which is further supported when paired with the increased vimentin production, morphological changes, and enhanced migration. Together, these data suggests that ASC-EVs from 3D are able to more effectively stimulate KCs to form a stratified collective cell sheet, consisting of EMT-like migratory KCs forming the leading edge of the cell sheets, while maintaining cell-to-cell adhesion in the interior of the cell sheet and expression of cytokeratins associated with suprabasal differentiated (K10), migratory (K16), and proliferative (K5) phenotypes.

Notably, collective cell sheet migration is a proposed mechanism of native KC re-epithelialization and to date, this process has been poorly recapitulated in vitro. It is thought that 2D monolayer culture of KCs is unable to recapitulate the proper differentiation and morphological changes necessary to promote collective cell sheet migratory activity of KCs. However, our data suggests that EVs may in fact be a missing link and key in promoting this process that is considered to occur in vivo. To the authors' knowledge this is the first demonstration of the ability of ASC-EVs to broadly promote multiple key functional phenotypes in KCs rather than just migration and/or proliferation, and provides new insight into potential native in vivo signaling dynamics of wound repair that warrants further investigations. More specifically, the apparent capacity for KCs to retain expression of key basal and suprabasal epithelial-like cytokeratin markers simultaneously, while also expressing mesenchymal-like markers (vimentin, n-cadherin, twist1) after EV treatment.

### 5. Conclusion

In conclusion, this study highlights the importance of understanding the role of culture environment on MSC-like cells, their phenotype, and their capacity to secrete regenerative compounds by performing a direct comparison of the regenerative functionality of the ASC secretome from both 2D culture and a tissue-mimetic system (e.g., TMHB system). It is important to note that MSC-like populations are dynamic and highly adaptive to their environments, thus the secretome and/or exosomes from one population may vary depending on the system utilized to generate the biologics. Therefore, the effect of any system should be evaluated before generalizing the effects of specific MSC-derived secretome components. Additionally, this study demonstrates that EV/exosomes may be a key driver of epidermal regeneration activity within the ASC secretome and, when generated within a tissue-mimetic system, are able to enhance the formation of collective, migratory cell sheets of KCs, a critical process during native epidermal regeneration. EV/exosomes offer a unique approach to improving current wound healing modalities clinically due to their enhanced stability, relative to proteinaceous biologics (growth factors), and their diverse/dynamic compositions. Thus, EV/exosomes could act as adjuvant therapies via integration into current wound dressing and/or injectable hydrogel systems in order to tailor therapies and enhance wound outcomes. Ultimately, more studies may be warranted to investigate the potential clinical benefits that MSC-derived EV/exosomes may provide, but this study helps provide new insight into the direct role of EV/exosomes on KC wound healing and epidermal regeneration.

### Example Set #2: Tailoring the Secretome Composition of Mesenchymal Stem Cells to Augment Specific Functions of Epidermal Regeneration: An In Vitro Diabetic Model

### Background and Executive Summary

Wound healing consists of a dynamic series of events that are highly dependent on paracrine factors for proper progression through the phases of wound healing. Inappropriate progression through the phases of wound healing is associated with insufficient epidermal regeneration (i.e., re-epithelialization) of wounds and subsequent propagation of chronic wounds, such as diabetic ulcers, which are associated with an increase in patient morbidity. Recently, investigation into the dynamic secretome of Adipose-derived Mesenchymal Stem Cells (ASCs), have shown promise in augmenting the wound healing response of chronic diabetic wounds. However, currently utilized 2D culture techniques are known to drastically alter the regenerative phenotype of ASCs. In this working example, a TMHB system was utilized as a means to culture ASCs. The capacity for the ASC secretome to augment epidermal regeneration activity was then evaluated after exposure of ASCs to “wound priming stimuli” in 2D and 3D. The priming stimuli consisted of coating the 2D and 3D systems with the wound matrix proteins, collagen type I, fibronectin, and fibrin. To understand the potential benefit of the ASC secretome in the context of diabetic wounds, keratinocytes (KCs) were exposed to super-physiological glucose levels to induce a diabetic-like phenotype (idKCs). Relative to KCs, idKC exhibited a 52% and 23% decline in proliferation and migration, respectively. Subsequently, analyses of the ASC secretome were performed. ASC conditioned media (ASC-CM) from tissue-mimetic culture demonstrated a >50% increase secretion of proteins and a 2-fold increase in secreted EVs, relative to 2D culture. Interestingly, the different priming stimuli did not alter the total amount of protein or EVs secreted within the tissue-mimetic system. However, evaluation of specific soluble proteins via ELISA revealed significant differences in key epidermal regeneration factors, such as EGF, IGF-1, FGF-2, MMP-1, TIMP-1, and TGFβ-1. Additionally, the relative effect of ASC-EVs from the 2D and 3D system on idKCs epidermal regeneration functionality varied significantly, with EVs from 3D-Collagen culture providing the most significant benefit on idKC activity. Together, this data supports the utilization of tissue-mimetic culture to enhance to adaptability and secretory activity of MSC-like populations in order to generate tailored biologics, via priming stimuli, for specific wound healing applications.

### 1. Introduction

Wound repair and regeneration is a complex and dynamic series of events that occurs in the setting of tissue damage and involves a diverse array of cell populations. For effective restoration of tissue function and anatomical homeostasis to occur, tightly controlled cellular and molecular signaling cascades promote migration and proliferation of cells, extracellular matrix deposition/remodeling, and cytoskeletal modulation of the local wound healing cell populations. However, many patients and wound types can be predisposed to inadequate healing and protraction of the wound healing response that can ultimately result in progression towards a chronic wound phenotype.

The epidermal barrier of the skin is instrumental in maintaining the viability of deeper tissue structures and allowing proper wound healing processes to occur. Thus, a critical step of wound healing after an injury is the process of re-epithelialization (epidermal regeneration), which is carried out by epidermal keratinocytes with the goal of reestablishing the skin's external barrier properties. Proper re-epithelialization requires keratinocytes to undergo a phenotypic switch. This phenotypic switch includes altering cytoskeletal and junctional proteins, such as cell-cell and cell-matrix junctions, to become more migratory and proliferative in order to repopulate and “close” the wound. Additionally, keratinocytes must alter their keratin expression from a more proliferative basal type (keratin 5 and 14) to expressing keratins associated with suprabasal differentiation (keratin 10) and an active wound response (keratin 16). Feedback and paracrine signaling from surrounding cell populations help orchestrate proper re-epithelialization, including critical crosstalk between dermal fibroblasts, dermal adipose, and other epidermal keratinocytes. However, this cellular communication can become dysregulated due to imbalanced paracrine signaling and result in perpetually non-healing wounds that do not close, often progressing to significant limb disease.

Chronic wounds are often a consequence of comorbid medical conditions like diabetes, where up to 15% of diabetics develop ulcerative wounds with a greater than 50% recurrence rate. Diabetic wounds inherently have an improper balance and composition of bioactive compounds within the tissue, such as depleted growth factor bioavailability, imbalanced proteolytic activity, and a pro-inflammatory cytokine profile. Moreover, high glucose levels is known to results in increased oxidative damage and glycation of cellular proteins. This propagates the abnormal progression of a number of wound healing processes, including proper keratinocyte differentiation and functional activity associated with epidermal regeneration. Notably, keratinocytes isolated from diabetic wounds have been shown to lack the same capacity to alter their keratin expressional pattern and properly migrate. Thus, diabetic wounds are associated with a cycle of sustained, inappropriate cellular signaling and inadequate neotissue formation. Consequently, lack of wound closure can result in polymicrobial infections, desiccation, and reinjury of diabetic wounds, which remain the leading cause of non-traumatic lower limb amputations with an associated 5-year mortality post-amputation ranging from 55-70%, second only to lung cancer. The principle of decreased wound healing capabilities of diabetic keratinocytes was taken advantage of within this study via generation of an inducible diabetic-like keratinocyte population through sustained exposure to high glucose levels in order to generate functional deficits in keratinocytes.

The harsh and complex nature of chronic wounds and their tissue microenvironments has also resulted in a lack of development of focused and effective therapeutic interventions, with inadequate re-epithelialization remaining a major limitation of current chronic wound therapies. Therefore, there remains a critical need to develop a therapy that can help circumvent the loss of proper keratinocyte functionality and restores the appropriate re-epithelialization capacity of wounds to prevent/revert progression of complex chronic wounds. Bioactive compounds, such as growth factors (GFs) and extracellular vesicles (EVs), coordinate tissue reparative processes and hold immense regenerative capabilities. Yet, enhanced proteolytic degradation, radial diffusion, and a lack of biological diversity from single GF administration in prior clinical trials has led to mixed results of therapies aimed at utilizing GFs for chronic wounds. However, the diverse yet balanced milieu of GFs, antioxidants, extracellular vesicles (EVs), immunomodulatory cytokines, proteases and proteolytic inhibitors secreted from adipose-derived mesenchymal stem cells (ASCs) could offer a unique approach to develop targeted therapies for treating chronic wounds that circumvents the limitations of single GF and/or autologous cell therapies.

Currently, the most common therapeutic modalities used clinically for chronic wound care are moist dressings and continuous surgical debridement. However, early initial studies with ASCs in in vivo animal models and clinical trials have shown the ability for ASCs to potentially enhance the rate of wound “closure” and improve diabetic wound outcomes. Recent studies have demonstrated that a key component of the inherent regenerative capabilities of ASCs is their secretome. ASC populations and their secretome are highly diverse and adaptable, allowing them to rapidly respond to a variety of environmental stimuli in a state-dependent manner, such as within a wound environment. Notably, both direct injection of ASCs and ASC secretory compounds into chronic wounds have previously demonstrated improved outcomes in vivo. Interestingly, hypoxia priming of ASCs has been shown to upregulate secretion of angiogenic compounds and priming with inflammatory cytokines has been shown to upregulate secretion of anti-inflammatory and mitogenic compounds from ASCs. Moreover, fibrin, a matrix compound and native wound healing stimulus, has been used as a delivery vehicle for ASCs and shown the ability to improve overall wound outcomes, including in chronic wounds. Additional matrix-derived biomaterials have also previously been investigated to control the bioactivity of MSC populations, including collagen I and fibronectin, which are both intricately involved within native wound tissue, and have demonstrated the ability to promote wound healing. Thus, ASCs demonstrate an inherent capacity to respond to environmental stimuli via modulating their secretory activity. The previous studies looking at delivery vehicles, such as fibrin, also highlight that matrix-derived substrates may also be critical in modulating the wound healing capacity of ASCs. Together, this data suggests that the wound environment contains a variety of stimuli (e.g., hypoxia, inflammation, and matrix compounds) that alter the adaptive secretion of paracrine factors from ASCs that can promote wound healing.

While previous data with MSC-derived biologics and cell therapies have demonstrated promise, to date, they have almost exclusively utilized 2D culture expansion for achieve adequate quantities of biological products (e.g., cells or secretome). Notably, the biochemical and biomechanical cues that cells are exposed to within the native tissue microenvironment are key to regulating cellular phenotype and activity, especially in mesenchymal stem cell (MSC) populations such as ASCs. Thus, culture of ASCs within an unphysiological and rigid 2D culture systems has been shown to promote a significant decline in ASC phenotype and viability. Consequently, 2D cultured ASCs lose their inherent adaptability and regenerative properties, leading to inconsistent and tainted secretory products. However, culture of ASCs within softer 3D systems that more closely resemble native tissue mechanics have been shown to retain the native ASC phenotype longer and result in secretion of more robust regenerative compounds. Thus, recent investigations have looked to develop 3D tissue-mimetic systems that enhance culture of ASC populations and permit easily tailorable properties to modulate ASC phenotypic activity (e.g., introduction of exogenous priming stimuli). To date, most data on ASC priming involves 2D culture systems or 3D spheroids, and focuses on enrichment of specific soluble factors secreted by ASCs (e.g., increased VEGF secretion), whereas data on understanding “functional” changes to the entire ASC secretome in the context of chronic wounds is still being investigated. Based on previous clinical trials and literature, we hypothesize that treatment with the complete and balanced ASC secretome will enhance keratinocyte function and epidermal regenerative activity due to the additive and synergistic effects of all secretory compounds (e.g., GFs, EVs, antioxidants, antiproteases), although certain compounds within the secretome may provide more potent stimuli in specific situations.

Due to the health status of patients with chronic (e.g., diabetic) wounds, the regenerative capacity of autologous ASCs often lacks the same ability to improve wound healing to the same extent as that seen with healthy ASCs. Thus, ASC-derived acellular byproducts offer a cell-free alternative to current cell-based regenerative therapies and have shown immense paracrine effects, including the modulation of epidermal regeneration. In this study, a tissue-mimetic 3D hydrogel system (e.g., TMHB system), mechanically analogous to native adipose tissue, was utilized for culture of ASCs and compared/contrasted to traditional 2D culture. Both 2D and 3D systems were coated with a variety of matrix-derived substrates native to wound tissue in order to assess for modulation of the ASC secretome for enhanced epidermal regeneration functional activity in keratinocytes. This study aims to provide valuable insight into additive and/or synergistic effects of priming allogeneic ASCs with different substrates while controlling for their mechanical environment via a tissue-mimetic 3D system (e.g., TMHB system). Thus, the more robust ASC populations within the same 3D mechanical environment but exposed to different matrix compounds found within wounds, will potentially provide new insight into native signaling responses of ASCs to wound tissue while also demonstrating the ability to produce tailored acellular biologics for specific applications.

### 2. Materials and Methods

### 2.1. Cell Culture

Human adipose-derived mesenchymal stem cells (ASCs; Lonza, Lot #18TL212639, 23-year-old Female, Black), human keratinocytes (KCs; Lonza, Lot #18TL318559, 62-year-old Male, Caucasian), were utilized in this study. ASCs were cultured in RoosterNourish MSC-XF (RoosterBio; Cat. #KT-016) for growth media (MSC-GM) and switched to RoosterCollect EV-Pro (RoosterBio; Cat. #K41001) for serum-free, low particulate media. DermaLife K Keratinocyte Medium Complete Kit was obtained from Lifeline Cell Technologies (Maryland, USA; #LL-0007) and used for KC culture. Traditional T-150 flasks were used for expansion until ~80% confluency was reached and subculturing (i.e., passaging) of ASCs and KCs was performed. A functional diabetic phenotype was induced in “Passage 1 (P1)” keratinocytes (KCs) via dosing KC-GM with a high dose (25 mM) of glucose for 10 days to achieve an “induced-diabetic” phenotype in keratinocytes (idKCs). An initial characterization of P1 ASC phenotype was performed, and P2 ASCs were utilized for this study in its entirety to eliminate any effect of subculturing. 2D and 3D cells were seeded at the same seeding density of ~1500 cells/cm² and 200 μL media/cm² to allow for analogous comparison between 2D and 3D culture and conditioned media. The TMHB system is ~1-cm³ and is a 3D-printed cell culture and expansion system called an X-Block (Ronawk; Kansas, USA) that contains a unique microarchitectural design that runs throughout the entirety of the TMHB that permits mass transport and nutrient exchange. The TMHBs were placed into a glass 6-well culture plate for culturing of ASCs. Coating of the 2D and 3D systems was performed 24-hours before cell seeding. Cells were then added dropwise to the surface of the hydrogels and allowed to migrate/disperse into the porous microarchitecture. Coating substrates included a thin coating of either collagen type 1 (Corning; Cat. #354265) at a concentration of 1-μg/mL, fibronectin (Corning; Cat. #356008) at a concentration of 1-μg/mL, or fibrin at a concentration of 10-μg/mL. Fibrin was fabricated via coating with 10-μg/mL of fibrinogen (Sigma; Cat. #F3879) for 24 hours, followed by 3× washes in HBSS and addition of 10 units of thrombin (Sigma; Cat. #T6884) for 30 minutes. A non-coated control was used for both 2D and 3D. All samples were then gently submerged in HBSS 3× to wash any residual substrate.

### 2.2. Assessment of ASC Phenotype

Initial assessment of adipogenic, chondrogenic, and osteogenic trilineage differentiation potential of ASCs (at P1) was performed via culture with differentiating media, according to the manufacturer's instructions. Adipogenic differentiation was performed using hMSC Adipogenic Differentiation BulletKit™ (Lonza; Cat. #PT-3004) and assessed for adipogenesis via Oil Red O assay (ScienCell; Cat. #0843). Chondrogenic differentiation was performed using hMSC Chondrogenic Differentiation Medium BulletKit™ (Lonza; Cat. #PT-3003), and was supplemented with TGF-β3 (Lonza; PT-4124) at a concentration of 10-ng/mL and assessed for chondrogenesis via Alcian Blue assay (ScienCell; Cat. #8378). Osteogenic differentiation was performed using hMSC Osteogenic Differentiation Medium BulletKit™ (Lonza Cat. #PT-3002) and assessed for osteogenesis via Alizarin Red S assay (ScienCell; Cat. #0223). Evaluation of ASC “stem-like” phenotype was also evaluated at P1 for positive immunolabeling for CD73/90/105, and negative immunolabeling for CD34/45. All primary antibodies were obtained from Abcam (Cambridge, UK) unless otherwise stated. In brief, ASCs were seeded in 2D for 24 hours, fixed in 4% paraformaldehyde for 15-minutes, washed 3× with HBSS, blocked with 2% donkey-serum, and immunolabeled for CD34 (ab81289), CD45 (ab40763), CD90 (ab181469), CD105 (ab231774). CD73 (Cat. #41-0200) was obtained from Invitrogen (Waltham, MA). Hoechst 33342 (Invitrogen; Cat. #H₃₅₇₀) and Alexa Fluor 488 Phalloidin (ThermoFisher; Cat. #A12379) were used as counterstains. ASC were cultured in 2D or 3D systems for 8 days and then were extracted for proteomic and phenotypic analyses. For ASC cell number quantification, upon extraction, cells centrifuged to pellet the cells, lysed with 0.5% Triton-X and evaluated via PicoGreen Quant-iT™ dsDNA Assay Kit (Invitrogen; Cat. #P7589), per the manufacturer's instruction.

### 2.3. Isolation of ASC Conditioned Media

ASC conditioned medium (ASC-CM) collection was performed via MSC-GM media removal, cells were then washed 3× with HBSS and serum-free MSC media was added for an additional 24-hr wash. The 24-hr wash was removed and new serum-free media was added followed by collections at 24-hr intervals for three consecutive days during days 6-8 of culture. The ratio of media/cell was standardized for all groups. Collected ASC-CM was centrifuged at 1500 g for 10-minutes to eliminate cell debris, Steriflip filtered with a 0.22-μm filter, and stored at −80° C. for long-term storage until use. ASC-CM from each day (6, 7 or 8) for each replicate (n=3) was combined to create a “batch” mixture to achieve adequate volumes for all experiments and eliminate any potential variability between ASC-CM from different media collection days.

### 2.4. ASC-CM Extracellular Vesicle (EV) Production

EVs were isolated via ASC-CM centrifugation at 4000 g for 30-minutes through a Vivaspin 20 MWCO 100,000 kDa centrifuge cutoff filter (Cytiva; Cat. #28932363) followed by washing with PBS and re-centrifugation at 4000 g for 5-minutes through the 100-kDa filter, for a total of 3 washes. EVs were precipitated from the remaining >100-kDa concentrate overnight using a ExoQuick-TC kit (SBI; Cat. #EXOTC10A-1), per the manufacturers protocol. Then EVs samples (n=3) were resuspended in PBS and aliquots were removed and used to quantify relative protein content as an indirect measure of EV content via Pierce™ BCA Protein Assay Kit (Invitrogen; Cat. #23225), QuickDrop (Molecular Devices; SpectraMax QuickDrop Micro-Volume Spectrophotometer) quantification via absorbance at 280-nm, and Pierce™ Coomassie “Bradford” Protein Assay Kit (Invitrogen; Cat. #23200). Purified EV samples were also evaluated via Nanoparticle Tracking Analysis (NTA; Malvern Panalytical; Nanosight LM10) to further assess particle concentrations and size distributions.

### 2.5. ELISAs of ASC-CM Soluble Protein

Twelve Quantikine Enzyme-linked immunosorbent assays (ELISAs) were purchased (R&D Systems) and used to quantify the concentration of twelve key proteinaceous factors within the ASC-CM that are involved in wound healing and epidermal regeneration. The twelve assays included Epidermal Growth Factor (EGF), Heparin-binding EGF (HB-EGF), Insulin-like Growth Factor (IGF-1), Fibroblast Growth Factor 2 (FGF-2), Keratinocyte Growth Factor (FGF-7), Transforming Growth Factor Beta 1 (TGF-β1), Interleukin 1 Beta (IL-1β), Interleukin 1 Receptor Antagonist (IL-1Ra), Pro-Matrix Metalloprotease 1 (MMP-1), Matrix Metalloprotease 9 (MMP-9), Tissue Inhibitor of Metalloproteases 1 (TIMP-1), Tissue Inhibitor of Metalloproteases 2 (TIMP-2). Assays were carried out per the manufacturer's instructions and only performed on the soluble proteins within the previously collected ASC-CM. If protein concentration was below limit of detection for the ELISA on first attempt, ASC-CM protein was concentrated 10× and re-ran, but if still no protein was detected, then that protein of interest was deemed too low to quantify.

### 2.6. ASC-CM Antioxidant Composition

Collected ASC-CM sample antioxidant activity was assessed with a Total Antioxidant Capacity (TAC) Assay kit (Cell Biolabs; Cat. #STA-360), per the manufacturer's instructions. The TAC kit evaluates antioxidant activity via the reduction of copper (II) to copper (I) and utilizes the naturally occurring antioxidant uric acid as a control standard. Thus, antioxidant activity was measured in mM equivalents of uric acid. Control serum-free MSC media was used to assess baseline antioxidant activity of media without exposure to cells. Assays were performed with technical replicates and biological triplicates (n=3).

2.7. KC and idKC Functional Activity After ASC-CM Treatment

KCs and idKCs were plated in 2D cultures plates and allowed to acclimate and achieve appropriate confluences (>24 hours) for each assay. KC-GM was then removed, cells were washed, ASC-CM was applied for 24-hours, and experimental assays for metabolic, mitochondrial, proliferative, or migratory activity were then performed per the manufacturer's instructions. ASC-CM was used as a “supplement” for the Keratinocyte Growth Media (KC-GM) and dosed at a 2:1 ratio (ASC-CM to KC-GM). In short, plated cells were analyzed via PicoGreen fluorescence obtained at 435/535 nm (n=3) to quantify DNA as a surrogate measurement of proliferation. PrestoBlue fluorescence was obtained at fluorescence was obtained at 560/590 nm (n=3) and displayed as an average relative fluorescent unit (R.F.U.) of PrestoBlue per Hoechst signal (350/460 nm) to obtain approximate metabolic activity per cell. KC scratch assays were performed to evaluate changes in wound size/area as a surrogate measurement of KC migration after inflicted a scratch within a confluent monolayer of KCs (n=3). Migration images were taken using an ImageXpress Micro XLS Imaging System (Molecular Devices) and the percent of wound area closed at 24-hours was determined via ImageJ analysis. Subsequently, EVs isolated from both 2D and 3D, coated and non-coated samples, were added to KC-GM at a final concentration of 150 μg/mL EV protein. Concentration of EVs was determined via previously performed protein quantification, and idKCs were then evaluated for phenotypic and functional changes in epidermal regeneration capacity (metabolic, proliferation, migration) to assess variability in quality/composition of EVs from each group.

### 2.8. Proteomic Analysis

Immunolabeling of specific proteins within cells was utilized for both ASCs and KCs to detect specific phenotypic surface (ASCs) and cytokeratin (KCs) markers and processed the same up until addition of primary antibodies. In brief, cells were washed 3× with HBSS, fixed with 4% PFA, and washed again 3× with HBSS. Cells were then incubated in a 2% donkey-serum blocking buffer, with/without 0.1% Triton-X in HBSS, for at least 1-hour, Primary antibodies were then suspended in blocking buffer and applied to cells at 4° C. overnight. All primary antibodies were obtained from Abcam unless otherwise stated and included n-cadherin (ab98952), e-cadherin (ab40772), keratin 5 (ab52635), keratin 10 (ab76318), keratin 16 (ab76416), CD34, CD45, CD73, CD90, and CD105. CD73 (Cat. #41-0200) was obtained from Invitrogen (Waltham, MA). The next day, the primary antibody solution was removed and cells were washed 3× with blocking buffer followed by application of secondary antibodies for 1-2 hours, followed by a final set of 3× washes with HBSS. Cells were counterstained with the nuclear marker Hoechst 33342. Secondary antibodies were derived in donkey against either mouse or rabbit and obtained from Invitrogen. Additional proteomic evaluation included western blotting, as described previously. In brief, KC cellular lysates were prepared using a 1× RIPA buffer with addition of a protease inhibitor cocktail (Cat. #P8340; 1:100). Protein concentrations were obtained via QuickDrop absorbance at 280 nm and were analyzed by SDS-PAGE via running protein on a SurePage 4-12% Bis-Tris gel (Genescript), transferred onto PVDF membranes, and immunoblotted. Primary antibodies were all obtained from Abcam and included CD9 (ab263019), CD63 (ab134045), CD81 (ab109201), Tsg101 (ab125011), GAPDH (ab8245), β-tubulin (ab6046), n-cadherin, e-cadherin, keratin 5, keratin 10, and keratin 16. Secondary antibodies were derived in donkey and used as previously described. Blocking buffer consisted of 2% donkey-serum in HBSS.

2.9. Quantitative Real-Time PCR (qRT-PCR) Expression Analysis

RNA was isolated and purified via an RNeasy Mini Kit (Qiagen). Only RNA with a 260/280 ratio of >1.8 q were used for this study. Cycle threshold (Ct) values were recorded and analyzed via the Delta-Delta-Ct method. qRT-PCR analysis was performed on ASCs via a wound healing array and on KCs via individually selected primers. For ASCs, both 2D and 3D cultured ASCs (with or without coatings) were collected during the experimental assays after ASC-CM collections and were assessed via an RT² Profiler™ PCR Array for Human Wound Healing (Qiagen; Cat. #330231; PAHS-121ZC-24) to evaluate expression of 84 wound healing and wound healing-associated genes. Glyceraldehyde 3-phosphate dehydrogenase (GAPDH), Beta-actin (ACTB), and Beta-2-Microglobulin (B2M) were the endogenous control genes utilized by the array. For KCs, untreated KCs and idKCs cultured in KC-GM served as a control for each group. Purity of cDNA samples was assessed with a QuickDrop spectrophotometer (Molecular Devices), with a 260/280 absorbance ratio>1.8 was designated as pure. Individual qPCR primers (Qiagen; Cat. #330001) were purchased to perform RT-qPCR on CDKN2A, CDH1, CDH2, FLG, KRT5, KRT10, KRT16, TWIST1, EGFR, VIM, TWIST1, IL1B, and CCND1. Expression of GAPDH was used as an endogenous control for all samples. All qRT-PCR samples were performed in triplicate (n=3). A qTower³ ThermoCycler from Analytical Jena was utilized for the above procedure. Moreover, SYBR Green was used for the fluorophore. The entire list of GeneGlobe IDs for KCs is listed in Table 3.

TABLE 3

| Gene | GeneGlobe |
|---|---|
| (Human) | ID# |
| CCND1 | PPH00128F |
| CDKN2A | PPH00207C |
| CDH1 | PPH00135F |
| CDH2 | PPH00636F |
| EGFR | PPH00138B |
| FLG | PPH24283A |
| GAPDH | PPH00150F |
| IL1B | PPH00171C |
| KRT10 | PPH05868E |
| KRT16 | PPH20097A |
| KRT5 | PPH02625F |
| TWIST1 | PPH02132A |
| VIM | PPH00417F |

### 2.10. Statistical Analysis

All data were reported as means with standard deviation. Comparative functional analyses of initial induced diabetic were evaluated with a One-way ANOVA. All remaining statistics, including ASC-CM assays with keratinocytes, ASC-CM protein, EV quantification, ELISAs, and RNA analyses were evaluated with a Two-way ANOVA. A minimum of three biological replicates (n=3) was used unless otherwise stated. Data was tested for normality via Shapiro-Wilk and Kolmogorov-Smirnov tests and plotted with a QQ plot. GraphPad Prism 9.4.2 software (La Jolla, CA) was used for the analyses and a p<0.05 was considered significant. ImageJ was utilized for image processing.

### 3. Results

### 3.1. Characterizing Adipose-derived Mesenchymal Stem Cell Populations.

### 3.1.1 Characterization of ASC Phenotype and Substrate Coating

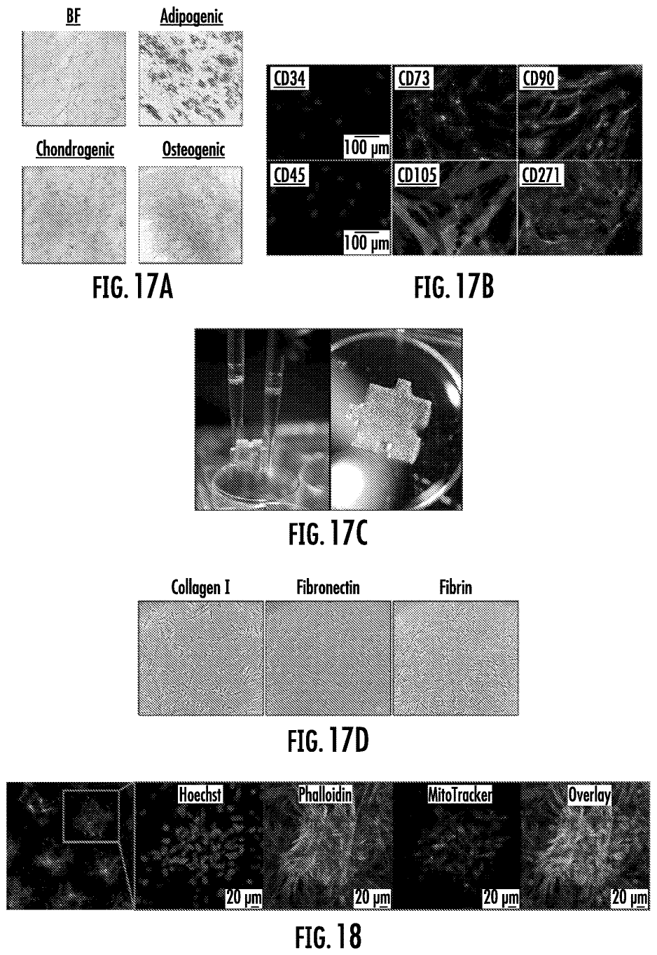

To assess for an “MSC-like” phenotype, the ASCs were initially evaluated to determined that they were adherent (FIG. 17A), exhibited trilineage multipotent potential (FIG. 17A), and expressed MSC “stem-like” surface markers (FIG. 17B), including positive staining for CD73/90/D105/CD271 and negative staining for CD34/45. Photographic images of the TMHB system were acquired to provide a visual of the macro-structure and the porous architecture (FIG. 17C). The 2D and 3D systems were then coated with different matrix-derived proteins and ASCs were cultured for eight (8) days. Throughout culture the ASCs were imaged to observe morphological changes in 2D and 3D and with the different substrate coatings (FIG. 18).

### 3.2 High Glucose Supplementation Induces “Functional” Diabetic Phenotype of KC Populations.

3.21. Induced-Diabetic Keratinocytes (idKCs) Exhibit Decreased Epidermal Regeneration Activity

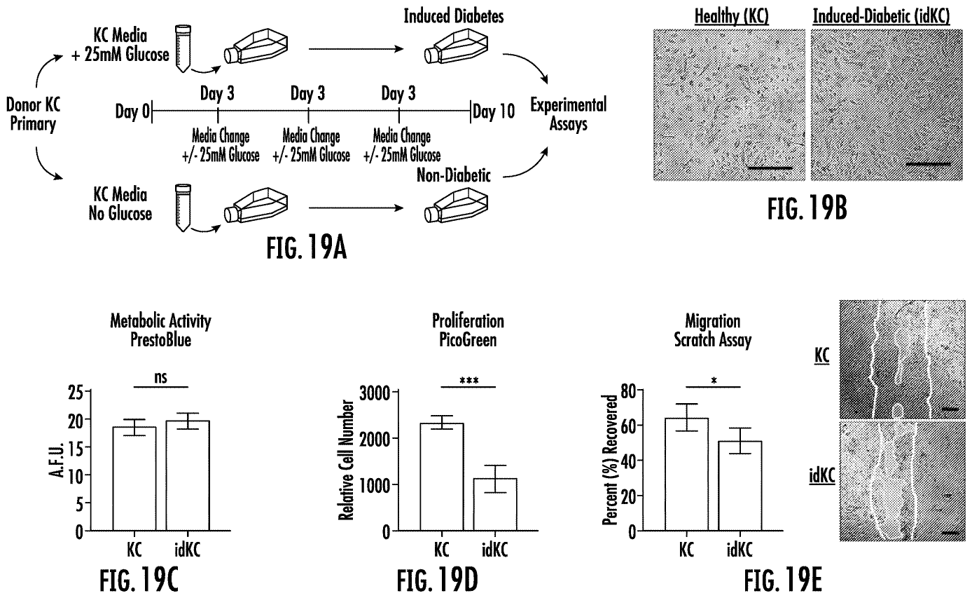

To achieve a phenotype of KCs that functionally exhibited similar properties as native diabetic keratinocytes, a prolonged culture with super-physiological glucose levels was performed (FIG. 19A). KC populations, with or without 25 mM glucose exposure, were then evaluated for changes in epidermal functional activity. Cell size of both healthy KCs and idKCs remained relatively similar, although idKCs exhibited a slight change in cell shape towards a more elongated/ellipsoid morphology, relative to the more rounded KC shape (FIG. 19B). The metabolic activity (FIG. 19C), proliferative activity (FIG. 19D), and migratory activity (FIG. 19E) were all assessed for healthy KC and idKCs. The metabolic activity of KCs and idKCs was similar; however, the proliferative and migratory activity of idKCs demonstrated significant declines within 24 hours, with a ~52% and 23% decline in proliferative and migratory activity, respectively.

3.3. Substrate-Dependent Effects on ASC Secretome have Differing Effects on idKC Functionality.
3.3.1 Matrix Substrates Alter ASC Secretion of Factors that Modulate Epidermal Regeneration Functional Activity

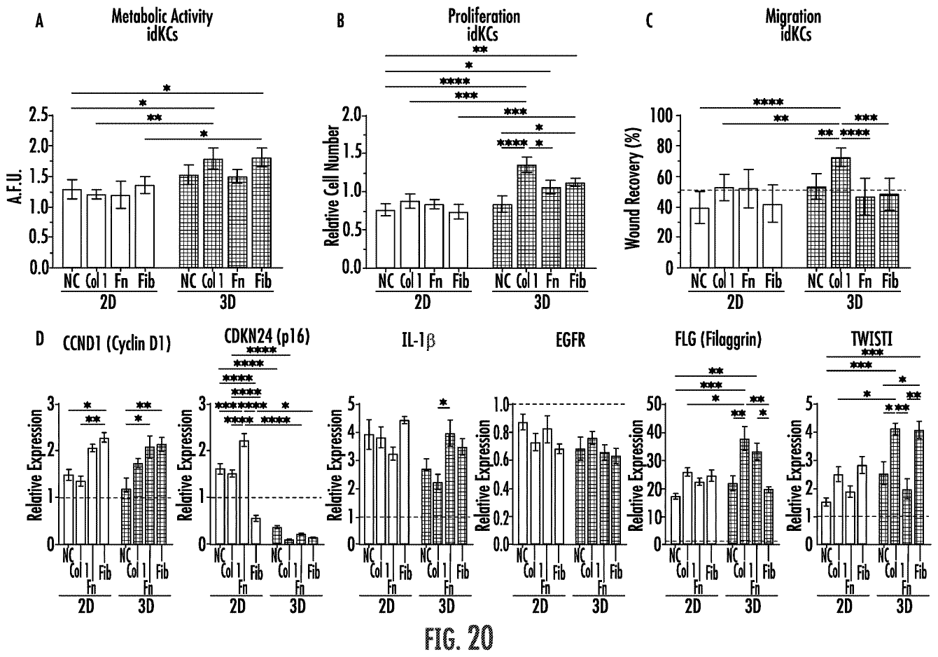

Next, the additive and/or synergistic benefits of coating materials within the 3D hydrogel system, relative to 2D, was evaluated via assessing the effect of ASC-CM on the epidermal regeneration functionality of idKCs. ASC-CM from cells cultured within the coated tissue-mimetic system had an increased propensity for enhancing the metabolic activity of idKCs, relative to 2D ASC-CM groups, with only 3D-Col (1.79-fold) and 3D-Fib (1.81-fold) resulting in a significant increase in idKC metabolic activity relative to their 2D coated counterparts (FIG. 20A). Interestingly, the non-coated 3D ASC-CM group maintained no difference in improving proliferative activity of idKCs relative to all 2D groups, whereas all coating in 3D significantly improved proliferative capacity of idKCs relative to traditional 2D-NC samples (FIG. 20B). Moreover, ASC-CM from 3D-Col (1.36-fold) and 3D-Fib (1.13-fold) significantly increased idKC proliferative capacity relative to their 2D coated counterparts, 2D-Col (0.88-fold) and 2D-Fib (0.74-fold) (FIG. 20B). Similarly, coating in 2D had no significant effect on the augmenting the ASC-CM to enhance idKC migratory activity, whereas ASC-CM from 3D-Col resulted in the greatest increase in idKC migratory activity. Moreover, 3D-Col was the only ASC-CM group to significantly alter idKC migratory activity relative to the control group, with ~73% versus ~62% percent “wound” closure in 24 hours, respectively (FIG. 20C).

Further evaluation of the idKC activity was performed via assessment of gene expression (FIG. 20D). Cyclin D1 (CCND1) was evaluated to assess for changes in idKC cell cycle and proliferation, where coating with both fibronectin and fibrin demonstrated an increased capacity to augment expression of CCND1 at 24 hours, with no differences between 2D and 3D. Similarly, the expression of EGFR and IL1B (IL-1β) in idKCs demonstrated minimal differences between 2D and 3D ASC-CM treatment, with only ASC-CM treatment from 3D-Col resulting in a significant decrease, relative to 3D-Fn, in IL1B expression. However, expression of EGFR experienced a decreasing trend in all 3D groups, whereas both 2D and 3D groups exhibited increasing expression of IL1B, relative to baseline idKC expression. Additionally, all 3D ASC-CM samples demonstrated the capacity to significantly decrease expression of p16^{ink4a} (CDKN2A), a marker for senescence, relative to their 2D ASC-CM counterparts and the baseline expression in idKCs. Moreover, 3D-Col and 3D-Fn ASC-CM both significantly increased expression of the suprabasalar marker filaggrin (FLG) in idKCs, whereas ASC-CM from 3D-Col and 3D-Fib increased expression of the migratory marker TWIST1 in idKCs (FIG. 20D).

3.3.2 Augmenting the idKC Epidermal Phenotype Via Matrix-Dependent Modulation of ASC Secretome

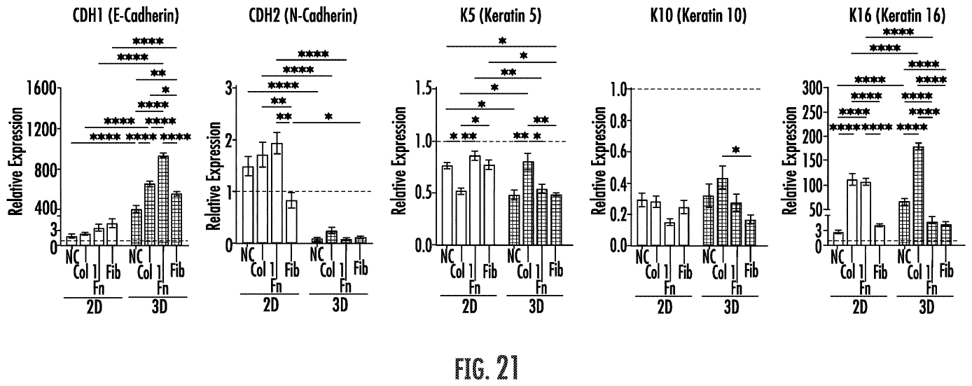

Assessment of idKC cytokeratins and cell junctional proteins were evaluated to determine changes in the epidermal phenotype of idKCs after treatment with ASC-CM via changes in gene expression with qRT-PCR (FIG. 21). Notably, was the significant enhancement in expression of E-Cadherin (CDH1) in idKCs treated with all 3D ASC-CM groups, with 3D-Fn providing the most significant stimulus. All 3D coated groups provided variable degrees of enhancement, whereas 2D coated samples provided no significant differences between coating groups. Conversely, N-Cadherin (CDH2) was significantly decreased after all 3D ASC-CM treatment groups, whereas 2D ASC-CM groups had no significant effect on idKC expression of CDH2. Interestingly, 2D-Col ASC-CM resulted in decreased expression of K5 in idKCs but 3D-Col resulted in an increased expression, relative to the other 3D ASC-CM groups. The migratory and wound responsive marker K16 was significantly enhanced in idKCs treated with ASC-CM from 2D-Col and 3D-Col, however, 3D-Col resulted in the most significant increase amongst all groups. Whereas ASC-CM from 3D-Fn and 3D-Fib resulted in a significant decrease, relative to 3D-NC. The suprabasal differentiation marker K10 exhibited minimal differences between 2D and 3D ASC-CM treatment and between different coatings, although all groups exhibited a decreased expression relative to baseline idKCs.

3.4. Matrix-Coating a Tissue-Mimetic System Enhances the Relative Secretion of Regenerative Compounds from ASCs.

### 3.4.1. Augmented Secretion of Trophic, Immunomodulatory, and Proteolytic Proteins Involved in Epidermal Regeneration

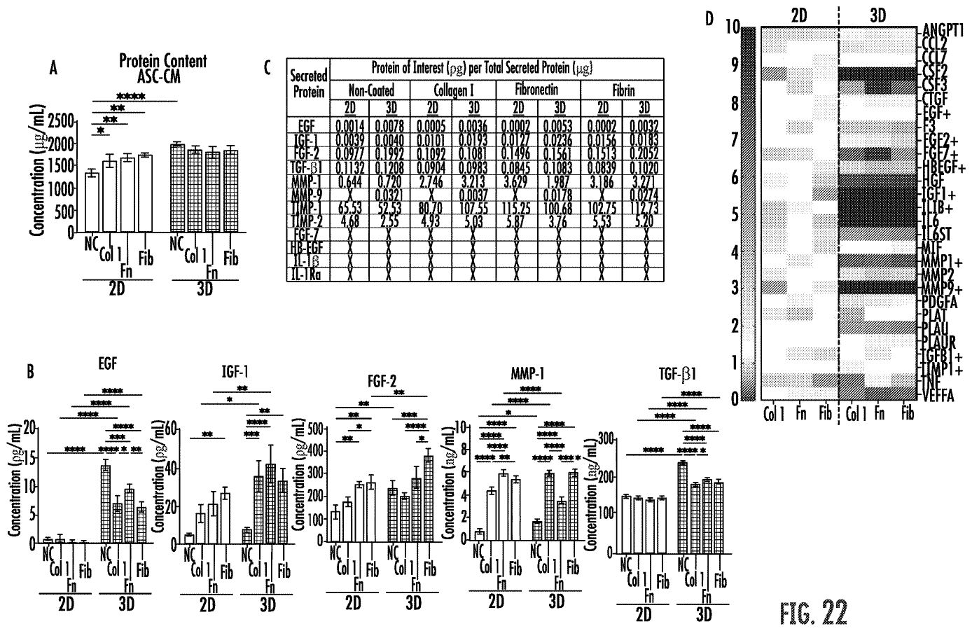

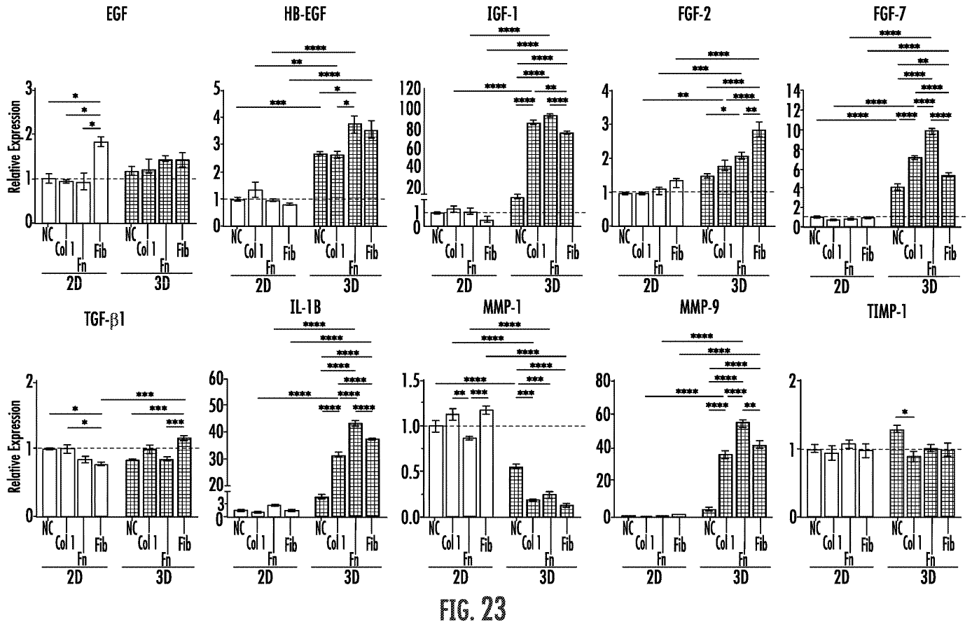

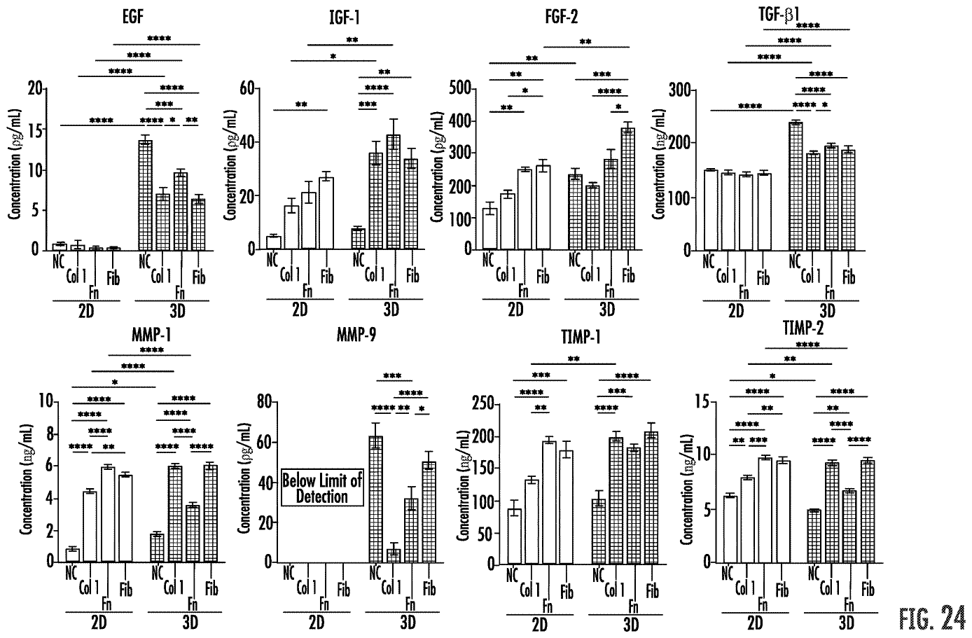

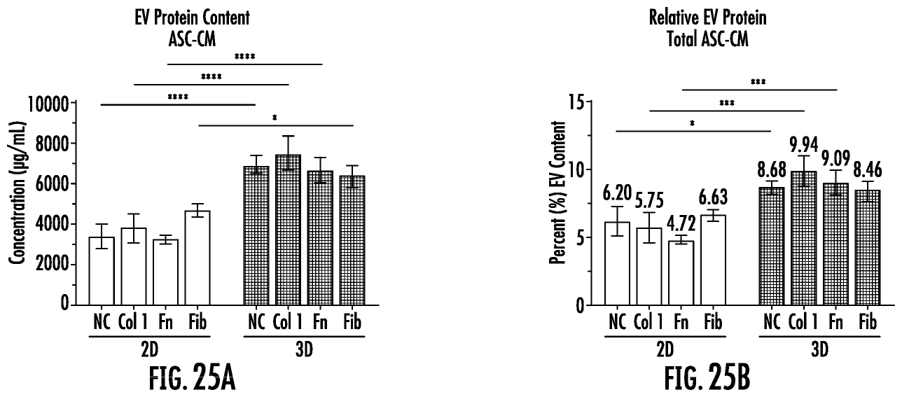

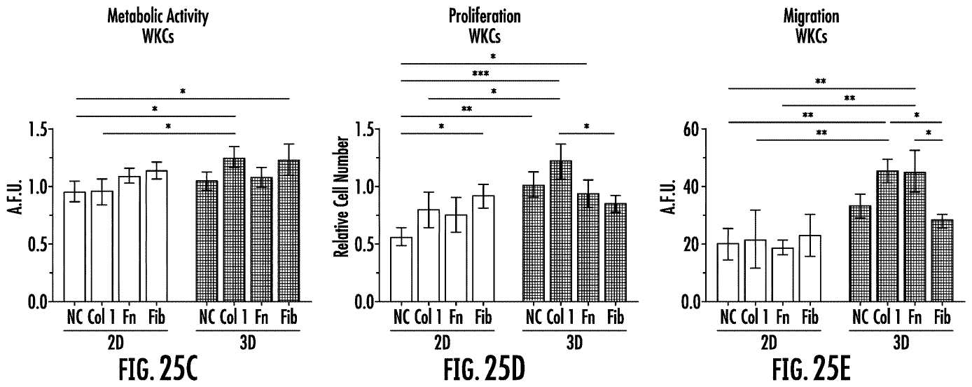

Total protein content within ASC-CM for each group was analyzed, with 2D-NC (1,341 μg/mL) exhibiting significantly less protein within the ASC-CM than all other groups. Culture within 3D-NC significantly enhanced the overall secretory activity of ASCs, with a ~48% increase secretion of proteinaceous compounds, similar to previously reported data. Interestingly, coating within 2D groups increased the secreted protein concentration to the same level of all 3D groups, whereas 3D ASC-CM exhibited no difference between coated and non-coated groups (FIG. 22A). Specific secreted protein markers known to modulate for epidermal regeneration were evaluated via ELISA and their respective concentration were quantified (FIG. 22B-C) and ASC gene expression of specific secreted factors was validated (FIG. 22D and FIG. 23). Coating in 2D groups had no effect on modulating the secretion of EGF and TGF-1, whereas coating in 2D was able to modulate the secretion of IGF-1, FGF-2, and MMP-1, 2D-Fb and 2D-Fn significantly increased the secretion of several factors. 3D culture significantly enhanced the secretion of EGF, IGF-1, FGF-2, MMP-1, MMP-9, TGF-β1, and TIMP-1, relative to 2D groups. 3D-Fib had the most significant effect on secretion of FGF-2. Additionally, all coatings significantly decreased secretion of EGF and TGF-β1 in 3D, whereas all coatings significantly increased secretion of IGF-1 and MMP-1 in 3D, relative to 3D-NC (FIG. 22B and -FIG. 24). Interestingly, 3D culture resulted in a significant decline in secretion of MMP-1 in 3D-Fn but a significant increase in the 3D-Col group, relative to their 2D counterparts. Lastly, coating in both 2D and 3D significantly increased secretion of TIMP-1, with a similar pattern in TIMP-1 and MMP-1 secretion (FIG. 22B and FIG. 24). Protein secretion from ASCs was further validated via evaluation of gene expression with a wound healing array. A heatmap demonstrates increased ASC expression of several secretory proteins in 3D relative to 2D groups, with coating in 2D having minimal effects on ASC activity (FIG. 22D). A full list of relative gene expressions for the entire array can be found in Table 4.

TABLE 4

| | Collagen I | | F [symbol] | | [symbol] | | | Collagen I | | F [symbol] | | [symbol] | |
|---|---|---|---|---|---|---|---|---|---|---|---|---|---|
| [symbol] Gene | 2D | 3D | 2D | 3D | 2D | 3D | Gene | 2D | 3D | 2D | 3D | 2D | 3D |
| ACTA2 | 1.02 | 2.36 | 1.31 | 1.66 | 0.89 | 1.64 | I [symbol] | 0.72 | 1.83 | 1.0 [symbol] | 1.69 | n/a | 0.66 |
| ACTC1 | 0.42 | 15. [symbol] | 1.05 | 9.34 | 1.77 | 7.09 | I [symbol] | 0.73 | 54.52 | 1.16 | 53.56 | 0.70 | 4 [symbol] .91 |
| A [symbol] | 0.79 | 1.70 | 0.79 | 2.06 | 0.82 | 1.85 | I [symbol] | 0.82 | 0.49 | 1.10 | 0.44 | 0.83 | 0.39 |
| CC [symbol] | 0.84 | 2. [symbol] 4 | 1. [symbol] 1 | 1.93 | 0.97 | 2.09 | I [symbol] | 0.70 | 2.37 | 0.80 | 2.8 [symbol] | 0.80 | 2.75 |
| CC [symbol] | 1.41 | [symbol] .19 | 1.35 | 0.96 | 0.38 | 1.54 | I [symbol] | 1.38 | 0.29 | 1.11 | 0.29 | 1.29 | 0.25 |
| CF [symbol] | n/a | n/a | n/a | n/a | n/a | n/a | I [symbol] | 1.21 | 0. [symbol] 1 | 0.82 | 0.25 | 1.04 | 0.22 |
| CDH [symbol] | 1.49 | 2.98 | 0.32 | 1.47 | 0.67 | 0.75 | I [symbol] | 0.81 | 0.38 | 0.94 | 0.29 | 0.90 | 0.29 |
| COL [symbol] | 1.04 | [symbol] .61 | 1.69 | 2.22 | 3.07 | 1.82 | I [symbol] | 0.85 | 0.54 | 0. [symbol] 0 | 0.53 | 0.73 | 0.52 |
| COL [symbol] | 0.94 | 3.13 | 1.05 | 4.25 | 3.07 | 3.23 | I [symbol] | 1.31 | 0.26 | 1.04 | 0.24 | 1.13 | 0.37 |
| COL [symbol] | 0.68 | 2.40 | 0. [symbol] 5 | [symbol] .26 | 0.72 | 2.88 | I [symbol] | 1.0 [symbol] | 1.0 [symbol] | 1.08 | 1.09 | 1.01 | 1.10 |
| COL3A1 | 0.63 | 3.38 | 1. [symbol] 0 | 5.96 | 0.92 | 5.53 | I [symbol] | 0.97 | 0. [symbol] | 1.09 | 0.9 [symbol] | 0.85 | 0.90 |
| COL4A1 | 0.83 | 0.20 | 0.98 | 0.14 | 0.88 | 0.17 | I [symbol] | 1.04 | [symbol] | 0.94 | 0.88 | 0.92 | 0.94 |
| COL4A [symbol] | 1.45 | 1.10 | 1.04 | 1.27 | 0.92 | 5.53 | I [symbol] | 1.06 | 0.72 | 1.03 | 0.93 | 1.10 | 0.70 |
| COL5A1 | 0.83 | 4. [symbol] 3 | 0.83 | 4.60 | 0.85 | 4.94 | I [symbol] | 0.97 | 0.60 | 1.28 | 0.46 | 2. [symbol] 3 | 0.60 |
| COL5A2 | 0.90 | 3.02 | 1.27 | 4.32 | 1.14 | 3.81 | M [symbol] | 0.99 | 0.49 | 1.10 | 0.42 | 0.93 | 0.39 |
| COL5A3 | 1.18 | 4.70 | 1.42 | 5.75 | 1.14 | 5.78 | M [symbol] | 0.99 | 0.53 | 1.05 | 0.57 | 1.12 | 0.55 |
| C [symbol] | 5.22 | 10.18 | 2.4 [symbol] | 85.00 | 1.71 | 101.67 | M [symbol] | 0.86 | 1. [symbol] 3 | 0.94 | 1.46 | 0.79 | 1.66 |
| C [symbol] | 1.06 | 4.5 [symbol] | 0.78 | 9.23 | 1.33 | [symbol] .04 | M [symbol] | 1.14 | 0.20 | 0.87 | 0.25 | 1.18 | 0.14 |
| C [symbol] | 0.94 | 1.5 [symbol] | 1. [symbol] 9 | 1.84 | 0.91 | 1.52 | M [symbol] | 0.87 | 2.50 | 1.14 | 3.45 | 0.98 | 2.90 |
| C [symbol] | 0.98 | 0.61 | 0.9 [symbol] | 0.40 | 0.97 | 0.46 | M [symbol] | n/a | n/a | n/a | n/a | n/a | n/a |
| C [symbol] | n/a | n/a | n/a | n/a | n/a | n/a | M [symbol] | 0.49 | 36.93 | 1.09 | 55.37 | 2.02 | 42.70 |
| C [symbol] | 0.81 | 0.86 | 1.2 [symbol] | [symbol] .39 | 0.96 | 1.24 | P [symbol] | 1.22 | 2.55 | 0.88 | 1.98 | 0.99 | 1.59 |
| C [symbol] | 1.07 | 0.24 | 1.08 | 0.20 | 0.78 | 0.32 | P [symbol] | 0.86 | 0.59 | 0.72 | 1.01 | 0.94 | 1.11 |
| C [symbol] | 1. [symbol] 1 | 0.56 | 1. [symbol] 7 | 0.82 | 1. [symbol] 2 | 0.75 | P [symbol] | 1.41 | 0.49 | 1.02 | 0.45 | 1.20 | 0.37 |
| C [symbol] | n/a | 2.89 | n/a | 1.75 | n/a | n/a | P [symbol] | 1.21 | 1.32 | 1.01 | 1.80 | 1.20 | 1.69 |
| C [symbol] | 1. [symbol] 3 | 0.9 [symbol] | 1.24 | 1.38 | 1.06 | 1.35 | P [symbol] | n/a | n/a | n/a | n/a | n/a | n/a |
| C [symbol] | 0.98 | 0.93 | 0.7 [symbol] | 0.98 | 1.39 | 0.61 | P [symbol] | 0.96 | 0.90 | 1.01 | 0.9 [symbol] | 0.92 | 0.80 |
| E [symbol] | 0.95 | 1.20 | 0.9 [symbol] | 1.42 | 1.82 | 1.43 | P [symbol] | 1.19 | 8. [symbol] 1 | 0.75 | 10.78 | 0.74 | 15.25 |
| E [symbol] | 1. [symbol] 5 | 1.24 | 1.08 | 1.48 | 0.98 | 1.53 | R [symbol] | 0.81 | 0.58 | 0.86 | 0.45 | 0.86 | 0.40 |
| F [symbol] | 1.46 | 3.50 | 0.89 | 2.24 | 5.40 | 2.76 | R [symbol] | 0.92 | 0.65 | 0.99 | 0.60 | 0.83 | 0.47 |
| F [symbol] | 1.07 | 0.69 | 0.78 | 0.69 | 1.01 | 0.62 | S [symbol] | 1.62 | 1.52 | 0.73 | 1.90 | 1.00 | 1.85 |
| F [symbol] | n/a | n/a | n/a | n/a | n/a | n/a | S [symbol] | 0.94 | 1.35 | 1.08 | 1.3 [symbol] | 1.05 | 1.17 |
| F [symbol] | n/a | n/a | n/a | n/a | n/a | n/a | T [symbol] | 0.9 [symbol] | 5. [symbol] 1 | 1.14 | 4. [symbol] 1 | 0.85 | 4.25 |
| F [symbol] | 0.98 | 1.79 | 1. [symbol] 2 | 2.07 | 1.36 | 2.85 | T [symbol] | 1. [symbol] 3 | n/a | n/a | 1.88 | n/a | n/a |
| F [symbol] | 0.76 | 7.13 | 0.81 | 9.87 | 0.93 | 5.37 | T [symbol] | 1. [symbol] | 1.00 | 0.85 | 0.86 | 0.79 | 1.17 |
| H [symbol] | 1.38 | 2.64 | 0.97 | 3.77 | 0.86 | 3.56 | T [symbol] | 0.84 | 0.51 | 1.08 | 0.60 | 0.89 | 0.53 |
| H [symbol] | 0.88 | 0.08 | 1. [symbol] | 0.04 | 0.75 | 0.03 | T [symbol] | 0.94 | 0.90 | 1.08 | 1.02 | 0.98 | 1.00 |
| I [symbol] | n/a | n/a | n/a | n/a | n/a | n/a | T [symbol] | 0.74 | 0.31 | 0.77 | 0.9 [symbol] | 0.45 | 0.67 |
| I [symbol] | 1.34 | 85.08 | 1. [symbol] | 92.25 | 0.49 | 75.22 | V [symbol] | 1.33 | 5.9 [symbol] | 0.91 | 7. [symbol] 4 | 0.89 | [symbol] .82 |
| I [symbol] | [symbol] | 13.93 | 2.44 | 23.20 | n/a | 35.22 | V [symbol] | 0.8 [symbol] | 0.63 | 0.70 | 0.65 | 0.69 | 0.56 |
| I [symbol] | 0.72 | 31.44 | 1.74 | 43.44 | 1.07 | 37.45 | [symbol] | 0.73 | 4.99 | 0.95 | 4.0 [symbol] | 0.86 | [symbol] .57 |
| I [symbol] | n/a | n/a | n/a | n/a | n/a | n/a | [symbol] | 1. [symbol] 6 | 0.89 | 0.82 | 0.80 | 1.03 | 0.78 |

[symbol]

indicates data missing or illegible when filed

### 3.5. Collagen Type I Coating of Tissue-Mimetic Enhances Epidermal Regeneration Capacity of ASC-EVs.

3.5.1 ASC Exposed to Collagen type I in 3D Enhance Epidermal Regeneration of idKCs Via Secretion EVs

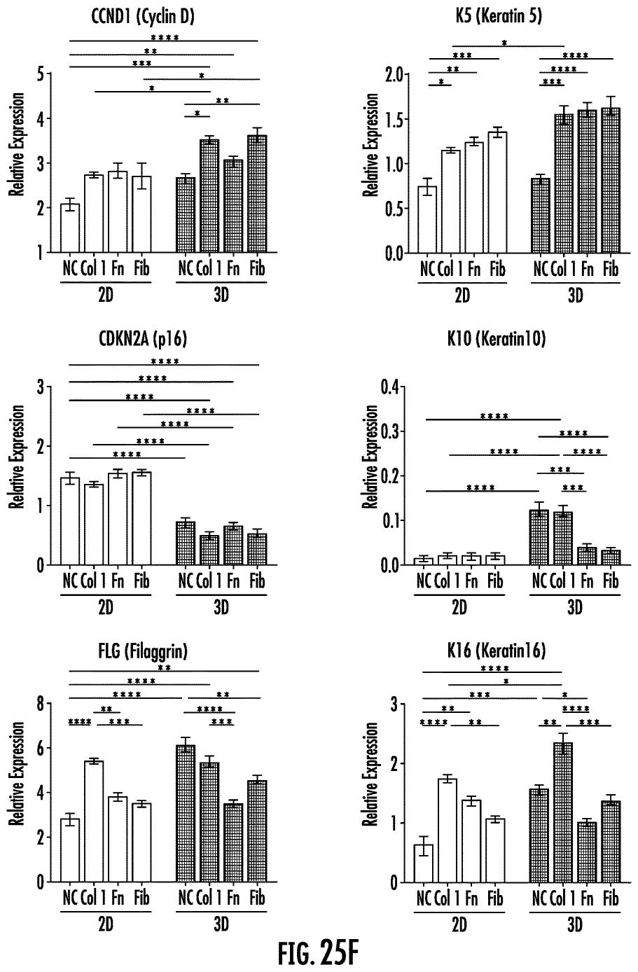

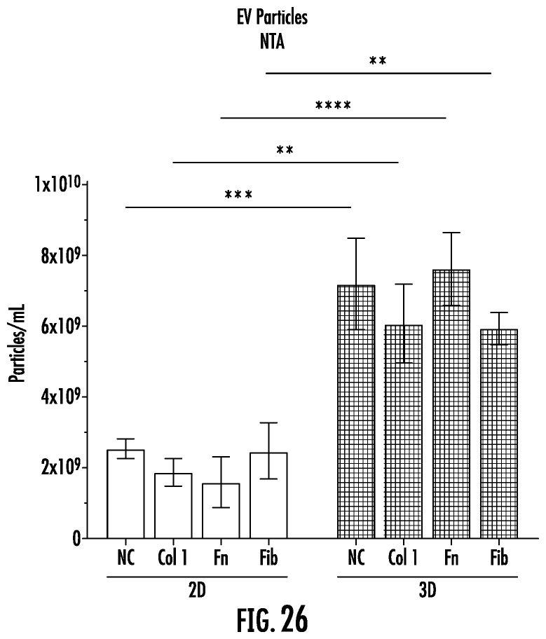

ASC-EV quality was then evaluated for ability to modulate idKC activity. ASCs within the TMHB system demonstrated enhanced secretion of extracellular vesicles (EVs), relative to 2D, for all non-coated and coated samples (~1.89-fold increase in 3D). However, substrate coating in 2D and 3D did not alter the relative concentration of EVs within ASC-CM for each respective group (i.e., intragroup comparisons) (FIG. 25A). Moreover, when controlling for total protein secreted within ASC-CM, the relative composition of EV Protein-to-Total Secreted Protein was significantly greater in 3D ASC-CM groups, relative to their 2D counterparts, except for fibrin (FIG. 25B). Analysis of the size distribution of the EVs was performed via NTA and demonstrated that >95% of all 6 particles analyzed were within the 25-250 nm range and about 2-3 times greater particles in 3D relative to 2D, similar to the EV protein quantification data (FIG. 26). Additionally, the presence of exosomal proteins CD9, CD63, CD81, and Tsg101 suggest that the EVs are exosomal in origin.

To assess the role of ASC-EVs on the previous benefits of ASC-CM treatment, idKCs were treated with KC-GM media dosed at a concentration of 150 μg/mL of isolated EVs from each respective 2D and 3D group. The idKCs were then evaluated for epidermal functional activity via assessment of metabolic (FIG. 25C), proliferative (FIG. 25D), and migratory activity (FIG. 25E). Notably, EVs from 3D-Col were the only 3D-EVs to result in a significant enhancement in all three idKC functions (metabolic, proliferative, and migratory activity), when comparing each respective 3D coating group to their 2D counterpart (FIGS. 25C-E). More specifically, 3D-Col EVs resulted in the highest increase in idKC proliferative activity, with about a 2-fold increase relative to 2D-Col (FIG. 25D), whereas both 3D-Col and 3D-Fn EVs resulted in a significant increase in idKC migration relative to their 2D counterparts (FIG. 25E).

Gene expression analysis was then performed on idKCs after 24 h of ASC-EV treatment (relative to control idKCs) to further validate idKC functional and phenotypic changes (FIG. 6F). Coating in 2D was able to result in production of ASC-EVs that enhanced the expression of K5, K16, and FLG, relative to non-coated groups, with 2D-Col exhibiting significantly greater propensity for increasing expression of K16 and FLG (FIG. 25F). However, 3D-NC resulted in a significant improvement in idKC expressional profile for all markers except K5, relative to 2D-NC. Notably, 3D coating demonstrated the capacity to modulate ASC-EV activity and improve expression of several idKC markers and resulted in a significant decrease in expression of CDKN2A, relative to all 2D-EV groups. Additionally, 3D-Col was the most consistent group to significantly impact idKC gene expression and was the only 3D-EV group to significantly enhance the expression of all three keratins, K5, K10, and K16, relative to their 2D counterpart (FIG. 25F).

### 4. Discussion

Incomplete and failure for wounds to “close” efficiently due to trauma, surgery, acute or chronic disease, and radiation-induced tissue damage often results in the progression of chronic, non-healing wounds and affects millions of people every year. Notably, diabetic wounds are among the most prevalent of all chronic wounds and currently affects ~15% of all diabetics in the US. With the aging population and rise in diabetes, the prevalence of chronic diabetic wounds is expected to increase. Thus, therapies tailored for diabetic wounds are a highly researched topic. However, currently there is minimal research investigating therapies for improving the epidermal regeneration capacity within diabetic wounds specifically. Therefore, there remains a critical need to develop a therapy that can circumvent the loss of proper keratinocyte functionality and restores the appropriate epidermal regeneration capacity of wounds to prevent further progression of chronic diabetic wounds.

Early studies with MSC populations in in vitro and in vivo wound models have demonstrated the ability to promote pro-wound healing activity in a number of key wound healing cells populations, and modulate the epidermal regeneration activity of wounds. Moreover, in vivo animal models and clinical trials with diabetic wound models have shown the ability for ASCs to directly augment wound healing, enhance the rate of wound “closure” and improve overall outcomes. The injection of ASC and ASC-loaded wound dressings for treatment of diabetic ulcers have demonstrated promise. However, retainment of ASCs within the wound site via utilization of a wound dressing or other delivery vehicle appears to be critical for enhancing therapeutic benefits with ASC therapies. This is thought to be, in part, a result of the proximity of the “wounded tissue stimulus” to ASCs, rather than the radial diffusion and cell death that is commonly seen in injected cell therapies, which ultimately allows the ASCs to continuously adapt/respond to the evolving wound tissue environment.

Although promising, cell-based therapies do have their limitations and can be highly variable depending on the baseline viability and robustness of the ASC source, which is often compromised in patients exhibiting chronic wounds. Recent investigations into the adaptive and secretory nature of ASC populations to priming stimuli have provided a unique opportunity for developing regenerative wound therapies that are capable of circumventing many of the limitations of autologous cell therapies. It is conceivable that the regenerative capacity seen from prior ASC therapies are, in part, due to the secretion of various paracrine compounds from ASCs that contain anti-oxidants, anti-inflammatory, anti-proteolytic, and targeted EV compounds. However, to date, most therapies are investigating the utilization of 2D cultured MSC populations, which is known to result in decreased viability, terminal differentiation, and a loss of regenerative capabilities, which drastically hinders the translatability of potential MSC-derived regenerative therapies.

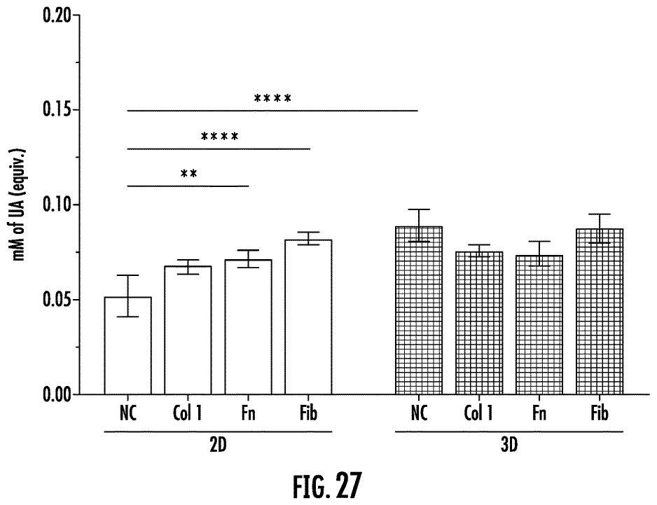

Based on prior studies that have demonstrated the ability for 2D cultured ASCs to enhance wound healing activity, in this study, a unique tissue-mimetic 3D system was utilized and compared to a traditional 2D system. The 3D system (e.g., TMHB system) is mechanically similar to native adipose tissue and thus more physiological for ASC populations. Therefore, this study uniquely investigated the effect of multiple wound matrix coatings on the ASC secretome functionality while controlling for the mechanical input from 2D and 3D. The utilization of different matrix-derived proteins used as priming stimuli in both 2D and 3D systems directly assessed for the role of the wound matrix in altering ASC secretory activity and subsequent modulation of the epidermal regeneration capacity of the ASC secretome. Based on prior studies that have demonstrated how this tissue-mimetic system helps maintain a more regenerative ASC population, the hypothesis was that a more robust ASC population will be more responsive to priming stimuli and secrete higher concentration of specific pro-regenerative factors accordingly, relative to 2D culture. This was supported in this study multiple ways, including the increased secretion of soluble protein (FIG. 22A), EVs (FIG. 25A), and anti-oxidants (FIG. 27) by ASCs within the 3D system, relative to 2D. Priming within the 3D system consequently led to greater diversification of the ASC secretome functionality and composition, compared to the 2D secretome which saw less overall diversity between different coatings.

The matrix coatings within this study were selected based on the following principles. Fibrin has a native role in wound healing and has been shown to improve wound outcomes in vivo. Fibrin has demonstrated the ability to further enhance wound healing outcomes of MSC-based treatments in vivo, including complex diabetic wounds and has been shown to promote the secretion of several specific soluble factors, including VEGF, EGF, and FGF. Fibronectin is a critical component to the extracellular matrix and basement membrane, directly interacts with collagen during wound healing, and exhibits high turnover during cutaneous wound healing due to frequent damage to the basement membrane. Lastly, collagen type I is the most abundant matrix protein in tissue, including skin, and plays a key role in wound healing and epidermal regeneration. Notably, collagen type I makes up a substantial portion of the dermal collagen that becomes exposed upon wounding, which is considered to provide a key migratory stimulus for wound healing cells. Moreover, a number of studies have investigated the benefit of encapsulating MSC populations in collagen type I and have demonstrated improved wound healing outcomes. Thus, these three matrix proteins were chosen because they provided a diverse array of substrates involved with different components of native wound healing. Future studies will investigate the role of other substrates as well as heterogenous mixtures of substrates paired with additional environmental or biochemical stimuli (e.g., hypoxia) as a means to generate a tissue-mimetic in vitro system capable of generating tailored compositions of factors for specific wound applications.

Previous studies have shown that hyperglycemia in native diabetic patients can induce a phenotypic switch in KCs altering their barrier functionality, while also inhibiting proper migration and proliferation activity. Additionally, studies show that a “functional” diabetic KC phenotype can be induced in vitro by high-dose glucose exposure to KCs, with similar cellular changes seen in KCs from a patient with diabetic wounds, including decreased proliferation and migration (FIG. 19). An inducible system was utilized in this study in order to allow for a non-diabetic control KC population to be performed in parallel. Whereas primary KCs from a diabetic patient neither guarantees functional deficits in wound healing nor allows for a non-diabetic control from the same patient.

ASC-CM assays with idKCs revealed that coating with collagen type I within the tissue-mimetic system enhanced the epidermal regeneration functionality of the ASC secretome more significantly than fibrin or fibronectin, for both 2D and 3D (FIG. 20A-C). Interestingly, ASC-CM from 3D-Fib provided a stronger metabolic and proliferative stimulus to healthy KCs, rather than 3D-Col (FIG. 17). These data provides new insight and indicates a possible shift in sensitivity of what factors may be necessary to achieve epidermal regeneration in healthy versus diabetic KCs, further highlighting the importance of tailored therapies for specific wound applications. The four highest increased differentiation markers of 3D ASC-CM treated idKCs were e-cadherin, filaggrin, twist1, and k16, all of which are critical responsive genes for epidermal regeneration. Of note, ASC-CM from 3D-Col and 3D-Fn both resulted in the two highest upregulations of filaggrin and e-cadherin, key proteins involved in barrier functionality within the suprabasal epidermis, as well as cell sheet formation (FIG. 20D). However, only 3D-Col ASC-CM resulted in a significant increase in expression of the basal marker (keratin 5), the suprabasal differentiation marker (keratin 10), and migratory and wound responsive marker (keratin 16) in idKCs, relative to other coatings (FIG. 21A-C). 2D-Col was only able to promote increased K16 expression, to a lesser extent, relative to the other 2D coatings. Together these data suggests that ASC-CM from 3D-Col had the highest capacity overall for augmenting a diverse and regenerative KC phenotype that may be associated with the improved barrier formation and functional activity seen in idKCs, and was the only group to significantly enhance the migratory capacity of idKCs relative to their baseline state (FIG. 20C).

ASC-CM from all 3D groups did demonstrate increased production of key signaling factors, relative to their 2D counterparts. Interestingly, although 3D-Col resulted in the greatest capacity to augment idKC functional activity, 3D-Col did not result in the highest secretion of any key soluble proteins that are considered to be critical for epidermal regeneration functionality, including EGF, IGF-1, FGF-2, or FGF-7 (KGF). This could suggest a few things, that the relative composition and stoichiometric balance between several factors may be important, idKCs may rely on other proteins not tested, or non-proteinaceous factors may play a more significant role in idKC regenerative activity, such as EVs. Notably, the concentration of EVs secreted by ASCs was increased for each coating group in 3D relative to their 2D counterparts, but no difference was noted between 3D groups. Suggesting the increased production of EVs was likely dependent on the tissue-mimetic environment and not the coatings. This data further supports the concept that ASCs maintain a greater propensity for regenerative secretory activity in 3D, whereas exogenous factors and/or coating substrate can be used to fine-tune and tailor the relative composition of the secretome.

To test whether the EV fraction was driving any of the functional changes seen in the idKCs, a study was performed where KC-GM was dosed with 150 μg/ml with EVs to allow for evaluation of the functional quality of the EVs on an EV-to-EV basis. Upon controlling for total amount of EVs, idKCs exhibited varying functional changes after ASC-EV treatment within 24 h. Both idKC metabolic and proliferative activities were significantly enhanced with 3D-Col relative to other ASC-EV groups. Additionally, both 3D-Col and 3D-Fn significantly increased the migratory capacity of idKC. Therefore, 3D-Col was the only 3D coating to augment idKC functional activity across all three assays, relative to their 2D counterparts.

Interestingly, the effect of ASC-EV treatment on idKC metabolic and proliferative activity exhibited a similar pattern as the data from “Full/Complete” ASC-CM treatment in FIG. 20. Suggesting that idKCs metabolic and proliferative activity may be more significantly impacted by EV entities rather than secreted proteinaceous factors. Conversely, 3D-Fn did not have a significant impact on idKC migratory activity originally when using the “Full/Complete” ASC-CM in FIG. 20. Although the migratory activity was significantly enhanced with 3D-EVs, the relative extent of migration in 24 h was less with only ASV-EV (3D-Col was ~44%) treatment when compared to the “Full/Complete” ASC-CM (3D-Col was-75%). This suggests that there is likely an additive or synergistic effect between ASC-EVs and non-EV soluble factors in augmenting idKC migratory capacity. Together these data reveal that the quality/composition of contents within the EVs varies in a substrate-dependent manner, with 3D-Col derived EVs having the highest propensity to consistently augment epidermal regenerative functionality in idKCs. The effect of EV dosing on idKC gene expression also demonstrated that 3D-EVs were able to significantly modulate idKC expressional patterns to a greater extent than 2D-EVs. For example, CCND1 and CDKN2A expression in idKCs was significantly altered after treatment with 2D and 3D EVs. Therefore, ASC-EVs may play a key role in modulating the relative progression of the idKC cell cycle, demonstrated by increased expression of the proliferation marker CCND1 after 3D-Col and 3D-Fib EV treatment, as well as how all 3D-EVs significantly reduced the senescence marker CDKN2A. The significant decline in CDKN2A relative to control idKC expression suggests that ASCs in a tissue-mimetic system may be secreting EVs containing senescence-protective compounds. Next, the suprabasal differentiation marker, FLG, exhibited similar expression patterns between ASC-EVs from the 2D and 3D coated groups, whereas the 3D-NC resulted in a significant increase in expression relative to 2D-NC. Thus, the tissue-mimetic properties of the 3D system are likely broadly beneficial in modulating FLG expression, but the benefits of 3D system are negated by the addition of matrix-derived compounds which appear to have a stronger impact on EV functionality towards altering FLG, demonstrated by the similar idKC expression profiles after treatment with either 2D or 3D EVs from coated samples. Lastly, ASC-EV were able to modulate the expression of idKC keratins, with 3D-Col being the only 3D coating group to significantly increase all three keratin markers relative to the respective 2D counterparts. This included the key “wound responsive” keratin, K16. These data were similar to the expression analysis performed on “Full/Complete” ASC-CM in FIG. 21, where 3D-Col was superior to all other groups in promoting K16 expression. The superior idKC functionality after treatment with 3D-Col EVs may be related to the capacity to modulate K16 activity in idKCs, but will require further investigations.

The EV data within this study potentially suggest that KCs may become more dependent on signaling factors found within EVs when assuming a diabetic-like phenotype. To the authors' knowledge, this is the first demonstration of diverging effects of ASC-derived EVs based on substrate coatings and the potential shift in sensitivity between external signaling molecules when KCs assume a diabetic-like phenotype. The dynamic mechanisms at play require further extensive investigations, although one possible explanation could be that the signaling dynamics shifts towards favoring uptake of EV-like particles in diabetic KCs as a result of a change in cell surface receptors. This could potentially be due to cellular and protein damage caused by super-physiological glucose levels and increased oxidative activity, thus the sensitivity of diabetic KCs to external signaling factors may be altered. Additionally, as we can see from the ASC secretome effects from the 2D system, there is much less heterogeneity in functional effects on the idKCs relative to the coated 3D ASC secretome. This indicates that not only is there a coating/material effect but likely also a combinatorial effect with the 3D hydrogel system on ASC phenotype and functionality. Moreover, the coating effect on the ASC secretome may be further augmented because the ASCs in 3D are in a more tissue-mimetic environment and, thus, are retaining their inherent adaptive nature and able to more appropriately respond to the coating stimuli.

There are elements within this study that are limiting in their current form and require further investigations, but do not ultimately detract from the overall findings. ASC populations may exhibit donor-specific variability in their regenerative properties that may alter the secretory activity from one to the next. However, the comparative study design between 2D and 3D, as well as between different coatings, allowed for any donor specific effects to impact each group to the same extent. Similarly, the effects of the ASC-CM were only tested in an “induced-diabetic” KC model. Although this allowed for healthy KCs to be tested in parallel, there are likely still differences in KC populations isolated from chronic diabetic wounds at different stages of the chronic wound progression. Additionally, future studies with other wound healing cell populations should be performed to establish a more holistic idea as to the role of ASC secretome components on diabetic wound healing. Although the utilization of only in vitro analyses can also be limiting, the utilization of a wide-range of functional, proteomic, and RNA assays provided a holistic perspective that provided data that can be used for a future in vivo study designs and provide the framework for tailoring the composition of the ASC secretome. Notably, previous studies have already established the similarity between the in vitro diabetic model used in this study and the “functional” phenotype of KCs isolated from diabetic wounds. Additional investigations into the dynamic and interrelated role of substrate/coating materials and the X-Block hydrogel system utilized is also warranted. The authors speculate that the ASC phenotype is shifting when exposed to the coating materials due to retainment of their inherent adaptive nature, however, to what extent remains in question and will require follow up studies. Lastly, although outside the scope of this study, future studies into the dynamic array of secretome compounds, including mass spectroscopy of proteins and investigations into the contents within EV (e.g., proteins, ROS, nucleic acids, miRNA), should be further investigated to better understand how certain environmental conditions shift the secretome composition. It is possible that the same key proteinaceous compounds that drive healthy epidermal regeneration still promote epidermal regeneration in diabetic KCs, but are packaged within EVs instead of secreted as soluble proteins, and thus were not detected via the ELISAs.

Within this study, the significance of utilizing a tissue-mimetic environment for cell culture is highlighted and the role of maintaining a more regenerative MSC-like population for development of regenerative therapies is evaluated. More specifically, the ability for the different 3D ASC-CM groups to display varying functional capabilities, whereas 2D ASC-CM groups displayed minimal differences between the different coating groups supports the hypothesis that ASC are able to adapt to environmental stimuli more readily within the tissue-mimetic system. The authors demonstrate the tailorable role that dimensionality, mechanics, and substrate binding have on influencing ASC secretory activity and the subsequent functional changes seen within the ASC secretome. These parameters are only a small part of a larger understanding, but demonstrate that in vitro systems looking to control MSC-like populations and their secretome must consider a multitude of parameters to truly understand and control their activity. A variety of other parameters to consider include the incorporation of environmental (e.g., hypoxia), chemical (e.g., growth factors/cytokines), mechanical (e.g., dynamic compression), or biological stimuli (e.g., co-culture), as well as the addition of specific drugs to better understand the role of the secretome in drug response. Thus, this study offers insight into the future development and potential of tailorable acellular byproducts that can be prefabricated for a multitude of soft tissue wound healing applications, as well as providing the opportunity to more effectively evaluate native cell responses to a variety of stimuli.

### 4. Conclusion

Ultimately, previous studies have demonstrated that ASCs can be primed with exposure to different compounds and environmental conditions to enhance specific functions in vivo (e.g., hypoxia priming to enhance angiogenic activity). However, this study offers insight into the comparative effects of matrix-derived compounds on the ASC secretome functionality in regard to epidermal regeneration of idKCs. The potential benefits of tailored ASC biologics on the epidermal regeneration capacity has yet to be demonstrated. Within this study, the role of ASC-secretome on modulating key epidermal regeneration signaling pathways in idKCs is revealed, including the ability to alter keratin expression, promote suprabasal differentiation, and improve barrier formation. More specifically, the role of ASC-EVs within the ASC secretome was evaluated and shown to be a potential key driver of idKC functional responses, suggesting a dynamic shift in signaling towards favoring EV-like compounds when KCs assume a diabetic-like phenotype. Moreover, this study offers insight into the critical role of tissue-mimetic culture on achieving robust MSC-like populations that retain a more dynamic secretory phenotype and are able to adapt more readily to changes in environmental conditions. Whereas 2D culture resulted in significantly less changes when exposed to different coating substrates. Thus, by introducing predetermined exogenous stimuli within a tissue-mimetic system, the secretory product composition can be tailored and fine-tuned, providing an opportunity to generate an array of functional diverse biologics for different applications.

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A method of characterizing a therapeutically desirable constituent or constituent profile of a secretome composition secreted from a cell type of interest, comprising:
(i) seeding a first tissue mimetic three-dimensional (3D) hydrogel block (TMHB) comprising a macrostructure defined by a continuous polymeric matrix material and a network of microporous channels and/or chambers extending throughout the continuous polymeric matrix material with a first cell type of interest;
(ii) simulating in vitro cell growth and propagation of the first cell type of interest by feeding the first cell type of interest with a first culture media and allowing the first cell type of interest to propagate throughout the first TMHB and to secrete the secretome composition;
(iv) collecting a secretome composition;
(v) subjecting the secretome composition to a stratification operation based on molecular weight cut-off values providing a plurality of different strata each characterized by a different range of constituent-molecular weights;
(vi) characterizing and/or identifying one or more individual constituents present with each strata of interest; and
(v) testing each strata of interest for relative therapeutic benefit either by in vivo analysis or ex vivo analysis and identifying the strata of interest that provides a highest therapeutic benefit as the therapeutically constituent profile.

2. The method of claim 1, further comprising characterizing at least a majority (e.g., each) of the individual constituents present in the strata of interest that provides a highest therapeutic benefit.

3. The method of claim 2, further comprising a step of identifying at least one high occurrence constituent from the one or more individual constituents present in the strata of interest that provides a highest therapeutic benefit that is either not found or found in a lesser amount in each of the strata of interest to identify the at least one high occurrence constituent as the therapeutically desirable constituent.

4. A method of tailoring a constituent profile of a natural secretome composition secreted from a cell type of interest, comprising:
(i) seeding a first tissue mimetic three-dimensional (3D) hydrogel block (TMHB) comprising a macrostructure defined by a continuous polymeric matrix material and a network of microporous channels and/or chambers extending throughout the continuous polymeric matrix material with a first cell type of interest;
(ii) feeding first cell type of interest with a first culture media;
(iii) introducing at least one external stimuli configured to modify the natural secretome composition secreted from a cell type of interest to more closely track a target secretome composition having a target constituent or target constituent profile; wherein the at least one external stimuli comprises one or more controllable parameters, such as addition of a coating on an interface between the continuous polymeric matrix material and the network of microporous channels and/or chambers, adjusting an oxygen concentration, a pH, a temperature, and fluid dynamics through the first TMHB;
(iv) allowing the first cell type of interest to propagate throughout the first TMHB and to secrete the secretome composition;
(iv) collecting a modified secretome composition that is different than the natural secretome composition due to the external stimuli.
